(19) United States
(12) Patent Application Publication
(10) Pub. No.: US 2006/0099623 A1
(43) Pub. Date: May 11, 2006

(54) Polynucleotides and polypeptides isolated from lactobacillus and methods for their use

(76) Inventors: Matthew Glenn, Palmerston North (NZ); Mark W. Lubbers, Palmerston North (NZ); James Dekker, Palmerston North (NZ)

Correspondence Address: SPECKMAN LAW GROUP PLLC, 1201 THIRD AVENUE, SUITE 330, SEATTLE, WA, 98101, US

(21) Appl. No.: 11/249,111

(22) Filed: Oct. 11, 2005

Related U.S. Application Data
(63) Continuation-in-part of application No. 10/288,930, filed on Nov. 5, 2002.
Application No. 10/288,930 is a continuation of application No. 09/724,623, filed on Nov. 28, 2000, now Pat. No. 6,476,209.

(51) Int. Cl. C12Q 1/68; C07H 21/04

(52) U.S. Cl. 435/6; 536/23.1

(57) ABSTRACT

Novel polynucleotides isolated from Lactobacillus rhamnosus, as well as probes and primers, genetic constructs comprising the polynucleotides, biological materials, including plants, microorganisms and multicellular organisms incorporating the polynucleotides, polypeptides expressed by the polynucleotides, and methods for using the polynucleotides and polypeptides are disclosed.

### CROSS-REFERENCE TO RELATED APPLICATIONS

This application is a continuation of U.S. patent application Ser. No. 10/288,930, filed Nov. 5, 2002, which is a continuation of U.S. patent application Ser. No. 09/724,623, filed Nov. 28, 2000, now U.S. Pat. No. 6,476,209, which claims to priority under 35 U.S.C. §119(e) to U.S. Provisional Patent Application 60/148,801, filed Dec. 2, 1999.

### REFERENCE TO SEQUENCE LISTING SUBMITTED ON COMPACT DISC

This application incorporates by reference in its entirety the Sequence Listing contained in the accompanying two compact discs, one of which is a duplicate copy. Each CD contains a single file, named “1048u1c2 SEQLIST.txt,” the size of which is 252 KB, and which was created on Oct. 11, 2005, in IBM-PC MS-Windows 2000 format pursuant to 37 CFR §1.52 (e).

### TECHNICAL FIELD OF THE INVENTION

This invention relates to polynucleotides isolated from lactic acid bacteria, as well as to probes and primers specific to the polynucleotides; DNA constructs comprising the polynucleotides; biological materials, including plants, microorganisms and multicellular organisms, incorporating the polynucleotides; polypeptides expressed by the polynucleotides; and methods for using the polynucleotides and polypeptides.

### BACKGROUND OF THE INVENTION

The present invention relates to polynucleotides isolated from a specific strain of lactic acid bacteria, namely Lactobacillus rhamnosus HN001 (L. rhamnosus HN001). Lactic acid bacteria, and their enzymes, are the major determinants of flavor and fermentation characteristics in fermented dairy products, such as cheese and yogurt. Flavors are produced through the action of bacteria and their enzymes on proteins, carbohydrates and lipids.

Lactobacillus rhamnosus strain HN001 are heterofermentative bacteria that are Gram positive, non-motile, non-spore forming, catalase negative, facultative anaerobic rods exhibiting an optimal growth temperature of 37±1° C. and an optimum pH of 6.0-6.5. Experimental studies demonstrated that dietary supplementation with Lactobacillus rhamnosus strain HN001 induced a sustained enhancement in several aspects of both natural and acquired immunity (See PCT International Publication No. WO 99/10476). In addition, L. rhamnosus HN001, and certain other Gram-positive bacteria can specifically and directly modulate human and animal health (See, for example, Tannock et al., Applied Environ. Microbiol. 66:2578-2588, 2000; Gill et al., Brit. J. Nutrition 83:167-176; Quan Shu et al., Food and Chem. Toxicol. 38:153-161, 2000; Quan Shu et al., Intl. J. Food Microbiol. 56:87-96, 2000; Quan Shu et al., Intl. Dairy J. 9:831-836, 1999; Prasad et al., Intl. Dairy J. 8:993-1002, 1998; Sanders and Huis in't Veld, Antonie van Leeuwenhoek 76:293-315, 1999; Salminen et al., 1998. In: Lactic Acid Bacteria, Salminen S and von Wright A (eds)., Marcel Dekker Inc, New York, Basel, Hong Kong, pp. 211-253; Delcour et al., Antonie van Leeuwenhoek 76:159-184, 1999; Blum et al., Antonie van Leeuwenhoek 76:199-205, 1999; Yasui et al., Antonie van Leeuwenhoek 76:383-389, 1999; Hirayama and Rafter, Antonie van Leeuwenhoek 76:391-394, 1999; Ouwehand, 1998. In: Lactic Acid Bacteria, Salminen S and von Wright A (eds)., Marcel Dekker Inc, New York, Basel, Hong Kong, pp. 139-159; Isolauri et al., S 1998. In: Lactic Acid Bacteria, Salminen S and von Wright A (eds)., Marcel Dekker Inc, New York, Basel, Hong Kong, pp. 255-268; Lichtenstein and Goldin, 1998. In: Lactic Acid Bacteria, Salminen S and von Wright A (eds)., Marcel Dekker Inc, New York, Basel, Hong Kong, pp. 269-277; El-Nezami and Ahokas, 1998. In: Lactic Acid Bacteria, Salminen S and von Wright A (eds)., Marcel Dekker Inc, New York, Basel, Hong Kong, pp. 629-367; Nousianen et al., 1998. In: Lactic Acid Bacteria, Salminen S and von Wright A (eds)., Marcel Dekker Inc, New York, Basel, Hong Kong, pp. 437-473; Meisel and Bockelmann, Antonie van Leeuwenhoek 76:207-215, 1999; Christensen et al., Antonie van Leeuwenhoek 76:217-246, 1999; Dunne et al., Antonie van Leeuwenhoek 76:279-292, 1999).

Beneficial health effects attributed to these bacteria include the following:
Increased resistance to enteric pathogens and anti-infection activity, including treatment of rotavirus infection and infantile diarrhea—due to increases in antibody production caused by an adjuvant effect, increased resistance to pathogen colonization; alteration of intestinal conditions, such as pH; and the presence of specific antibacterial substances, such as bacteriocins and organic acids.
Aid in lactose digestion—due to lactose degradation by bacterial lactase enzymes (such as beta-galactosidase) that act in the small intestine.
Anti-cancer (in particular anti-colon cancer) and anti-mutagenesis activities—due to anti-mutagenic activity; alteration of procancerous enzymatic activity of colonic microbes; reduction of the carcinogenic enzymes azoreductase, beta-glucuronidase and nitroreductase in the gut and/or faeces; stimulation of immune function; positive influence on bile salt concentration, and antioxidant effects.
Liver cancer reduction—due to aflatoxin detoxification and inhibition of mould growth.
Reduction of small bowel bacterial overgrowth—due to antibacterial activity; and decrease in toxic metabolite production from overgrowth flora.
Immune system modulation and treatment of autoimmune disorders and allergies—due to enhancement of non-specific and antigen-specific defence against infection and tumors; enhanced mucosal immunity; adjuvant effect in antigen-specific immune responses; and regulation of Th1/Th2 cells and production of cytokines.
Treatment of allergic responses to foods—due to prevention of antigen translocation into blood stream and modulation of allergenic factors in food.
Reduction of blood lipids and prevention of heart disease—due to assimilation of cholesterol by bacteria; hydrolysis of bile salts; and antioxidative effects.
Antihypertensive effect—bacterial protease or peptidase action on milk peptides produces antihypertensive peptides. Cell wall components act as ACE inhibitors
Prevention and treatment of urogenital infections—due to adhesion to urinary and vaginal tract cells resulting in competitive exclusion; and production of antibacterial substances (acids, hydrogen peroxide and biosurfactants).
Treatment of inflammatory bowel disorder and irritable bowel syndrome—due to immuno-modulation; increased resistance to pathogen colonization; alteration of intestinal conditions such as pH; production of specific antibacterial substances such as bacteriocins, organic acids and hydrogen peroxide and biosurfactants; and competitive exclusion.
Modulation of infective endocarditis—due to fibronectin receptor-mediated platelet aggregation associated with Lactobacillus sepsis.
Prevention and treatment of Helicobacter pylori infection—due to competitive colonization and antibacterial effect.
Prevention and treatment of hepatic encephalopathy—due to inhibition and/or exclusion of urease-producing gut flora.
Improved protein and carbohydrate utilization and conversion—due to production of beneficial products by bacterial action on proteins and carbohydrates.

Other beneficial health effects associated with L. rhamnosus include: improved nutrition; regulation of colonocyte proliferation and differentiation; improved lignan and isoflavone metabolism; reduced mucosal permeability; detoxification of carcinogens and other harmful compounds; relief of constipation and diarrhea; and vitamin synthesis, in particular folate.

Peptidases are enzymes that break the peptide bonds linking the amino group of one amino acid with the carboxy group (acid group) of an adjacent amino acid in a peptide chain. The bonds are broken in a hydrolytic reaction. There is a large family of peptidase enzymes that are defined by their specificity for the particular peptide bonds that they cleave (Barrett A J, Rawlings N D and Woessner J F (Eds.) 1998. Handbook of proteolytic enzymes. Academic Press, London, UK). The two main families are exopeptidases and endopeptidases.

Exopeptidases cleave amino acids from the N— or C-terminus of a peptide chain, releasing free amino acids or short (di- and tri-) peptides. Different types of exopeptidases include:
Aminopeptidases—release a free amino acid from the N-terminus of a peptide chain;
dipeptidyl-peptidases (also known as dipeptidyl-aminopeptidases)—release a dipeptide from the N-terminus of a peptide chain;
tripeptidyl-peptidases (also known as tripeptidyl-aminopeptidases)—release a tripeptide from the N-terminus of a peptide chain);
carboxypeptidases—release a free amino acid from the C-terminus of a peptide chain;
peptidyl-dipeptidase—release a dipeptide from the C-terminus of a peptide chain;
dipeptidases—release two free amino acids from a dipeptide; and
tripeptidases—release a free amino acid and a dipeptide from a tripeptide.

Endopeptidases hydrolyze peptide bonds internally within a peptide and are classified on the basis of their mode of catalysis:
serine-endopeptidases—depend on serine (or threonine) as the nucleophile in the catalytic reaction;
cysteine-endopeptidases—depend on the sulphydryl group of cysteine as the nucleophile in the catalytic reaction;
aspartic-endopeptidases—contain aspartate residues that act as ligands for an activated water molecule which acts as the nucleophile in the catalytic reaction; and
metallo-endopeptidases—contain one or more divalent metal ions that activate the water molecule that acts as the nucleophile in the catalytic reaction.

Peptidases are important enzymes in the process of cheese ripening and the development of cheese flavor. The hydrolysis of milk caseins in cheese results in textural changes and the development of cheese flavors. The raft of proteolytic enzymes that cause this hydrolysis come from the lactic acid bacteria that are bound up in the cheese—either starter cultures that grow up during the manufacture of the cheese, or adventitious and adjunct non-starter lactic acid bacteria that grow in the cheese as it ripens (Law Haandrikman, Int. Dairy J. 7:1-11, 1997).

Many other enzymes can also influence dairy product flavor, and functional and textural characteristics, as well as influencing the fermentation characteristics of the bacteria, such as speed of growth, acid production and survival (Urbach, Int. Dairy J. 5:877-890, 1995; Johnson and Somkuti, Biotech. Appl. Biochem. 13:196-204, 1991; El Soda and Pandian, J. Dairy Sci. 74:2317-2362, 1991; Fox et al. In Cheese: chemistry, physics and microbiology. Volume 1, General aspects, 2^{nd} edition, P Fox (ed) Chapman and Hall, London; Christensen et al., Antonie van Leeuwenhoek 76:217-246, 1999; Stingle et al., J. Bacteriol. 20:6624-6360, 1999; Stingle et al., Mol. Microbiol. 32:1287-1295, 1999; Lemoine et al., Appl. Environ. Microbiol. 63:1512-6218, 1997).

Enzymes influencing specific characteristics and/or functions include the following:
Lysis of cells. These enzymes are mostly cell wall hydrolases, including amidases; muramidases; lysozymes, including N-acetyl muramidase; muramidase; N-acetyl-glucosaminidase; and N-acetylmuramoyl-L-alanine amidase. DEAD-box helicase proteins also influence autolysis.
Carbohydrate utilization. Lactose, citrate and diacetyl metabolism, and alcohol metabolism are particularly important. The enzymes involved include beta-galactosidase, lactate dehydrogenase, citrate lyase, citrate permease, 2,3 butanediol dehydrogenase (acetoin reductase), acetolactate decarboxylase, acetolactate synthase, pyruvate decarboxylase, pyruvate formate lyase, diacetyl synthase, diacetyl reductase, alcohol decarboxylase, lactate dehydrogenase, pyruvate dehydrogenase, and aldehyde dehydrogenase.
Lipid degradation, modification or synthesis. Enzymes involved include lipases, esterases, phospholipases, serine hydrolases, desaturases, and linoleate isomerase.
Polysaccharide synthesis. Polysaccharides are important not only for potential immune enhancement and adhesion activity but also for the texture of fermented dairy products. The enzymes involved are a series of glucosyl transferases, including beta-(1-3)-glucosyl transferase, alpha-N acetylgalactosaminyl transferase, phosphogalactosyl transferase, alpha-glycosyl transferase, UDP-N-acetylglucosamine C4 epimerase and UDP-N-acetylglucosamine transferase.
Amino acid degradation. Enzymes involved include glutamate dehydrogenase, aminotransferases, amino acid decarboxylases, and enzymes involved in sulfur amino acid degradation including cystothione beta-lyase.

Sequencing of the genomes, or portions of the genomes, of numerous organisms, including humans, animals, microorganisms and various plant varieties, has been and is being carried out on a large scale. Polynucleotides identified using sequencing techniques may be partial or full-length genes, and may contain open reading frames, or portions of open reading frames, that encode polypeptides. Putative polypeptides may be identified based on polynucleotide sequences and further characterized. The sequencing data relating to polynucleotides thus represents valuable and useful information.

Polynucleotides and polypeptides may be analyzed for varying degrees of novelty by comparing identified sequences to sequences published in various public domain databases, such as EMBL. Newly identified polynucleotides and corresponding putative polypeptides may also be compared to polynucleotides and polypeptides contained in public domain information to ascertain homology to known polynucleotides and polypeptides. In this way, the degree of similarity, identity or homology of polynucleotides and polypeptides having an unknown function may be determined relative to polynucleotides and polypeptides having known functions.

Information relating to the sequences of isolated polynucleotides may be used in a variety of ways. Specified polynucleotides having a particular sequence may be isolated, or synthesized, for use in in vivo or in vitro experimentation as probes or primers. Alternatively, collections of sequences of isolated polynucleotides may be stored using magnetic or optical storage medium and analyzed or manipulated using computer hardware and software, as well as other types of tools.

### SUMMARY OF THE INVENTION

The present invention provides isolated polynucleotides comprising a sequence selected from the group consisting of: (a) sequences identified in the attached Sequence Listing as SEQ ID NO: 1-62; (b) variants of those sequences; (c) extended sequences comprising the sequences set out in SEQ ID NO: 1-62 and their variants; and (d) sequences comprising at least a specified number of contiguous residues of a sequence of SEQ ID NO: 1-62 (x-mers). Oligonucleotide probes and primers corresponding to the sequences set out in SEQ ID NO: 1-62, and their variants are also provided. All of these polynucleotides and oligonucleotide probes and primers are collectively referred to herein, as “polynucleotides of the present invention.”

The polynucleotide sequences identified as SEQ ID NO: 1-62 were derived from a microbial source, namely from fragmented genomic DNA of Lactobacillus rhamnosus, strain HN001, described in PCT Patent Publication WO 99/10476. As discussed above, Lactobacillus rhamnosus strain HN001 are heterofermentative bacteria that are Gram positive, non-motile, non-spore forming, catalase negative, facultative anaerobic rods exhibiting an optimal growth temperature of 37±1° C. and an optimum pH of 6.0-6.5. Experimental studies have demonstrated that dietary supplementation with Lactobacillus rhamnosus strain HN001 induces a sustained enhancement in several aspects of both natural and acquired immunity. A biologically pure culture of Lactobacillus rhamnosus strain HN001 was deposited at the Australian Government Analytical Laboratories (AGAL), The New South Wales Regional Laboratory, 1 Suakin Street, Pymble, NSW 2073, Australia, as Deposit No. NM97/09514, dated 18 Aug. 1997.

Certain of the polynucleotide sequences disclosed herein may be “partial” sequences in that they do not represent a full-length gene encoding a full-length polypeptide. Such partial sequences may be extended by analyzing and sequencing various DNA libraries using primers and/or probes and well-known hybridization and/or PCR techniques. The partial sequences disclosed herein may thus be extended until an open reading frame encoding a polypeptide, a full-length polynucleotide and/or gene capable of expressing a polypeptide, or another useful portion of the genome is identified. Such extended sequences, including full-length polynucleotides and genes, are described as “corresponding to” a sequence identified as one of the sequences of SEQ ID NO: 1-62 or a variant thereof, or a portion of one of the sequences of SEQ ID NO: 1-62 or a variant thereof, when the extended polynucleotide comprises an identified sequence or its variant, or an identified contiguous portion (x-mer) of one of the sequences of SEQ ID NO: 1-62 or a variant thereof.

The polynucleotides identified as SEQ ID NO: 1-62 were isolated from Lactobacillus rhamnosus genomic DNA clones and represent sequences that are present in the cells from which the DNA was prepared. The sequence information may be used to identify and isolate, or synthesize, DNA molecules such as promoters, DNA-binding elements, open reading frames or full-length genes, that can then be used as expressible or otherwise functional DNA in transgenic organisms. Similarly, RNA sequences, reverse sequences, complementary sequences, antisense sequences and the like corresponding to the polynucleotides of the present invention may be routinely ascertained and obtained using the polynucleotides identified as SEQ ID NO: 1-62.

The present invention further provides isolated polypeptides encoded, or partially encoded, by the polynucleotides disclosed herein. In certain specific embodiments, the polypeptides of the present invention comprise a sequence selected from the group consisting of sequences identified as SEQ ID NO: 63-124, and variants thereof. Polypeptides encoded by the polynucleotides of the present invention may be expressed and used in various assays to determine their biological activity. Such polypeptides may be used to raise antibodies, to isolate corresponding interacting proteins or other compounds, and to quantitatively determine levels of interacting proteins or other compounds.

Genetic constructs comprising the inventive polynucleotides are also provided, together with transgenic host cells comprising such constructs and transgenic organisms, such as microbes, comprising such cells. Preferably the transgenic organisms are non-human.

The present invention also contemplates methods for modulating the polynucleotide and/or polypeptide content and composition of an organism, such methods involving stably incorporating into the genome of the organism a genetic construct comprising a polynucleotide of the present invention. In one embodiment, the target organism is a microbe, preferably a microbe used in fermentation, more preferably a microbe of the genus Lactobacillus, and most preferably Lactobacillus rhamnosus, or other closely microbial related species used in the dairy industry. In a related aspect, methods for producing a microbe having an altered genotype and/or phenotype is provided, such methods comprising transforming a microbial cell with a genetic construct of the present invention to provide a transgenic cell, and cultivating the transgenic cell under conditions conducive to growth and multiplication. Organisms having an altered genotype or phenotype as a result of modulation of the level or content of a polynucleotide or polypeptide of the present invention compared to a wild-type organism, as well as components and progeny of such organisms, are contemplated by and encompassed within the present invention.

The isolated polynucleotides of the present invention may be usefully employed for the detection of lactic acid bacteria, preferably L. rhamnosus, in a sample material, using techniques well known in the art, such as polymerase chain reaction (PCR) and DNA hybridization, as detailed below.

The inventive polynucleotides and polypeptides may also be employed in methods for the selection and production of more effective probiotic bacteria; as “bioactive” (health-promoting) ingredients and health supplements, for immune function enhancement; for reduction of blood lipids such as cholesterol; for production of bioactive material from genetically modified bacteria; as adjuvants; for wound healing; in vaccine development, particularly mucosal vaccines; as animal probiotics for improved animal health and productivity; in selection and production of genetically modified rumen microorganisms for improved animal nutrition and productivity, better flavor and improved milk composition; in methods for the selection and production of better natural food bacteria for improved flavor, faster flavor development, better fermentation characteristics, vitamin synthesis and improved textural characteristics; for the production of improved food bacteria through genetic modification; and for the identification of novel enzymes for the production of, for example, flavors or aroma concentrates.

The isolated polynucleotides of the present invention also have utility in genome mapping, in physical mapping, and in positional cloning of genes of more or less related microbes.

Additionally, the polynucleotide sequences identified as SEQ ID NO: 1-62, and their variants, may be used to design oligonucleotide probes and primers. Oligonucleotide probes and primers have sequences that are substantially complementary to the polynucleotide of interest over a certain portion of the polynucleotide. Oligonucleotide probes designed using the polynucleotides of the present invention may be used to detect the presence and examine the expression patterns of genes in any organism having sufficiently similar DNA and RNA sequences in their cells, using techniques that are well known in the art, such as slot blot DNA hybridization techniques. Oligonucleotide primers designed using the polynucleotides of the present invention may be used for PCR amplifications. Oligonucleotide probes and primers designed using the polynucleotides of the present invention may also be used in connection with various microarray technologies, including the microarray technology of Affymetrix (Santa Clara, Calif.).

The polynucleotides of the present invention may also be used to tag or identify an organism or derived material or product therefrom. Such tagging may be accomplished, for example, by stably introducing a non-disruptive non-functional heterologous polynucleotide identifier into an organism, the polynucleotide comprising at least a portion of a polynucleotide of the present invention.

The polynucleotides of the present invention may additionally be used as promoters, gene regulators, origins of DNA replication, secretion signals, cell wall or membrane anchors for genetic tools (such as expression or integration vectors).

All references cited herein, including patent references and non-patent publications, are hereby incorporated by reference in their entireties.

### DETAILED DESCRIPTION

The polynucleotides disclosed herein were isolated by high throughput sequencing of DNA libraries from the lactic acid bacteria Lactobacillus rhamnosus as described in Example 1 below. Cell wall, cell surface and secreted components of lactic acid bacteria are known to mediate immune modulation, cell adhesion and antibacterial activities, resulting in many beneficial effects including: resistance to enteric pathogens; modulation of cancer, including colon cancer; anti-mutagenesis effects; reduction of small bowel bacterial overgrowth; modulation of auto-immune disorders; reduction in allergic disorders; modulation of urogenital infections, inflammatory bowel disorder, irritable bowel syndrome, Helicobacter pylori infection and hepatic encephalopathy; reduction of infection with pathogens; regulation of colonocyte proliferation and differentiation; reduction of mucosal permeability; and relief of constipation and diarrhea. These cell components include, but are not limited to, peptidoglycans, teichoic acids, lipoteichoic acids, polysaccharides, adhesion proteins, secreted proteins, surface layer or S-layer proteins, collagen binding proteins and other cell surface proteins, and antibacterial substances such as bacteriocins and organic acids produced by these bacteria. Polynucleotides involved in the synthesis of these proteins and in the synthesis, modification, regulation, transport, synthesis and/or accumulation of precursor molecules for these proteins can be used to modulate the immune, antibacterial, cell adhesion and competitive exclusion effects of the bacteria or of components that might be produced by these bacteria.

In order to function effectively as probiotic bacteria, L. rhamnosus HN001 must survive environmental stress conditions in the gastrointestinal tract, as well as in commercial and industrial processes. Modification of particular polynucleotides or regulatory processes has been shown to be effective against a number of stresses including oxidative stress, pH, osmotic stress, dehydration, carbon starvation, phosphate starvation, nitrogen starvation, amino acid starvation, heat or cold shock, and mutagenic stress. Polynucleotides involved in stress resistance often confer multistress resistance, i.e., when exposed to one stress, surviving cells are resistant to several non-related stresses. Bacterial genes and/or processes shown to be involved in multistress resistance include:
Intracellular phosphate pools—inorganic phosphate starvation leads to the induction of pho regulon genes, and is linked to the bacterial stringent response. Gene knockouts involving phosphate receptor genes appear to lead to multistress resistance.
Intracellular guanosine pools—purine biosynthesis and scavenger pathways involve the production of phosphate-guanosine compounds that act as signal molecules in the bacterial stringent response. Gene knockouts involving purine scavenger pathway genes appear to confer multistress resistance.
Osmoregulatory molecules—small choline-based molecules, such as glycine-betaine, and sugars, such as trehalose, are protective against osmotic shock and are rapidly imported and/or synthesized in response to increasing osmolarity.
Acid resistance—lactobacilli naturally acidify their environment through the excretion of lactic acid, mainly through the cit operon genes responsible for citrate uptake and utilization.
Stress response genes—a number of genes appear to be induced or repressed by heat shock, cold shock, and increasing salt through the action of specific promoters.

The isolated polynucleotides of the present invention, and genetic constructs comprising such polynucleotides”, may be employed to produce bacteria having desired phenotypes, including increased resistance to stress and improved fermentation properties.

Many enzymes are known to influence dairy product flavor, functional and textural characteristics as well as general fermentation characteristics such as speed of growth, acid production and survival. These enzymes include those involved in the metabolism of lipids, polysaccharides, amino acids and carbohydrates, as well as those involved in the lysis of the bacterial cells.

The isolated polynucleotides and polypeptides of the present invention have demonstrated similarity to polynucleotides and/or polypeptides of known function. The identity and functions of the inventive polynucleotides based on such similarities are shown below in Table 1.

TABLE 1

| SEQ ID NO: | SEQ ID NO: | |
|---|---|---|
| Polynucleotide | Polypeptide | Gene function or protein class |
| 1 | 63 | Transmembrane protein that participates in the adhesion of |
| | | bacteria to gut cells, part of an operon containing the mapA |
| | | gene encoding a mucin binding protein. This gene may be used |
| | | to identify or manipulate interactions with gut cells. |
| 2 | 64 | Common 28 kDa antigen and major cell adherence molecule of |
| | | Campylobacter jejuni and Campylobacter coli. Significant |
| | | similarity to amino acid transport proteins in Gram-negative |
| | | bacteria. This gene may be used to identify or manipulate both |
| | | interactions with gut cells and amino acid metabolism. |
| 3 | 65 | Histidinol-phosphate aminotransferase, may also have tyrosine |
| | | and phenylalanine aminotransferase activity. Involved in |
| | | amino acid metabolism. May be used to identify or manipulate |
| | | metabolism and influence growth and the production of flavor |
| | | compounds. |
| 4 | 66 | Aspartate transaminase (EC 2.6.1.1). Converts L-aspartate and |
| | | 2-oxoglutarate to oxaloacetate and L-glutamate, but may also |
| | | be involved in aromatic amino acid, alanine, cysteine, proline, |
| | | and asparagine pathways. Its role amino acid metabolism |
| | | suggests impact in production of flavor compounds, and may |
| | | also be involved in carbon fixation. May be used to identify or |
| | | manipulate metabolism and influence growth and the |
| | | production of flavor compounds. |
| 5 | 67 | Aromatic amino acid transferase. It is used to identify or |
| | | manipulate metabolism and influence growth and the |
| | | production of flavor compounds. |
| 6 | 68 | Tyrosine aminotransferase (EC 2.6.1.5) (L-tyrosine: 2- |
| | | oxoglutarate aminotransferase). Transfers nitrogenous groups |
| | | as part of the aromatic amino acid pathway. Involved in |
| | | synthesis of flavor compounds and amino acid metabolism. It |
| | | is used to identify or manipulate metabolism and influence |
| | | growth and the production of flavor compounds. |
| 7 | 69 | Aminotransferase B. Probable aminotransferase belonging to |
| | | class-II pyridoxal-phosphate-dependent aminotransferase |
| | | family. It is used to identify or manipulate metabolism and |
| | | influence growth and the production of flavor compounds. |
| 8 | 70 | Cysteine desulfurase, a class-V aminotransferase that supplies |
| | | inorganic sulfide for Fe-S clusters. Involved in cysteine |
| | | metabolism and generation of flavor compounds. It is used to |
| | | identify or manipulate metabolism and influence growth and |
| | | the production of flavor compounds. |
| 9 | 71 | Lipase, breakdown of triglycerides. It is used to identify or |
| | | manipulate metabolism and influence growth and the |
| | | production of flavor compounds. |
| 10 | 72 | O-acetylserine sulfhydrylase involved in cysteine synthesis. |
| | | Converts O-acetyl-L-serine and H2S to L-cysteine and acetate. |
| | | Involved in synthesis of flavor and aroma compounds. It is |
| | | used to identify or manipulate metabolism and influence |
| | | growth and the production of flavor compounds. |
| 11 | 73 | Surface protein thought to be involved in a number of functions |
| | | including as a collagen and/or mucin binding protein in cellular |
| | | adhesion and as a cysteine transporter, part of the ABC |
| | | superfamily, which affects amino acid metabolism and flavor |
| | | compound synthesis. It is used to identify or manipulate |
| | | metabolism, growth, the production of flavor compounds, and |
| | | interactions with gut cells. |
| 12 | 74 | Group B streptococcal oligopeptidase, degrades a variety of |
| | | bioactive peptides. Involved in protein breakdown and |
| | | metabolism, and may modify flavor compounds as well modify |
| | | health through the stability or production of bioactive peptides. |
| 13 | 75 | Pz-peptidase, a metalloproteinase and part of the thimet |
| | | oligopeptidase family: Hydrolyses the Pz-peptide, 4- |
| | | phenylazobenzyloxycarbonyl-Pro-Leu-Gly-Pro-Arg. It impacts |
| | | on flavor compounds as well modify health through the |
| | | stability or production of bioactive peptides. |
| 14 | 76 | Adenosine triphosphatase clpC. ATP-dependent Clp proteinase |
| | | regulatory protein, a pleiotropic regulator controlling growth at |
| | | high temperatures. Involved in stress response. It is used to |
| | | identify or modify the survival or virulence of organisms. |
| 15 | 77 | Streptococcal C5a peptidase. Specifically cleaves human |
| | | serum chemotaxin C5a near its C-terminus, destroying its |
| | | ability to serve as a chemoattractant. It mediates interactions |
| | | with host immune system and is used to identify or modify |
| | | interactions with immune systems. |
| 16 | 78 | Dipeptidase from Lactococcus lactis. Hydrolyzes a broad |
| | | range of dipeptides but no tri, tetra, or larger oligopeptides. It |
| | | is used to identify or modify protein metabolism and flavor |
| | | compound synthesis. |
| 17 | 79 | Acylamino-acid-releasing enzyme (acyl-peptidehydrolase or |
| | | acylaminoacyl-peptidase) EC 3.4.19.1. Catalyzes removal N |
| | | alpha-acetylated amino acid residues from N alpha-acetylated |
| | | peptides. It is used to identify or modify metabolism or flavor |
| | | or aroma compound production. |
| 18 | 80 | Heat shock protease regulatory subunit, the ATPase subunit of |
| | | an intracellular ATP-dependent protease. It is used to identify |
| | | or modify survival or virulence. |
| 19 | 81 | O-sialoglycoprotein endopeptidase (EC 3.4.24.57). Hydrolyses |
| | | O-sialoglycoproteins, but does not cleave unglycosylated |
| | | proteins, desialylated glycoproteins or N-glycosylated |
| | | glycoproteins. Sialogylcoproteins can act as receptors for |
| | | adhesion to gut cells. It is used to identify or modify |
| | | interactions with gut cells, protein metabolism, stability or |
| | | production of bioactive peptides. |
| 20 | 82 | Carboxylesterase, converts a carboxylic ester to an alcohol and |
| | | a carboxylic acid anion. Esters and alcohols can be potent |
| | | flavor and aroma compounds. It is used to identify or modify |
| | | metabolism or flavor or aroma compound production. |
| 21 | 83 | Glycerophosphodiester phosphodiesterase. Converts |
| | | glycerophosphodiesters to an alcohol and glycerol 3-phosphate. |
| | | Alcohols are potentially important flavor compounds. It is used |
| | | to identify or modify metabolism or flavor or aroma compound |
| | | production. |
| 22 | 84 | Bifunctional alcohol dehydrogenase and acetaldehyde |
| | | dehydrogenase. Ferments glucose to ethanol under anaerobic |
| | | conditions. It is used to identify or modify metabolism or |
| | | flavor or aroma compound production. |
| 23 | 85 | Short-chain alcohol dehydrogenase. It is used to identify or |
| | | modify metabolism or flavor or aroma compound production. |
| 24 | 86 | Aryl-alcohol dehydrogenase. Converts an aromatic alcohol to |
| | | an aromatic aldehyde. It is used to identify or modify |
| | | metabolism or flavor or aroma compound production. |
| 25 | 87 | Branched chain amino acid transport system II carrier protein, |
| | | involved in amino acid metabolism. Amino acid metabolism is |
| | | important in flavor compound production. It is used to identify |
| | | or modify metabolism or flavor compound production. |
| 26 | 88 | Human bile salt export pump. Bile tolerance is an important |
| | | property of probiotic bacteria. Bile salt removal can reduce |
| | | cholesterol. May be used to identify or modify bile tolerance or |
| | | cholesterol reduction. |
| 27 | 89 | Bifunctional HPr Kinase/P-Ser-HPr phosphatase from |
| | | Lactobacillus casei. Controls catabolite repression and |
| | | involved in phosphate regulation. Phosphate regulation is |
| | | important in cell survival and stress tolerance. It is used to |
| | | identify or modify gene regulation and on stress tolerance. |
| 28 | 90 | Suppressor of dominant negative ftsH mutations affecting |
| | | extracellular protein transport in E. coli. It is used to identify or |
| | | modify protein transport. |
| 29 | 91 | Malolactic enzyme. Converts between malate and lactate. |
| | | Central to carbohydrate metabolism, also involved in acid |
| | | tolerance. It is used to identify or modify metabolism or flavor |
| | | compound production or cell survival. |
| 30 | 92 | Magnesium transporter, also has affinity for cobalt. Metal ion |
| | | transport is involved in bacterial survival as well as other |
| | | aspects of metabolism. It is used to identify or modify |
| | | metabolism or cell survival. |
| 31 | 93 | Pyruvate dehydrogenase E1 (lipoamide) alpha subunit (EC |
| | | 1.2.4.1). Glycolytic enzyme, also involved in branched-chain |
| | | amino acid synthesis. It is used to identify or modify |
| | | metabolism or flavor or aroma compound production. |
| 32 | 94 | Adhesin involved in diffuse adherence of diarrhoeagenic E. coli. |
| | | May be used to identify or modifyinteractions with gut |
| | | cells, survival and persistence in the gut. |
| 33 | 95 | dTDP-4-keto-L. rhamnose reductase involved in |
| | | polysaccharide biosynthesis. Polysaccharides are important for |
| | | adhesion to gut cells, immune system modulation, stress |
| | | tolerance and for physical properties of fermented products. It |
| | | is used to identify or modify polysaccharide production and |
| | | interaction with gut cells. |
| 34 | 96 | Glucose inhibited division protein. Involved in stress |
| | | resistance, gidA mutants are UV-sensitive and exhibit |
| | | decreased homologous recombination in plasmidic tests. It is |
| | | used to identify or modify cell survival and gene regulation. |
| 35 | 97 | Glucose-1-phosphate thymidylyl transferase, involved in |
| | | polysaccharide biosynthesis. Polysaccharides are important for |
| | | adhesion to gut cells, immune system modulation, stress |
| | | tolerance and for physical properties of fermented products. It |
| | | is used to identify or modify polysaccharide production and |
| | | interaction with gut cells. |
| 36 | 98 | Phosphate starvation-induced protein, may be important for |
| | | survival under low phospate conditions. Phosphate levels have |
| | | been shown to be important in multistress resistance. It is used |
| | | to identify or modify cell survival. |
| 37 | 99 | Formate C-acetyltransferase (or pyruvate formate lyase, EC |
| | | 2.3.1.54). Converts formate to pyruvate during malate |
| | | utilization. Pyruvate is central to cell metabolism. It is used to |
| | | identify or modify metabolism and the generation of flavor |
| | | compounds. |
| 38 | 100 | Alpha-glycerophosphate oxidase. Oxidizes alpha- |
| | | glycerophosphate to dihydroxyacetone phosphate while |
| | | reducing oxygen to hydrogen peroxide. These compounds are |
| | | important for metabolism as well as antimicrobial activity. It is |
| | | used to identify or modify metabolism and the generation of |
| | | flavor compounds as well as antimicrobial activity. |
| 39 | 101 | 6-Phosphogluconate dehydrogenase. Converts 6-phospho-D- |
| | | gluconate to D-ribulose 5-phosphate and CO2, part of the |
| | | hexose monophosphate shunt pathway used for carbohydrate |
| | | metabolism. It is used to identify or modify metabolism and the |
| | | generation of flavor compounds. |
| 40 | 102 | 5-methyltetrahydropteroyltriglutamate homocysteine |
| | | methyltransferase. Converts 5-methyltetrahydropteroyltri-L- |
| | | glutamate and L-homocysteine toTetrahydropteroyltri-L- |
| | | glutamate and L-methionine. Sulpher compounds are important |
| | | in flavor development. Homocysteine is important in |
| | | cardiovascular health. It is used to identify or modify |
| | | metabolism and the generation of flavor or aroma compounds |
| | | as well as cardiovascular health. |
| 41 | 103 | S-methylmethionine permease. Integral membrane protein |
| | | involved in S-methylmethionine uptake. Sulfur compounds are |
| | | important in flavor development, and S-methylmethionine may |
| | | also be involved in cellular methylation pathways. Cellular |
| | | methylation is important for gene regulation. It is used to |
| | | identify or modify metabolism and the generation of flavor |
| | | compounds and for cellular methylation. |
| 42 | 104 | 6-Phospho-beta-galactosidase. Central to lactose metabolism, |
| | | results in alcohol compounds that may have flavor properties. It |
| | | is used to identify or modify metabolism and the generation of |
| | | flavor compounds. |
| 43 | 105 | GTP binding protein, membrane bound. Involved in the stress |
| | | response. It is used to identify or modify cell survival. |
| 44 | 106 | Gamma-glutamyl phosphate reductase (glutamate-5- |
| | | semialdehyde dehydrogenase), involved in proline biosynthesis |
| | | and amino acid metabolism pathways. It is used to identify or |
| | | modify metabolism and the generation of flavor compounds. |
| 45 | 107 | Dihydrofolate reductase (EC 1.5.1.3), responsible for resistance |
| | | to the cytotoxic drug methotrexate and involved in vitamin |
| | | synthesis. It is used to identify or modify metabolism and the |
| | | generation of vitamin compounds and for drug resistance. |
| 46 | 108 | Lactate dehydrogenase. Converts lactate to pyruvate, also has a |
| | | role in acid tolerance. Lactate can have antimicrobial effects. It |
| | | is used to identify or modify metabolism and the generation of |
| | | flavor compounds, for cell survival and virulence and |
| | | antimicrobial effects. |
| 47 | 109 | Heat-inducible transcription repressor protein. Involved in |
| | | stress resistance. It is used to identify or modify survival and on |
| | | gene regulation. |
| 48 | 110 | Daunorubicin resistance protein (DrrC) is a daunorubicin |
| | | resistance protein with a strong sequence similarity to the UvrA |
| | | protein that is involved in excision repair of DNA. DrrC is |
| | | induced by the anticancer drug daunorubicin and behaves like |
| | | an ATP-dependent, DNA binding protein in vitro. |
| 49 | 111 | Dihydrodipicolinate synthase (ec 4.2.1.52) (DHDPS) is also |
| | | known as DapA or AF0910. DapA catalyzes the first step in the |
| | | biosynthesis of diaminopimelate and lysine from aspartate |
| | | semialdehyde. The known pathways for diaminopimelate |
| | | (DAP) and lysine biosynthesis share two key enzymes, |
| | | dihydrodipicolinate synthase and dihydrodipicolinate reductase, |
| | | encoded by the dapA and dapB genes, respectively. |
| | | Diaminopimelate (DAP) is a metabolite that is also involved in |
| | | peptidoglycan formation. DapA can be used for the industrial |
| | | production of L-lysine. DHDPS belongs to the DHDPS family. |
| 50 | 112 | Lysin (Lys) is one of the lytic enzymes encoded by |
| | | bacteriophages. Together with holin, lysis of bacteria used in |
| | | cheese-making can be achieved to accelerate cheese ripening |
| | | and to facilitated release of intracellular enzymes involvement |
| | | in flavor formation. Production of holin alone leads to partial |
| | | lysis of the host cells, whereas production of lysin alone does |
| | | not cause significant lysis. Model cheese experiments in which |
| | | an inducible holinlysin overproducing strain was used showed a |
| | | fourfold increase in release of L-Lactate dehydrogenase activity |
| | | into the curd relative to the control strain and the holin- |
| | | overproducing strain, demonstrating the suitability of the |
| | | system for cheese applications. |
| 51 | 113 | Penicillin-binding protein 1A or PDPF is penicillin-binding |
| | | protein PBP 1A that is an essential murein polymerases of |
| | | bacteria. The penicillin binding proteins (PBPs) synthesize and |
| | | remodel peptidoglycan, the structural component of the |
| | | bacterial cell wall. Resistance to beta-lactam antibiotics in |
| | | bacteria is due to alteration of the penicillin-binding proteins |
| | | (PBPs). PBP 1A belongs to the class A high-molecular-mass |
| | | PBPs, which harbor transpeptidase (TP) and |
| | | glycosyltransferase (GT) activities. The GT active site |
| | | represents a target for the generation of novel non-penicillin |
| | | antibiotics. |
| 52 | 114 | Virulence-associated protein BH6253 plays a role in the |
| | | virulence of the pathogens. |
| 53 | 115 | Adherence and virulence protein A (Pav A) is a virulence factor |
| | | that is widely distributed in bacteria and participates in |
| | | adherence to host cells and soft tissue pathology. |
| 54 | 116 | Proline iminopeptidase gene (pepI) is part of an operon-like |
| | | structure of three open reading frames (ORF1, ORF2 and |
| | | ORF3). ORF1 was preceded by a typical prokaryotic promoter |
| | | region, and a putative transcription terminator was found |
| | | downstream of ORF3, identified as the pepI gene. PepI was |
| | | shown to be a metal-independent serine peptidase having thiol |
| | | groups at or near the active site. Kinetic studies identified |
| | | proline-p-nitroanilide as substrate. PepI is a dimer of M(r) |
| | | 53,000. The enzyme can be utilized to facilitate the |
| | | accumulation of proline from dipeptides and oligopeptides |
| | | during the ripening of cheese. |
| 55 | 117 | Sensory transduction protein regX3 forms part of a two- |
| | | component regulatory system regX3/senX3 phosphorylated by |
| | | senX3. The N-terminal region is similar to that of other |
| | | regulatory components of sensory transduction systems. The |
| | | senX3-regX3 IR contains a novel type of repetitive sequence, |
| | | called mycobacterial interspersed repetitive units (MIRUs). The |
| | | regX3 gene has utility in diagnostic assays to differentiate |
| | | between bacterial strains. |
| 56 | 118 | Aminopeptidase pepS (ec 3.4.11.—) is part of the proteolytic |
| | | system of lactic acid bacteria that is essential for bacterial |
| | | growth in milk and for development of the organoleptic |
| | | properties of dairy products. PepS is a monomeric |
| | | metallopeptidase of approximately 45 kDa with optimal |
| | | activity in the range pH 7.5-8.5 and at 55 degrees C on Arg- |
| | | paranitroanilide as substrate. PepS exhibits a high specificity |
| | | towards peptides possessing arginine or aromatic amino acids |
| | | at the N-terminus. PepS is part of the aminopeptidase T |
| | | family. In view of its substrate specificity, PepS is involved |
| | | both in bacterial growth by supplying amino acids, and in the |
| | | development of dairy products' flavor, by hydrolysing bitter |
| | | peptides and liberating aromatic amino acids which are |
| | | important precursors of aroma compounds. |
| 57 | 119 | Phosphoribosylaminoimidazolecarboxamide |
| | | formyltransferase/imp cyclohydrolase (ec 2.1.2.3) (purH) or |
| | | AICARFT is biosynthetic enzyme in the de novo purine |
| | | biosynthesis pathway. |
| 58 | 120 | Prolinase (pepR) is a peptidase gene expressing L-proline-beta- |
| | | naphthylamide-hydrolyzing activity. PepR was shown to be |
| | | the primary enzyme capable of hydrolyzing Pro-Leu in |
| | | Lactobacilli. The purified enzyme hydrolyzed Pro-Met, Thr- |
| | | Leu, and Ser-Phe as well as dipeptides containing neutral, |
| | | nonpolar amino acid residues at the amino terminus. Purified |
| | | pepR was determined to have a molecular mass of 125 kDa |
| | | with subunits of 33 kDa. The isoelectric point of the enzyme |
| | | was determined to be 4.5. PepR is a serine-dependent protease |
| | | that can be utilized in production of dairy products where it is |
| | | used to acidify milk. |
| 59 | 121 | Hexulose-6-phosphate isomerase (ec 5.—.—.—) is also known as |
| | | HumpI or SGBU and is part of a sugar metabolic pathway |
| | | along with sgbh where it is involved in isomerization of D- |
| | | arabino-6-hexulose 3-phosphate to D-fructose 6-phosphate. |
| | | SGBU belongs to the HumpI family. |
| 60 | 122 | Succinyl-diaminopimelate desuccinylase encodes the DapE that |
| | | has utility as antibiotic target. |
| 61 | 123 | Transcriptional regulator (GntR family) is part of the GntR |
| | | family of DNA binding proteins that has a characteristic helix- |
| | | turn-helix motif. The motif interacts with DNA double helix |
| | | and recognizes specific base sequences. |
| 62 | 124 | Xaa-Pro dipeptidase (ec 3.4.13.9) is also known as X-Pro |
| | | dipeptidase, proline dipeptidase, prolidase, imidodipeptidase or |
| | | pepQ. PepQ is involved in the hydrolysis of Xaa-\|-Pro |
| | | dipeptides and also acts on aminoacyl-hydroxyproline analogs. |
| | | PepQ belongs to peptidase family M24b. PepQ can be utilized |
| | | in the production of cheese. |

Isolated polynucleotides of the present invention include the polynucleotides identified herein as SEQ ID NO: 1-62; isolated polynucleotides comprising a polynucleotide sequence selected from the group consisting of SEQ ID NO: 1-62; isolated polynucleotides comprising at least a specified number of contiguous residues (x-mers) of any of the polynucleotides identified as SEQ ID NO: 1-62; isolated polynucleotides comprising a polynucleotide sequence that is complementary to any of the above polynucleotides; isolated polynucleotides comprising a polynucleotide sequence that is a reverse sequence or a reverse complement of any of the above polynucleotides; antisense sequences corresponding to any of the above polynucleotides; and variants of any of the above polynucleotides, as that term is described in this specification.

The word “polynucleotide(s),” as used herein, means a single or double stranded polymer of deoxyribonucleotide or ribonucleotide bases and includes DNA and corresponding RNA molecules, including mRNA molecules, both sense and antisense strands of DNA and RNA molecules, and comprehends cDNA, genomic DNA and recombinant DNA, as well as wholly or partially synthesized polynucleotides. A polynucleotide of the present invention may be an entire gene, or any portion thereof. A gene is a DNA sequence which codes for a functional protein or RNA molecule. Operable antisense polynucleotides may comprise a fragment of the corresponding polynucleotide, and the definition of “polynucleotide” therefore includes all operable antisense fragments. Antisense polynucleotides and techniques involving antisense polynucleotides are well known in the art and are described, for example, in Robinson-Benion, et al., “Antisense techniques,” Methods in Enzymol. 254(23): 363-375, 1995; and Kawasaki, et al., Artific. Organs 20 (8): 836-848, 1996.

The definitions of the terms “complement,” “reverse complement,” and “reverse sequence,” as used herein, are best illustrated by the following examples. For the sequence 5′ AGGACC 3′, the complement, reverse complement, and reverse sequences are as follows:

| | |
|---|---|
| complement | 3′ TCCTGG 5′ |
| reverse complement | 3′ GGTCCT 5′ |
| reverse sequence | 5′ CCAGGA 3′ |

Preferably, sequences that are complements of a specifically recited polynucleotide sequence are complementary over the entire length of the specific polynucleotide sequence.

Identification of genomic DNA and heterologous species DNA can be accomplished by standard DNA/DNA hybridization techniques, under appropriately stringent conditions, using all or part of a DNA sequence as a probe to screen an appropriate library. Alternatively, PCR techniques using oligonucleotide primers that are designed based on known DNA and protein sequences can be used to amplify and identify other identical or similar DNA sequences. Synthetic DNA corresponding to the identified sequences or variants thereof may be produced by conventional synthesis methods. All of the polynucleotides described herein are isolated and purified, as those terms are commonly used in the art.

The polynucleotides identified as SEQ ID NO: 1-62 may contain open reading frames (“ORFs”), or partial open reading frames, encoding polypeptides. Polynucleotides identified as SEQ ID NO: 1-62 may also contain non-coding sequences such as promoters and terminators that may be useful as control elements. Additionally, open reading frames encoding polypeptides may be identified in extended or full-length sequences corresponding to the sequences set out as SEQ ID NO: 1-62. Open reading frames may be identified using techniques that are well known in the art. These techniques include, for example, analysis for the location of known start and stop codons, most likely reading frame identification based on codon frequencies, similarity to known bacterial expressed genes, etc. Suitable tools and software for ORF analysis include GeneWise (The Sanger Center, Wellcome Trust Genome Campus, Hinxton, Cambridge CB10 ISA, United Kingdom), Diogenes (Computational Biology Centers, University of Minnesota, Academic Health Center, UMHG Box 43 Minneapolis Minn. 55455), and GRAIL (Informatics Group, Oak Ridge National Laboratories, Oak Ridge, Tennessee, Tenn.). Open reading frames and portions of open reading frames may be identified in the polynucleotides of the present invention. Once a partial open reading frame is identified, the polynucleotide may be extended in the area of the partial open reading frame using techniques that are well known in the art until the polynucleotide for the full open reading frame is identified. Thus, polynucleotides and open reading frames encoding polypeptides may be identified using the polynucleotides of the present invention.

Once open reading frames are identified in the polynucleotides of the present invention, the open reading frames may be isolated and/or synthesized. Expressible genetic constructs comprising the open reading frames and suitable promoters, initiators, terminators, etc., which are well known in the art, may then be constructed. Such genetic constructs may be introduced into a host cell to express the polypeptide encoded by the open reading frame. Suitable host cells may include various prokaryotic and eukaryotic cells. In vitro expression of polypeptides is also possible, as well known in the art.

As used herein, the term “oligonucleotide” refers to a relatively short segment of a polynucleotide sequence, generally comprising between 6 and 60 nucleotides, and comprehends both probes for use in hybridization assays and primers for use in the amplification of DNA by polymerase chain reaction.

As used herein, the term “x-mer,” with reference to a specific value of “x,” refers to a polynucleotide comprising at least a specified number (“x”) of contiguous residues of any of the polynucleotides identified as SEQ ID NO: 1-62. The value of x may be from about 20 to about 600, depending upon the specific sequence.

In another aspect, the present invention provides isolated polypeptides encoded, or partially encoded, by the above polynucleotides. As used herein, the term “polypeptide” encompasses amino acid chains of any length, including full-length proteins, wherein the amino acid residues are linked by covalent peptide bonds. The term “polypeptide encoded by a polynucleotide” as used herein, includes polypeptides encoded by a polynucleotide which comprises an isolated polynucleotide sequence or variant provided herein. Polypeptides of the present invention may be naturally purified products, or may be produced partially or wholly using recombinant techniques. Such polypeptides may be glycosylated with bacterial, fungal, mammalian or other eukaryotic carbohydrates or may be non-glycosylated. In specific embodiments, polypeptides of the present invention include an amino acid sequence recited in SEQ ID NO: 63-124.

Polypeptides of the present invention may be produced recombinantly by inserting a polynucleotide that encodes the polypeptide into an expression vector and expressing the polypeptide in an appropriate host. Any of a variety of expression vectors known to those of ordinary skill in the art may be employed. Expression may be achieved in any appropriate host cell that has been transformed or transfected with an expression vector containing a polypeptide encoding a recombinant polypeptide. Suitable host cells include prokaryotes, yeast and higher eukaryotic cells. Preferably, the host cells employed are Escherichia coli, Lactococcus lactis, Lactobacillus, insect, yeast or a mammalian cell line such as COS or CHO. The polynucleotide(s) expressed in this manner may encode naturally occurring polypeptides, portions of naturally occurring polypeptides, or other variants thereof.

In a related aspect, polypeptides are provided that comprise at least a functional portion of a polypeptide having an amino acid sequence encoded by a polynucleotide of the present invention. As used herein, a “functional portion” of a polypeptide is that portion which contains the active site essential for affecting the function of the polypeptide, for example, the portion of the molecule that is capable of binding one or more reactants. The active site may be made up of separate portions present on one or more polypeptide chains and will generally exhibit high binding affinity.

Functional portions of a polypeptide may be identified by first preparing fragments of the polypeptide by either chemical or enzymatic digestion of the polypeptide, or by mutation analysis of the polynucleotide that encodes the polypeptide and subsequent expression of the resulting mutant polypeptides. The polypeptide fragments or mutant polypeptides are then tested to determine which portions retain biological activity, using, for example, the representative assays provided below.

Portions and other variants of the inventive polypeptides may be generated by synthetic or recombinant means. Synthetic polypeptides having fewer than about 100 amino acids, and generally fewer than about 50 amino acids, may be generated using techniques that are well known to those of ordinary skill in the art. For example, such polypeptides may be synthesized using any of the commercially available solid-phase techniques, such as the Merrifield solid-phase synthesis method, where amino acids are sequentially added to a growing amino acid chain (See Merrifield, J. Am. Chem. Soc. 85:2149-2154, 1963). Equipment for automated synthesis of polypeptides is commercially available from suppliers such as Perkin Elmer/Applied Biosystems, Inc. (Foster City, Calif.), and may be operated according to the manufacturer's instructions. Variants of a native polypeptide may be prepared using standard mutagenesis techniques, such as oligonucleotide-directed site-specific mutagensis (Kunkel, Proc. Natl. Acad. Sci. USA 82: 488-492, 1985). Sections of DNA sequences may also be removed using standard techniques to permit preparation of truncated polypeptides.

In general, the polypeptides disclosed herein are prepared in an isolated, substantially pure form. Preferably, the polypeptides are at least about 80% pure; more preferably at least about 90% pure; and most preferably at least about 99% pure.

As used herein, the term “variant” comprehends polynucleotide or polypeptide sequences different from the specifically identified sequences, wherein one or more nucleotides or amino acid residues is deleted, substituted, or added. Variants may be naturally occurring allelic variants, or non-naturally occurring variants. Variant polynucleotide or polypeptide sequences preferably exhibit at least 75%, more preferably at least 80%, more preferably yet at least 85%, more preferably at least 90%, and most preferably at least 95% identity to a sequence of the present invention. The percentage identity is determined by aligning the two sequences to be compared as described below, determining the number of identical residues in the aligned portion, dividing that number by the total number of residues in the inventive (queried) sequence, and multiplying the result by 100.

Polynucleotide and polypeptide sequences may be aligned, and the percentage of identical residues in a specified region may be determined against another polynucleotide or polypeptide, using computer algorithms that are publicly available. Two exemplary algorithms for aligning and identifying the similarity of polynucleotide sequences are the BLASTN and FASTA algorithms. Polynucleotides may also be analyzed using the BLASTX algorithm, which compares the six-frame conceptual translation products of a nucleotide query sequence (both strands) against a protein sequence database. The percentage identity of polypeptide sequences may be examined using the BLASTP algorithm. The BLASTN, BLASTX and BLASTP programs are available on the NCBI anonymous FTP server and from the National Center for Biotechnology Information (NCBI), National Library of Medicine, Building 38A, Room 8N805, Bethesda, Md. 20894, USA. The BLASTN algorithm Version 2.0.4 [Feb. 24, 19981, Version 2.0.6 [Sep. 16, 1998] and Version 2.0.11 [Jan. 20, 2000], set to the parameters described below, is preferred for use in the determination of polynucleotide variants according to the present invention. The BLASTP algorithm, set to the parameters described below, is preferred for use in the determination of polypeptide variants according to the present invention. The use of the BLAST family of algorithms, including BLASTN, BLASTP and BLASTX, is described at NCBI's website and in the publication of Altschul, et al., Nucleic Acids Res. 25:3389-3402, 1997.

The computer algorithm FASTA is available on the Internet and from the University of Virginia by contacting David Hudson, Vice Provost for Research, University of Virginia, P.O. Box 9025, Charlottesville, Va. 22906-9025, USA. FASTA Version 2.0u4 [February 1996], set to the default parameters described in the documentation and distributed with the algorithm, may be used in the determination of variants according to the present invention. The use of the FASTA algorithm is described in Pearson and Lipman, Proc. Natl. Acad. Sci. USA 85:2444-2448, 1988; and Pearson, Methods in Enzymol. 183: 63-98, 1990.

The following running parameters are preferred for determination of alignments and similarities using BLASTN that contribute to the E values and percentage identity for polynucleotide sequences: Unix running command: blastall -p blastn -d embldb -e 10-G0-E0-r 1-v 30-b 30-i queryseq -o results; the parameters are: -p Program Name [String]; -d Database [String]; -e Expectation value (E) [Real]; -G Cost to open a gap (zero invokes default behavior) [Integer]; -E Cost to extend a gap (zero invokes default behavior) [Integer]; -r Reward for a nucleotide match (BLASTN only) [Integer]; -v Number of one-line descriptions (V) [Integer]; -b Number of alignments to show (B) [Integer]; -i Query File [File In]; and -o BLAST report Output File [File Out] Optional.

The following running parameters are preferred for determination of alignments and similarities using BLASTP that contribute to the E values and percentage identity of polypeptide sequences: blastall -p blastp -d swissprotdb -e 10-G 0-E 0-v 30-b 30-i queryseq -o results; the parameters are: -p Program Name [String]; -d Database [String]; -e Expectation value (E) [Real]; -G Cost to open a gap (zero invokes default behavior) [Integer]; -E Cost to extend a gap (zero invokes default behavior) [Integer]; -v Number of one-line descriptions (v) [Integer]; -b Number of alignments to show (b) [Integer]; -I Query File [File In]; -o BLAST report Output File [File Out] Optional. The “hits” to one or more database sequences by a queried sequence produced by BLASTN, FASTA, BLASTP or a similar algorithm, align and identify similar portions of sequences. The hits are arranged in order of the degree of similarity and the length of sequence overlap. Hits to a database sequence generally represent an overlap over only a fraction of the sequence length of the queried sequence.

The BLASTN, FASTA, and BLASTP algorithms also produce “Expect” values for alignments. The Expect value (E) indicates the number of hits one can “expect” to see over a certain number of contiguous sequences by chance when searching a database of a certain size. The Expect value is used as a significance threshold for determining whether the hit to a database, such as the preferred EMBL database, indicates true similarity. For example, an E value of 0.1 assigned to a polynucleotide hit is interpreted as meaning that in a database of the size of the EMBL database, one might expect to see 0.1 matches over the aligned portion of the sequence with a similar score simply by chance. By this criterion, the aligned and matched portions of the polynucleotide sequences then have a probability of 90% of being the same. For sequences having an E value of 0.01 or less over aligned and matched portions, the probability of finding a match by chance in the EMBL database is 1% or less using the BLASTN or FASTA algorithm.

According to one embodiment, “variant” polynucleotides and polypeptides, with reference to each of the polynucleotides and polypeptides of the present invention, preferably comprise sequences producing an E value of 0.01 or less when compared to the polynucleotide or polypeptide of the present invention. That is, a variant polynucleotide or polypeptide is any sequence that has at least a 99% probability of being the same as the polynucleotide or polypeptide of the present invention, measured as having an E value of 0.01 or less using the BLASTN, FASTA, or BLASTP algorithms set at parameters described above. According to a preferred embodiment, a variant polynucleotide is a sequence having the same number or fewer nucleic acids than a polynucleotide of the present invention that has at least a 99% probability of being the same as the polynucleotide of the present invention, measured as having an E value of 0.01 or less using the BLASTN or FASTA algorithms set at parameters described above. Similarly, according to a preferred embodiment, a variant polypeptide is a sequence having the same number or fewer amino acids than a polypeptide of the present invention that has at least a 99% probability of being the same as a polypeptide of the present invention, measured as having an E value of 0.01 or less using the BLASTP algorithm set at the parameters described above.

As noted above, the percentage identity is determined by aligning sequences using one of the BLASTN, FASTA, or BLASTP algorithms, set at the running parameters described above, and identifying the number of identical nucleic or amino acids over the aligned portions; dividing the number of identical nucleic or amino acids by the total number of nucleic or amino acids of the polynucleotide or polypeptide sequence of the present invention; and then multiplying by 100 to determine the percentage identity. For example, a polynucleotide of the present invention having 220 nucleic acids has a hit to a polynucleotide sequence in the EMBL database having 520 nucleic acids over a stretch of 23 nucleotides in the alignment produced by the BLASTN algorithm using the parameters described above. The 23 nucleotide hit includes 21 identical nucleotides, one gap and one different nucleotide. The percentage identity of the polynucleotide of the present invention to the hit in the EMBL library is thus 21/220 times 100, or 9.5%. The polynucleotide sequence in the EMBL database is thus not a variant of a polynucleotide of the present invention.

In addition to having a specified percentage identity to an inventive polynucleotide or polypeptide sequence, variant polynucleotides and polypeptides preferably have additional structure and/or functional features in common with the inventive polynucleotide or polypeptide. Polypeptides having a specified degree of identity to a polypeptide of the present invention share a high degree of similarity in their primary structure and have substantially similar functional properties. In addition to sharing a high degree of similarity in their primary structure to polynucleotides of the present invention, polynucleotides having a specified degree of identity to, or capable of hybridizing to an inventive polynucleotide preferably have at least one of the following features: (i) they contain an open reading frame or partial open reading frame encoding a polypeptide having substantially the same functional properties as the polypeptide encoded by the inventive polynucleotide; or (ii) they contain identifiable domains in common.

Alternatively, variant polynucleotides of the present invention hybridize to the polynucleotide sequences recited in SEQ ID NO: 1-62, or complements, reverse sequences, or reverse complements of those sequences, under stringent conditions. As used herein, “stringent conditions” refers to prewashing in a solution of 6×SSC, 0.2% SDS; hybridizing at 65° C., 6×SSC, 0.2% SDS overnight followed by two washes of 30 minutes each in 1×SSC, 0.1% SDS at 65° C and two washes of 30 minutes each in 0.2×SSC, 0.1% SDS at 65° C.

The present invention also encompasses polynucleotides that differ from the disclosed sequences but that, as a consequence of the discrepancy of the genetic code, encode a polypeptide having similar enzymatic activity as a polypeptide encoded by a polynucleotide of the present invention. Thus, polynucleotides comprising sequences that differ from the polynucleotide sequences recited in SEQ ID NO: 1-62, or complements, reverse sequences or reverse complements of those sequences, as a result of conservative substitutions are encompassed within the present invention. Additionally, polynucleotides comprising sequences that differ from the inventive polynucleotide sequences or complements, reverse complements or reverse sequences thereof, as a result of deletions and/or insertions totaling less than 10% of the total sequence length are also contemplated by and encompassed within the present invention. Similarly, polypeptides comprising sequences that differ from the inventive polypeptide sequences as a result of amino acid substitutions, insertions, and/or deletions totaling less than 10% of the total sequence length are contemplated by and encompassed within the present invention, provided the variant polypeptide has substantially the same functional activity to the inventive polypeptide.

The polynucleotides of the present invention may be isolated from various libraries, or may be synthesized using techniques that are well known in the art. The polynucleotides may be synthesized, for example, using automated oligonucleotide synthesizers (e.g., Beckman Oligo 1000M DNA Synthesizer) to obtain polynucleotide segments of up to 50 or more nucleic acids. A plurality of such polynucleotide segments may then be ligated using standard DNA manipulation techniques that are well known in the art of molecular biology. One conventional and exemplary polynucleotide synthesis technique involves synthesis of a single stranded polynucleotide segment having, for example, 80 nucleic acids, and hybridizing that segment to a synthesized complementary 85 nucleic acid segment to produce a 5-nucleotide overhang. The next segment may then be synthesized in a similar fashion, with a 5-nucleotide overhang on the opposite strand. The “sticky” ends ensure proper ligation when the two portions are hybridized. In this way, a complete polynucleotide of the present invention may be synthesized entirely in vitro.

Certain of the polynucleotides identified as SEQ ID NO: 1-62 are referred to as “partial” sequences, in that they may not represent the full coding portion of a gene encoding a naturally occurring polypeptide. The partial polynucleotide sequences disclosed herein may be employed to obtain the corresponding full-length genes for various species and organisms by, for example, screening DNA expression libraries using hybridization probes based on the polynucleotides of the present invention, or using PCR amplification with primers based upon the polynucleotides of the present invention. In this way one can, using methods well known in the art, extend a polynucleotide of the present invention upstream and downstream of the corresponding DNA, as well as identify the corresponding mRNA and genomic DNA, including the promoter and enhancer regions, of the complete gene. The present invention thus comprehends isolated polynucleotides comprising a sequence identified in SEQ ID NO: 1-62, or a variant of one of the specified sequences, that encode a functional polypeptide, including full length genes. Such extended polynucleotides may have a length of from about 50 to about 4,000 nucleic acids or base pairs, and preferably have a length of less than about 4,000 nucleic acids or base pairs, more preferably yet a length of less than about 3,000 nucleic acids or base pairs, most preferably yet a length of less than about 2,000 nucleic acids or base pairs. Under some circumstances, extended polynucleotides of the present invention may have a length of less than about 1,800 nucleic acids or base pairs, preferably less than about 1,600 nucleic acids or base pairs, more preferably less than about 1,400 nucleic acids or base pairs, more preferably yet less than about 1,200 nucleic acids or base pairs, and most preferably less than about 1,000 nucleic acids or base pairs.

Polynucleotides of the present invention comprehend polynucleotides comprising at least a specified number of contiguous residues (x-mers) of any of the polynucleotides identified as SEQ ID NO: 1-62 or their variants. According to preferred embodiments, the value of x is preferably at least 20, more preferably at least 40, more preferably yet at least 60, and most preferably at least 80. Thus, polynucleotides of the present invention include polynucleotides comprising a 20-mer, a 40-mer, a 60-mer, an 80-mer, a 100-mer, a 120-mer, a 150-mer, a 180-mer, a 220-mer a 250-mer, or a 300-mer, 400-mer, 500-mer or 600-mer of a polynucleotide identified as SEQ ID NO: 1-62 or a variant of one of the polynucleotides identified as SEQ ID NO: 1-62.

Oligonucleotide probes and primers complementary to and/or corresponding to SEQ ID NO: 1-62, and variants of those sequences, are also comprehended by the present invention. Such oligonucleotide probes and primers are substantially complementary to the polynucleotide of interest. An oligonucleotide probe or primer is described as “corresponding to” a polynucleotide of the present invention, including one of the sequences set out as SEQ ID NO: 1-62 or a variant thereof, if the oligonucleotide probe or primer, or its complement, is contained within one of the sequences set out as SEQ ID NO: 1-62 or a variant of one of the specified sequences.

Two single stranded sequences are said to be substantially complementary when the nucleotides of one strand, optimally aligned and compared, with the appropriate nucleotide insertions and/or deletions, pair with at least 80%, preferably at least 90% to 95%, and more preferably at least 98% to 100%, of the nucleotides of the other strand. Alternatively, substantial complementarity exists when a first DNA strand will selectively hybridize to a second DNA strand under stringent hybridization conditions. Stringent hybridization conditions for determining complementarity include salt conditions of less than about 1 M, more usually less than about 500 mM and preferably less than about 200 mM. Hybridization temperatures can be as low as 5° C., but are generally greater than about 22° C., more preferably greater than about 30° C. and most preferably greater than about 37° C. Longer DNA fragments may require higher hybridization temperatures for specific hybridization. Since the stringency of hybridization may be affected by other factors such as probe composition, presence of organic solvents and extent of base mismatching, the combination of parameters is more important than the absolute measure of any one alone. DNA-DNA hybridization studies may performed using either genomic DNA or DNA derived by preparing cDNA from the RNA present in a sample to be tested.

In addition to DNA-DNA hybridization, DNA-RNA or RNA-RNA hybridization assays are also possible. In the first case, the mRNA from expressed genes would then be detected instead of genomic DNA or cDNA derived from mRNA of the sample. In the second case, RNA probes could be used. In addition, artificial analogs of DNA hybridizing specifically to target sequences could also be used.

In specific embodiments, the oligonucleotide probes and/or primers comprise at least about 6 contiguous residues, more preferably at least about 10 contiguous residues, and most preferably at least about 20 contiguous residues complementary to a polynucleotide sequence of the present invention. Probes and primers of the present invention may be from about 8 to 100 base pairs in length, preferably from about 10 to 50 base pairs in length or, more preferably, from about 15 to 40 base pairs in length. The primers and probes may be readily selected using procedures well known in the art, taking into account DNA-DNA hybridization stringencies, annealing and melting temperatures, potential for formation of loops and other factors, which are well known in the art. Tools and software suitable for designing probes, and especially suitable for designing PCR primers, are available on the Internet. In addition, a software program suitable for designing probes, and especially for designing PCR primers, is available from Premier Biosoft International, 3786 Corina Way, Palo Alto, Calif. 94303-4504. Preferred techniques for designing PCR primers are also disclosed in Dieffenbach and Dyksler, PCR primer: a laboratory manual, CSHL Press: Cold Spring Harbor, N.Y., 1995.

A plurality of oligonucleotide probes or primers corresponding to a polynucleotide of the present invention may be provided in a kit form. Such kits generally comprise multiple DNA or oligonucleotide probes, each probe being specific for a polynucleotide sequence. Kits of the present invention may comprise one or more probes or primers corresponding to a polynucleotide of the present invention, including a polynucleotide sequence identified in SEQ ID NO: 1-62.

In one embodiment useful for high-throughput assays, the oligonucleotide probe kits of the present invention comprise multiple probes in an array format, wherein each probe is immobilized in a predefined, spatially addressable location on the surface of a solid substrate. Array formats which may be usefully employed in the present invention are disclosed, for example, in U.S. Pat. No. 5,412,087, 5,545,531 and PCT Publication No. WO 95/00530, the disclosures of which are hereby incorporated by reference.

Oligonucleotide probes for use in the present invention may be constructed synthetically prior to immobilization on an array, using techniques well known in the art (See, for example, Gait, ed., Oligonucleotide synthesis a practical approach, RL Press: Oxford, England, 1984). Automated equipment for the synthesis of oligonucleotides is available commercially from such companies as Perkin Elmer/Applied Biosystems Division (Foster City, Calif.) and may be operated according to the manufacturer's instructions. Alternatively, the probes may be constructed directly on the surface of the array using techniques taught, for example, in PCT Publication No. WO 95/00530.

The solid substrate and the surface thereof preferably form a rigid support and are generally formed from the same material. Examples of materials from which the solid substrate may be constructed include polymers, plastics, resins, membranes, polysaccharides, silica or silica-based materials, carbon, metals and inorganic glasses. Synthetically prepared probes may be immobilized on the surface of the solid substrate using techniques well known in the art, such as those disclosed in U.S. Pat. No. 5,412,087.

In one such technique, compounds having protected functional groups, such as thiols protected with photochemically removable protecting groups, are attached to the surface of the substrate. Selected regions of the surface are then irradiated with a light source, preferably a laser, to provide reactive thiol groups. This irradiation step is generally performed using a mask having apertures at predefined locations using photolithographic techniques well known in the art of semiconductors. The reactive thiol groups are then incubated with the oligonucleotide probe to be immobilized. The precise conditions for incubation, such as temperature, time and pH, depend on the specific probe and can be easily determined by one of skill in the art. The surface of the substrate is washed free of unbound probe and the irradiation step is repeated using a second mask having a different pattern of apertures. The surface is subsequently incubated with a second, different, probe. Each oligonucleotide probe is typically immobilized in a discrete area of less than about 1 mm². Preferably each discrete area is less than about 10,000 mm², more preferably less than about 100 mm². In this manner, a multitude of oligonucleotide probes may be immobilized at predefined locations on the array.

The resulting array may be employed to screen for differences in organisms or samples or products containing genetic material as follows. Genomic or cDNA libraries are prepared using techniques well known in the art. The resulting target DNA is then labeled with a suitable marker, such as a radiolabel, chromophore, fluorophore or chemiluminescent agent, using protocols well known for those skilled in the art. A solution of the labeled target DNA is contacted with the surface of the array and incubated for a suitable period of time.

The surface of the array is then washed free of unbound target DNA and the probes to which the target DNA hybridized are determined by identifying those regions of the array to which the markers are attached. When the marker is a radiolabel, such as ³²P, autoradiography is employed as the detection method. In one embodiment, the marker is a fluorophore, such as fluorescein, and the location of bound target DNA is determined by means of fluorescence spectroscopy. Automated equipment for use in fluorescence scanning of oligonucleotide probe arrays is available from Affymetrix, Inc. (Santa Clara, Calif.) and may be operated according to the manufacturer's instructions. Such equipment may be employed to determine the intensity of fluorescence at each predefined location on the array, thereby providing a measure of the amount of target DNA bound at each location. Such an assay would be able to indicate not only the absence and presence of the marker probe in the target, but also the quantitative amount as well.

The significance of such high-throughput screening system is apparent for applications such as microbial selection and quality control operations in which there is a need to identify large numbers of samples or products for unwanted materials, to identify microbes or samples or products containing microbial material for quarantine purposes, etc., or to ascertain the true origin of samples or products containing microbes. Screening for the presence or absence of polynucleotides of the present invention used as identifiers for tagging microbes and microbial products can be valuable for later detecting the genetic composition of food, fermentation and industrial microbes or microbes in human or animal digestive system after consumption of probiotics, etc.

In this manner, oligonucleotide probe kits of the present invention may be employed to examine the presence/absence (or relative amounts in case of mixtures) of polynucleotides in different samples or products containing different materials rapidly and in a cost-effective manner. Examples of microbial species which may be examined using the present invention, include lactic acid bacteria, such as Lactobacillus rhamnosus, and other microbial species.

Another aspect of the present invention involves collections of a plurality of polynucleotides of the present invention. A collection of a plurality of the polynucleotides of the present invention, particularly the polynucleotides identified as SEQ ID NO: 1-62, may be recorded and/or stored on a storage medium and subsequently accessed for purposes of analysis, comparison, etc. Suitable storage media include magnetic media such as magnetic diskettes, magnetic tapes, CD-ROM storage media, optical storage media, and the like. Suitable storage media and methods for recording and storing information, as well as accessing information such as polynucleotide sequences recorded on such media, are well known in the art. The polynucleotide information stored on the storage medium is preferably computer-readable and may be used for analysis and comparison of the polynucleotide information.

Another aspect of the present invention thus involves storage medium on which are recorded a collection of the polynucleotides of the present invention, particularly a collection of the polynucleotides identified as SEQ ID NO: 1-62. According to one embodiment, the storage medium includes a collection of at least 20, preferably at least 50, more preferably at least 100, and most preferably at least 200 of the polynucleotides of the present invention, preferably the polynucleotides identified as SEQ ID NO: 1-62, including variants of those polynucleotides.

Another aspect of the present invention involves a combination of polynucleotides, the combination containing at least 5, preferably at least 10, more preferably at least 20, and most preferably at least 50 different polynucleotides of the present invention, including polynucleotides selected from SEQ ID NO: 1-62, and variants of these polynucleotides.

In another aspect, the present invention provides genetic constructs comprising, in the 5′-3′ direction, a gene promoter sequence and an open reading frame coding for at least a functional portion of a polypeptide encoded by a polynucleotide of the present invention. In certain embodiments, the genetic constructs of the present invention also comprise a gene termination sequence. The open reading frame may be oriented in either a sense or antisense direction. Genetic constructs comprising a non-coding region of a gene coding for a polypeptide encoded by the above polynucleotides or a nucleotide sequence complementary to a non-coding region, together with a gene promoter sequence, are also provided. A terminator sequence may form part of this construct. Preferably, the gene promoter and termination sequences are functional in a host organism. More preferably, the gene promoter and termination sequences are common to those of the polynucleotide being introduced. The genetic construct may further include a marker for the identification of transformed cells.

Techniques for operatively linking the components of the genetic constructs are well known in the art and include the use of synthetic linkers containing one or more restriction endonuclease sites as described, for example, by Sambrook et al., in Molecular cloning: a laboratory manual, Cold Spring Harbor Laboratories Press: Cold Spring Harbor, N.Y., 1989. The genetic constructs of the present invention may be linked to a vector having at least one replication system, for example, E. coli, whereby after each manipulation, the resulting construct can be cloned and sequenced and the correctness of the manipulation determined.

Transgenic microbial cells comprising the genetic constructs of the present invention are also provided by the present invention, together with microbes comprising such transgenic cells, products and progeny of such microbes, and materials including such microbes. Techniques for stably incorporating genetic constructs into the genome of target microbes, such as Lactobacillus species, Lactococcus lactis or E. coli, are well known in the art of bacterial transformation and are exemplified by the transformation of E. coli for sequencing in Example 1.

Transgenic, non-microbial, cells comprising the genetic constructs of the present invention are also provided, together with organisms comprising such transgenic cells, and products and progeny of such organisms. Genetic constructs of the present invention may be stably incorporated into the genomes of non-microbial target organisms, such as fungi, using techniques well known in the art.

In preferred embodiments, the genetic constructs of the present invention are employed to transform microbes used in the production of food products, ingredients, processing aids, additives or supplements and for the production of microbial products for pharmaceutical uses, particularly for modulating immune system function and immunological effects, and in the production of chemoprotectants providing beneficial effects, probiotics and health supplements. The inventive genetic constructs may also be employed to transform bacteria that are used to produce enzymes or substances such as polysaccharides, flavor compounds, and bioactive substances, and to enhance resistance to industrial processes such as drying and to adverse stimuli in the human digestive system. The genes involved in antibiotic production, and phage uptake and resistance in Lactobacillus rhamnosus are considered to be especially useful. The target microbe to be used for transformation with one or more polynucleotides or genetic constructs of the present invention is preferably selected from the group consisting of bacterial genera Lactococcus, Lactobacillus, Streptococcus, Oenococcus, Lactosphaera, Trichococcus, Pediococcus and others potentially useful in various fermentation industries selected, most preferably, from the group consisting of Lactobacillus species in the following list: Lactobacillus acetotolerans, Lactobacillus acidophilus, Lactobacillus agilis, Lactobacillus alimentarius, Lactobacillus amylolyticus, Lactobacillus amylophilus, Lactobacillus amylovorus, Lactobacillus animalis, Lactobacillus arizonae, Lactobacillus aviarius, Lactobacillus bavaricus, Lactobacillus bifermentans, Lactobacillus brevis, Lactobacillus buchneri, Lactobacillus bulgaricus, Lactobacillus casei, Lactobacillus collinoides, Lactobacillus coryniformis, Lactobacillus crispatus, Lactobacillus curvatus, Lactobacillus delbrueckii, Lactobacillus delbrueckii subsp. bulgaricus, Lactobacillus delbrueckii subsp. lactis, Lactobacillus farciminis, Lactobacillus fermentum, Lactobacillus fructivorans, Lactobacillus gallinarum, Lactobacillus gasseri, Lactobacillus graminis, Lactobacillus hamsteri, Lactobacillus helveticus, Lactobacillus helveticus subsp. jugurti, Lactobacillus hetero, Lactobacillus hilgardii, Lactobacillus homohiochii, Lactobacillus japonicus, Lactobacillus johnsonii, Lactobacillus kefiri, Lactobacillus lactis, Lactobacillus leichmannii, Lactobacillus lindneri, Lactobacillus mali, Lactobacillus maltaromicus, Lactobacillus manihotivorans, Lactobacillus mucosae, Lactobacillus murinus, Lactobacillus oris, Lactobacillus panis, Lactobacillus paracasei, Lactobacillus paracasei subsp. pseudoplantarum, Lactobacillus paraplantarum, Lactobacillus pentosus, Lactobacillus plantarum, Lactobacillus pontis, Lactobacillus reuteri, Lactobacillus rhamnosus, Lactobacillus ruminis, Lactobacillus sake, Lactobacillus salivarius, Lactobacillus salivarius subsp. salicinius, Lactobacillus salivarius subsp. salivarius, Lactobacillus sanfranciscensis, Lactobacillus sharpeae, Lactobacillus thermophilus, Lactobacillus vaginalis, Lactobacillus vermiforme, Lactobacillus zeae.

In yet a further aspect, the present invention provides methods for modifying the concentration, composition and/or activity of a polypeptide in a host organism, such as a microbe, comprising stably incorporating a genetic construct of the present invention into the genome of the host organism by transforming the host organism with such a genetic construct. The genetic constructs of the present invention may be used to transform a variety of organisms.

Food products, ingredients, processing aids, additives and/or supplements comprising microbes transformed with the inventive genetic constructs are also provided, together with food products, ingredients, processing aids, additives and/or supplements prepared, or derived, from milk to which a polypeptide of the present invention has been added. Preferably such food products, ingredients, processing aids, additives and/or supplements have at least one enhanced property, such as improved flavor, aroma, texture, nutritional benefits, immune system modulation, and/or health benefits. Examples of such food products include, but are not limited to, cheese and yoghurt.

The polynucleotides of the present invention may be further employed as non-disruptive tags for marking organisms, particularly microbes. Other organisms may, however, be tagged with the polynucleotides of the present invention, including commercially valuable plants, animals, fish, fungi and yeasts. Genetic constructs comprising polynucleotides of the present invention may be stably introduced into an organism as heterologous, non-functional, non-disruptive tags. It is then possible to identify the origin or source of the organism at a later date by determining the presence or absence of the tag(s) in a sample of material. Detection of the tag(s) may be accomplished using a variety of conventional techniques, and will generally involve the use of nucleic acid probes. Sensitivity in assaying the presence of probe can be usefully increased by using branched oligonucleotides, as described by Horn et al., Nucleic Acids Res. 25(23):4842-4849, 1997, enabling detection of as few as 50 DNA molecules in the sample.

Polynucleotides of the present invention may also be used to specifically suppress gene expression by methods that operate post-transcriptionally to block the synthesis of products of targeted genes, such as RNA interference (RNAi), and quelling. For a review of techniques of gene suppression see Science, 288:1370-1372, 2000. Exemplary gene silencing methods are also provided in WO 99/49029 and WO 99/53050. Posttranscriptional gene silencing is brought about by a sequence-specific RNA degradation process which results in the rapid degradation of transcripts of sequence-related genes. Studies have provided evidence that double-stranded RNA may act as a mediator of sequence-specific gene silencing (see, e.g., review by Montgomery and Fire, Trends in Genetics, 14: 255-258, 1998). Gene constructs that produce transcripts with self-complementary regions are particularly efficient at gene silencing. A unique feature of this posttranscriptional gene silencing pathway is that silencing is not limited to the cells where it is initiated. The gene-silencing effects may be disseminated to other parts of an organism and even transmitted through the germ line to several generations.

The polynucleotides of the present invention may be employed to generate gene silencing constructs and or gene-specific self-complementary RNA sequences that can be delivered by conventional art-known methods to cells and tissues. Within genetic constructs, sense and antisense sequences can be placed in regions flanking an intron sequence in proper splicing orientation with donor and acceptor splicing sites, such that intron sequences are removed during processing of the transcript and sense and antisense sequences, as well as splice junction sequences, bind together to form double-stranded RNA. Alternatively, spacer sequences of various lengths may be employed to separate self-complementary regions of sequence in the construct. During processing of the gene construct transcript, intron sequences are spliced-out, allowing sense and anti-sense sequences, as well as splice junction sequences, to bind forming double-stranded RNA. Select ribonucleases bind to and cleave the double-stranded RNA, thereby initiating the cascade of events leading to degradation of specific mRNA gene sequences, and silencing specific genes. Alternatively, rather than using a gene construct to express the self-complementary RNA sequences, the gene-specific double-stranded RNA segments are delivered to one or more targeted areas to be internalized into the cell cytoplasm to exert a gene silencing effect. Gene silencing RNA sequences comprising the polynucleotides of the present invention are useful for creating genetically modified organisms with desired phenotypes as well as for characterizing genes (e.g., in high-throughput screening of sequences), and studying their functions in intact organisms.

In another aspect, the present invention provides methods for using one or more of the inventive polypeptides or polynucleotides to treat disorders in a mammal, such as a human.

In this aspect, the polypeptide or polynucleotide is generally present within a composition, such as a pharmaceutical or immunogenic composition. Pharmaceutical compositions may comprise one or more polypeptides, each of which may contain one or more of the above sequences (or variants thereof), and a physiologically acceptable carrier. Immunogenic compositions may comprise one or more of the above polypeptides and an immunostimulant, such as an adjuvant or a liposome, into which the polypeptide is incorporated.

Alternatively, a composition of the present invention may contain DNA encoding one or more polypeptides described herein, such that the polypeptide is generated in situ. In such compositions, the DNA may be present within any of a variety of delivery systems known to those of ordinary skill in the art, including nucleic acid expression systems, and bacterial and viral expression systems. Appropriate nucleic acid expression systems contain the necessary DNA sequences for expression in the patient (such as a suitable promoter and terminator signal). Bacterial delivery systems involve the administration of a bacterium (such as Bacillus Calmette-Guerin) that expresses an immunogenic portion of the polypeptide on its cell surface. In a preferred embodiment, the DNA may be introduced using a viral expression system (e.g., vaccinia or other poxvirus, retrovirus, or adenovirus), which may involve the use of a non-pathogenic, or defective, replication competent virus. Techniques for incorporating DNA into such expression systems are well known in the art. The DNA may also be “naked,” as described, for example, in Ulmer et al., Science 259:1745-1749, 1993 and reviewed by Cohen, Science 259:1691-1692, 1993. The uptake of naked DNA may be increased by coating the DNA onto biodegradable beads, which are efficiently transported into the cells.

While any suitable carrier known to those of ordinary skill in the art may be employed in the pharmaceutical compositions of this invention, the type of carrier will vary depending on the mode of administration. For parenteral administration, such as subcutaneous injection, the carrier preferably comprises water, saline, alcohol, a lipid, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactic galactide) may also be employed as carriers for the pharmaceutical compositions of this invention. Suitable biodegradable microspheres are disclosed, for example, in U.S. Pat. Nos. 4,897,268 and 5,075,109.

Any of a variety of adjuvants may be employed in the immunogenic compositions of the present invention to non-specifically enhance an immune response. Most adjuvants contain a substance designed to protect the antigen from rapid catabolism, such as aluminum hydroxide or mineral oil, and a non-specific stimulator of immune responses, such as lipid A, Bordetella pertussis or M. tuberculosis. Suitable adjuvants are commercially available as, for example, Freund's Incomplete Adjuvant and Freund's Complete Adjuvant (Difco Laboratories, Detroit, Mich.), and Merck Adjuvant 65 (Merck and Company, Inc., Rahway, N.J.). Other suitable adjuvants include alum, biodegradable microspheres, monophosphoryl lipid A and Quil A.

Routes and frequency of administration, as well as dosage, vary from individual to individual. In general, the inventive compositions may be administered by injection (e.g., intradermal, intramuscular, intravenous or subcutaneous), intranasally (e.g., by aspiration) or orally. In general, the amount of polypeptide present in a dose (or produced in situ by the DNA in a dose) ranges from about 1 pg to about 100 mg per kg of host, typically from about 10 pg to about 1 mg per kg of host, and preferably from about 100 pg to about 1 pg per kg of host. Suitable dose sizes will vary with the size of the patient, but will typically range from about 0.1 ml to about 2 ml.

The following examples are offered by way of illustration and not by way of limitation.

### EXAMPLE 1

### Isolation and Characterization of DNA Sequences from Lactobacillus rhamnosus Strain HN001

Lactobacillus rhamnosus strain HN001 DNA libraries were constructed and screened as follows.

DNA was prepared in large scale by cultivating the bacteria in 2×100 ml cultures with 100 ml MRS broth (Difco Laboratories, Detroit Mich.) and 1 ml Lactobacillus glycerol stock as inoculum, placed into 500 ml culture flasks and incubated at 37° C. for approx. 16 hours with shaking (220 rpm).

The cultures were centrifuged at 6200 rpm for 10 min to pellet the cells. The supernatant was removed and the cell pellet resuspended in 40 ml fresh MRS broth and transferred to clean 500 ml culture flasks. Fresh MRS broth (60 ml) was added to bring the volume back to 100 ml and flasks were incubated for a further 2 hrs at 37° C. with shaking (220 rpm). The cells were pelleted by centrifugation (6200 rpm for 10 min) and supernatant removed. Cell pellets were washed twice in 20 ml buffer A (50 mM NaCl, 30 mM Tris pH 8.0, 0.5 mM EDTA).

Cells were resuspended in 2.5 ml buffer B (25% sucrose (w/v), 50 mM Tris pH 8.0, 1 mM EDTA, 20 mg/ml lysozyme, 20 μg/ml mutanolysin) and incubated at 37° C. for 45 min. Equal volumes of EDTA (0.25 M) was added to each tube and allowed to incubate at room temperature for 5 min. 20% SDS (1 ml) solution was added, mixed and incubated at 65° C. for 90 min. 50 μl Proteinase K (Gibco BRL, Gaithersburg, Md.) from a stock solution of 20 mg/ml was added and tubes incubated at 65° C. for 15 min.

DNA was extracted with equal volumes of phenol:chloroform:isoamylalcohol (25:24:1). Tubes were centrifuged at 6200 rpm for 40 min. The aqueous phase was removed to clean sterile Oak Ridge centrifuge tubes (30 ml). Crude DNA was precipitated with an equal volume of cold isopropanol and incubated at −20° C. overnight.

After resuspension in 500 μl TE buffer, DNase-free RNase was added to a final concentraion of 100 μg/ml and incubated at 37° C. for 30 min. The incubation was extended for a further 30 min after adding 100 μl Proteinase K from a stock solution of 20 mg/ml. DNA was precipitated with ethanol after a phenol:chloroform:isoamylalcohol (25:24:1) and a chloroform:isoamylalcohol (24:1) extraction and dissolved in 250 μl TE buffer.

DNA was digested with Sau3AI at a concentration of 0.004 U/μg in a total volume of 1480 μl, with 996 μl DNA, 138.75 μl 10× REACT 4 buffer and 252.75 μl H₂O. Following incubation for 1 hour at 37° C., DNA was divided into two tubes. 31 μl 0.5 M EDTA was added to stop the digestion and 17 μl samples were taken for agarose gel analysis. Samples were put into 15 ml Falcon tubes and diluted to 3 ml for loading onto sucrose gradient tubes.

Sucrose gradient size fractionation was conducted as follows. 100 ml of 50% sucrose (w/v) was made in TEN buffer (1M NaCl, 20 mM Tris pH 8.0, 5 mM EDTA) and sterile filtered. Dilutions of 5, 10, 15, 20, 25, 30, 62 and 40% sucrose were prepared and overlaid carefully in Beckman Polyallomer tubes, and kept overnight at 4° C. TEN buffer (4 ml) was loaded onto the gradient, with 3 ml of DNA solution on top. The gradients were centrifuged at 26K for 18 hours at 4° C. in a Centricon T-2060 centrifuge using a Kontron TST 28-38 rotor. After deceleration without braking (approx. 1 hour), the gradients were removed and fractions collected using an auto Densi-Flow (Haake-Buchler Instruments). Agarose gel was used to analyse the fractions. The best two pairs of fractions were pooled and diluted to contain less than 10% sucrose. TEN buffer (4 ml) was added and DNA precipitated with 2 volumes of 100% ice cold ethanol and an overnight incubation at −20° C.

DNA pellets were resuspended in 300 μl TE buffer and re-precipitated for approx. 6 hours at −20° C. after adding 1/10 volume 3 M NaOAC pH 5.2 and 2 volumes of ethanol. DNA was pelleted at top speed in a microcentrifuge for 15 min, washed with 70% ethanol and pelleted again, dried and resuspended in 10 μl TE buffer.

DNA was ligated into dephosphorylated BamHI-digested pBluescript SK II⁺ and dephosphorylated BamHI-digested lambda ZAP Express using standard protocols. Packaging of the DNA was done using Gigapack III Gold packaging extract (Stratagene, La Jolla, Calif.) following the manufacturer's protocols. Packaged libraries were stored at 4° C.

Mass excision from the primary packaged phage library was done using XL1-Blue MRF′ cells and ExAssist Helper Phage (Stratagene). The excised phagemids were diluted with NZY broth (Gibco BRL, Gaithersburg, Md.) and plated out onto LB-kanamycin agar plates containing 5-bromo-4-chloro-3-indolyl-β-D-galactoside (X-gal) and isopropylthio-beta-galactoside (IPTG). After incubation, single colonies were picked for PCR size determination before the most suitable libraries were selected for sequencing.

Of the colonies picked for DNA minipreps and subsequent sequencing, the large majority contained an insert suitable for sequencing. Positive colonies were cultured in LB broth with kanamycin or ampicillin depending on the vector used, and DNA was purified by means of rapid alkaline lysis minipreps (solutions: Qiagen, Venlo, The Netherlands; clearing plates, Millipore, Bedford, Mass.). Agarose gels at 1% were used to screen sequencing templates for chromosomal contamination and concentration. Dye terminator sequencing reactions were prepared using a Biomek 2000 robot (Beckman Coulter, Inc., Fullerton, Calif.) and Hydra 96 (Robbins Scientific, Sunnyvale, Calif.) for liquid handling. DNA amplification was done in a 9700 PCR machine (Perkin Elmer/Applied Biosystems, Foster City, Calif.) according to the manufacturer's protocol.

The sequence of the genomic DNA fragments were determined using a Perkin Elmer/Applied Biosystems Division Prism 377 sequencer.

To extend the sequences of the inserts from these clones, primers were designed from the determined nucleotide sequences so that the primer sequences are located approximately 100 bp downstream of the 5′ end and 100 bp upstream of the 3′ end of the determined nucleotide sequence. Primers were selected using the Gap4 Genome Assembly Program (Bonfield et al., Nucleic Acids Res. 24:4992-4999, 1995) using the following parameters: No. of bases ahead: 40; No. of bases back: 40; Minimum melting temperature: 55° C.; maximum melting temperature: 60° C.; minimum length: 17 bp; maximum length: 20 bp; minimum GC-content: 40%; maximum GC-content: 60%. Sequencing of clones was performed as described above. The determined nucleotide sequences are identified as SEQ ID NO: 1-62 disclosed herein.

This example not only shows how the sequences were obtained, but also that a bacterium (E. coli) can be stably transformed with any desired DNA fragment of the present invention for permanent marking for stable inheritance.

The determined DNA sequences were compared to and aligned with known sequences in the public databases. Specifically, the polynucleotides identified in SEQ ID NO: 1-62 were compared to polynucleotides in the EMBL database as of the end of July 2000, using BLASTN algorithm Version 2.0.11 [Jan. 20, 2000], set to the following running parameters: Unix running command: blastall -p blastn -d embldb -e 10-G 0-E 0-r 1-v 30-b 30-i queryseq -o results. Multiple alignments of redundant sequences were used to build up reliable consensus sequences. Based on similarity to known sequences, the isolated polynucleotides of the present invention identified as SEQ ID NO: 1-62 were putatively identified as encoding polypeptides having similarity to the polypeptides shown above in Table 1. The amino acid sequences encoded by the DNA sequences of SEQ ID NO: 1-62 are provided in SEQ ID NO: 63-124, respectively.

Several of the sequences provided in SEQ ID NO: 1-62 were found to be full-length and to contain open reading frames (ORFs). Specifically, SEQ ID NO: 1, 2, 4-12, 14, 20, 21, 24, 26, 34, 36, 42, 44, 45, 54, 55, 59 and 61 were found to be full-length. The location of ORFs (by nucleotide position) contained within SEQ ID NO: 1-62, and the corresponding amino acid sequences are provided in Table 2 below.

TABLE 2

| Polynucleotide | | Polypeptide |
|---|---|---|
| SEQ ID NO: | Open reading frame | SEQ ID NO: |
| 1 | 1-672 | 63 |
| 2 | 1-1,419 | 64 |
| 3 | 1-1,104 | 65 |
| 4 | 1-1,107 | 66 |
| 5 | 1-1,170 | 67 |
| 6 | 1-891 | 68 |
| 7 | 1-1,170 | 69 |
| 8 | 1-1,158 | 70 |
| 9 | 1-786 | 71 |
| 10 | 1-927 | 72 |
| 11 | 1-810 | 73 |
| 12 | 1-1,422 | 74 |
| 13 | 1-768 | 75 |
| 14 | 1-1,923 | 76 |
| 15 | 1-1,443 | 77 |
| 16 | 1-993 | 78 |
| 17 | 1-1,032 | 79 |
| 18 | 1-1,674 | 80 |
| 19 | 1-876 | 81 |
| 20 | 1-732 | 82 |
| 21 | 1-1,299 | 83 |
| 22 | 1-1,344 | 84 |
| 23 | 1-474 | 85 |
| 24 | 1-1,002 | 86 |
| 25 | 1-1,239 | 87 |
| 26 | 1-1,881 | 88 |
| 27 | 1-606 | 89 |
| 28 | 1-1,023 | 90 |
| 29 | 1-1,227 | 91 |
| 30 | 1-1,158 | 92 |
| 31 | 1-1,071 | 93 |
| 32 | 1-1,308 | 94 |
| 33 | 1-645 | 95 |
| 34 | 1-1,920 | 96 |
| 35 | 1-762 | 97 |
| 36 | 1-936 | 98 |
| 37 | 1-840 | 99 |
| 38 | 1-1,341 | 100 |
| 39 | 1-726 | 101 |
| 40 | 1-972 | 102 |
| 41 | 1-888 | 103 |
| 42 | 1-1,422 | 104 |
| 43 | 1-774 | 105 |
| 44 | 1-1,254 | 106 |
| 45 | 1-489 | 107 |
| 46 | 1-285 | 108 |
| 47 | 1-969 | 109 |
| 48 | 417-1,336 | 110 |
| 49 | 1-760 | 111 |
| 50 | 193-846 | 112 |
| 51 | 463-1,310 | 113 |
| 52 | 628-1,662 | 114 |
| 53 | 1-887 | 115 |
| 54 | 251-946 | 116 |
| 55 | 66-743 | 117 |
| 56 | 1-780 | 118 |
| 57 | 256-1,569 | 119 |
| 58 | 274-1,112 | 120 |
| 59 | 8-954 | 121 |
| 60 | 17-948 | 122 |
| 61 | 206-1,006 | 123 |
| 62 | 1-1,563 | 124 |

SEQ ID NO: 1-124 are set out in the attached Sequence Listing. The codes for nucleotide sequences used in the attached Sequence Listing, including the symbol “n,” conform to WIPO Standard ST.25 (1998), Appendix 2, Table 1.

While in the foregoing specification this invention has been described in relation to certain preferred embodiments, and many details have been set forth for purposes of illustration, it will be apparent to those skilled in the art that the invention is susceptible to additional embodiments and that certain of the details described herein may be varied considerably without departing from the basic principles of the invention.

We claim:

1. An isolated polynucleotide comprising a sequence selected from the group consisting of: SEQ ID NO: 1-62.

2. An isolated polynucleotide comprising a sequence selected from the group consisting of:
(a) complements of SEQ ID NO: 1-62;
(b) reverse complements of SEQ ID NO: and
(c) reverse sequences of SEQ ID NO: 1-62.

3. An isolated polynucleotide comprising a sequence selected from the group consisting of:
(a) sequences having at least 75%, identity to a sequence of SEQ ID NO: 1-62;
(b) sequences having at least 90% identity to a sequence of SEQ ID NO: 1-62; and
(c) sequences having at least 95% identity to a sequence of SEQ ID NO: 1-62,
wherein the polynucleotide encodes a polypeptide having substantially the same functional properties as a polypeptide encoded by SEQ ID. NO: 1-62.

4. An isolated polynucleotide comprising a sequence selected from the group consisting of:
(a) nucleotide sequences that are 200-mers of a sequence recited in SEQ ID NO: 1-62;
(b) nucleotide sequences that are 100-mers of a sequence recited in SEQ ID NO: 1-62;
(c) nucleotide sequences that are 40-mers of a sequence recited in SEQ ID NO: 1-62; and
(d) nucleotide sequences that are 20-mers of a sequence recited in SEQ ID NO: 1-62;

5. An isolated oligonucleotide probe or primer comprising at least 10 contiguous residues complementary to 10 contiguous residues of a nucleotide sequence recited in any one of claims 1-3.

6. A kit comprising a plurality of oligonucleotide probes or primers of claim 5.

7. A genetic construct comprising a polynucleotide of any one of claims 1-3.

8. A transgenic host cell comprising a genetic construct according to claim 7.

9. A transgenic non-human organism comprising a transgenic host cell of claim 8.

10. The transgenic organism of claim 9, wherein the organism is selected from the group consisting of Lactobacillus species.

11. An isolated polynucleotide comprising a nucleotide sequence that differs from a nucleotide sequence recited in SEQ ID NO: 1-62 as a result of deletions and/or insertions totalling less than 15% of the total sequence length.

12. The isolated polynucleotide of claim 11, wherein the nucleotide sequence differs from a nucleotide sequence recited in SEQ ID NO: 1-62 as a result of substitutions, insertions, and/or deletions totalling less than 10% of the total sequence length.

13. A genetic construct comprising, in the 5′-3′ direction:
(a) a gene promoter sequence; and
(b) a polynucleotide sequence comprising at least one of the following: (1) a polynucleotide coding for at least a functional portion of a polypeptide of SEQ ID NO: 63-124; and (2) a polynucleotide comprising a non-coding region of a polynucleotide of any one of claims 1-3.

14. The genetic construct of claim 13, wherein the polynucleotide is in a sense orientation.

15. The genetic construct of claim 13, wherein the polynucleotide is in an anti-sense orientation.

16. The genetic construct of claim 13, wherein the gene promoter sequence is functional in a prokaryote or eukaryote.

17. A transgenic host cell comprising a construct of claim 13.

18. A transgenic organism comprising a transgenic host cell according to claim 17, or progeny thereof.

19. The transgenic organism of claim 18, wherein the organism is selected from the group consisting of Lactobacillus species.

20. A method for modulating the activity of a polypeptide in an organism, comprising stably incorporating into the genome of the organism a polynucleotide of any one of claims 1-3.

21. The method of claim 20, wherein the organism is a microbe.

22. An isolated polypeptide comprising an amino acid sequence selected from the group consisting of: SEQ ID NO: 63-124.

23. An isolated polypeptide comprising an amino acid sequence selected from the group consisting of:
(a) sequences having at least 75% identity to a sequence of SEQ ID NO: 63-124;
(b) sequences having at least 90% identity to a sequence of SEQ ID NO: 63-124; and
(c) sequences having at least 95% identity to a sequence of SEQ ID NO: 63-124,
wherein the polypeptide has substantially the same functional properties as a polypeptide of SEQ ID NO: 63-124.

24. An isolated polypeptide encoded by a polynucleotide of any one of claims 1-3.

25. An isolated polynucleotide that encodes a polypeptide of any one of claims 22 and 23.

26. A fusion protein comprising at least one polypeptide according to any one of claims 22 and 23.

27. A composition comprising a polypeptide according to any one of claims 22 and 23 and at least one component selected from the group consisting of: physiologically acceptable carriers and immunostimulants.

28. A composition comprising a polynucleotide according to any one of claims 1-3 and at least one component selected from the group consisting of: physiologically acceptable carriers and immunostimulants.

29. A method for treating a disorder in a mammal, comprising administering a composition according to claim 27.

30. A method for treating a disorder in a mammal, comprising administering a composition according to claim 28.

31. A method for modifying a property of a microbe, comprising modulating the polynucleotide content or composition of the microbe by transforming the microbe with a polynucleotide of any one of claims 1-3.

32. The method of claim 31, wherein the microbe is used in the manufacture of a milk-derived product, food product, food additive, nutritional supplement, bioactive substance or probiotic supplement.

33. A method for modifying at least one property of a product, food, food additive, nutritional supplement or probiotic supplement, wherein the product, food, food additive, nutritional supplement or probiotic supplement is prepared from milk, the method comprising adding a polypeptide of any one of claims 22-24 to the milk.

34. The method of claim 33, wherein the property is selected from the group consisting of: flavor; aroma; texture; nutritional benefits; immune system modulation; and health benefits.

35. A food product comprising an isolated polypeptide of any one of claims 22 and 23.

36. The food product of claim 35, wherein the food product is derived from milk.

37. The food product of claim 36, wherein the food product is selected from the group consisting of: cheese; and yoghurt.

38. The food product of claim 35, wherein the food product has at least one modified property selected from the group consisting of: flavor; aroma; texture; nutritional benefits; immune system modulation; and health benefits.


SEQUENCE LISTING (124 sequences)

SEQ ID NO: 1 - DNA, 711 bp, Lactobacillus rhamnosus
atgtggaaagttattaccgatgcagttccgcaaatgatagcagccggcattaaatacaccattccaattgcactcgtgtcatttgcgatcggcctgatcatcgcacttgtaaccgcattgacgcgcatatcggttcgtaaaggtattttgatccgaatcgcaaaaggaatcgccgttttttacgtttggctctttcgctcaacgcctttgctggtgcagttattcatcgttttcttcggcttacccagcctcatcatcccgggtattttcccgcatggcatcaagttagatcccgcggccgcgggaattataacattctcacttaacacgggggcgtattgtgccgaaacgacccgcgcctcgctgttgtcgattgattccgggcaatgggaagcggcttatgcaattggattgccgcggcgactggtgctgcgcgaaatcattattcctcaagcactacgaacggccatcccgccactgtcaaatagtttcatcagcctgatcaaagacacatcccttgctgcctcgatcaccattgtcgaaatgtttcaagtcagccaacaaatcgcggcggaaaattaccaaccattactgatgtactcaatcgttgcgcttctgtatgccattgtctgcactttcttagcttggggtcagcggtatctcgaaaaattcacatcacgctacaatgccaatgcacaaaccacgcaatta

SEQ ID NO: 2 - DNA, 1425 bp, Lactobacillus rhamnosus
ctgatttgcaaaggtcgaagcttgaagccattcggacattttattgatgccatcacggtaaatcgagaacacgtcttgacaaccgctgccgaggccttgattgcaagtgcgggcgatgccttaaatgccagtcacgcgaccttcaatgtattaaacaactctgatctgcaattcgggtttgttgaaaatgaagatggcgaaaccgtccagcttagtaacggtttgtacggtcagttgattcgctcgactaaccgtaaattacgcaaggaggctttcgaggcccttctgcgtgcttacgaaagtttaaaaaatacgtttgcgcaaacgttaagtggtcaagtgaaagcccataatttcaacgcgacagcgcatcattacaaaaatgcgcgtgctgccgctatggcaagtaatcacattccggaaagtgtgtacacgacgcttatcgaccaggttaacacccacctgccgcttttacatcgttatgtcgccttgcgtaaaaaggtgttggcagtcgatcagttacacatgtatgacatttacacaccactgactggccagccgccgttgacttatacgcttgaacaagccaaagcagaggccttaaaagctttagccccactaggcgatgattatcttgagcatgttcgcgaaatttttgacaatcgctatattgatgttgtcgaaaataaaggcaaacgttcaggtgcctattccggtggtgcttatgataccaatccgttcatcttattgaactggcacgatgccgttgatgaactctatacgttggttcacgaaaccggccacagcgtccacagttggtacacgcgtcacaatcagccgtatgtctatggtgattatccgatctttgttgccgaaattgcttcaacaaccaacgaaaacctgttaactgattatttcctgacacattcggatgatcccaaagtacgagcctacattttgaattactatctcgatggttttaaaggaactgttttccgtcagacacaatttgccgagttcgagcactggattcaccaacaggatcagcaaggcgaaccgttgacggccaccagcatgtcacaatattacgccgatctcaacgcccggtattatggaccggaagttgcacgcgatccggaaattgcctttgaatgggcccgcattccgcacttctattacaattactatgtttaccagtatgcaaccggctttgctgctgcctcaaccttagcagcgggcatcagtagcggtgaacctgacgcggctgcccattacttagactatttgaagtcagggagttctaagtatgccattgacaccatgaaaacagctggcgttgatatgaccaaacccgattatcttgaagccgccttttcagtatttgaacagcgcttgacggaattggaaaaaatcttgcagaaagga

SEQ ID NO: 3 - DNA, 1104 bp, Lactobacillus rhamnosus
atgtttaaacccaccattcatcaacttcatccctatacgccagaaaagcctcttgccgtattaaaagaagaacttggcttgccacagctggtgcggatgtcagcaaacgaaaacccattcggtaccagcgtcaaagttcagcaggccgtgaccaactggaattttacgcaaagtcgtgattaccccgatggctatgccagtcaactacgcaccgcggtggcaaaacatttagacgttgccgcagagcagttggtttttggcaatggtctggatgaagtcattgccttaattgcccgcacttttttgagcccgggggatgaagtcattgaaccatggccaacattttccgagtaccgcttgcatgcccaaattgaaggggccaccgtgattgatgtgcccgtcactgaaactggcaattttgatttatctgcaatggcgcaggcgctaaccgcgaaaacgaaactgatttgggtgtgcaacccaaataaccccacgggcacgctgctgtcaattgcgacactgaccgaatggctgcgacagataccaaaagacgtgctggttttaatggatgaggcttatattgagttcactgatgactatccagccacgagcgctatcagcttattatcaaagtttccaaacctcgtcgtgctgcgaacattttcaaaaatctatggactggcgaatttccgggtcggcttcggtgtttttcctaaacaacttgttaactacttgcaaaccgttcggctgccttacaatttaagcagcattgcccaagttagcgcacaggcggccttggctgatcaagattttgtcgcgatgacacgcaagcgagtgcagcaagcgcgcgatagttgggaacgctttttaacccaaactggactgccacacacccggagccaaaccaactttcaattctttcaggccccaaaaatgcaggcatcggctttaaaaaagcgcctgctacaacaaggttttcttgtccgtgatggcttaaaacccggctggctgcgcgtcacgtttggcactgaggtacaaaacacggcggtacagcgcatcattgaaacttttcaggcagaactcactgggccaaatgcgctgaag

SEQ ID NO: 4 - DNA, 1107 bp, Lactobacillus rhamnosus
gtgcatttagcaaaaagaatcctcaacgtcgcaccgtcagcgacattggccttaagtaatcagacgaaagacttaaaggcaaaaggtgccgacgtcattgatttgtctattggccaaccagatttttcaacccctaaggcgattgatgacgcagctattgcggcgattcaggctggtaatgccagtttctatacggcagcaaccggtattccggaattaaagcaggcgattagtgaccggatatttgcccaagacggtattcgttatgatcatcgtcaaatcgttgcaaccaccggcgctaagtttgctttgtatgccttatttcaggttttcttaaacccaggcgatgaggtgctgattcctgttccatactgggtttcctacgaggaacagattaaattggcgagcggcgtgccacatctggtcatgccggcagtcggacataaagtcagtgtcgatgatcttgaggcggctcggaccgataaaacccgggcattgattatcaattcgccacaaaacccaagtggcgttgtctatgatcgcacggaactgaccttaattggcaattgggcgctgaagcatcatattttggtagtgactgacgatatttaccgagatctgatttataacggtacgacttacacctcaatgattagtatcgatcccgatatcgcagcgaatactgttttaatttccggcgtctccaagtcatatgcgatgacgggttggcggattggttatgcggccggtccggaaaagctgattcaggccatggcgacctttattagccacacgacctctaatccggcagcagtttccgaatacgccgcggtggcagctttaactggcgatcagcaggttgttgaaaagatgcgccgtgcttttgaagaacggctgaatcttttctatgatcttctggcagatattcccggtttcgatatgggagataaaccgcaaggcgccttctatcttttcccgaatattaagcgtgccgctcaattgagtcattatggtacggttgatgattttatcagtgcactgttgaccgaaaccggggttgccattgttcctggacgggcgtttgggcatgccggatcatgcgcggat

SEQ ID NO: 5 - DNA, 1170 bp, Lactobacillus rhamnosus
atgacattgcaacctttaaacgaacaactacctgccatcgaggttagtgagattcgacaatttgacgaaagtgtcagtgatattcccggtattttgaaactgacgctaggcgaacctgatttcaacaccccggaacatgttaagcaagccgggatcaaagccattcaggaaaattactcgcattataccgggatggttggtgatccggagttacgcgaagccgcacaacatttttttaaaacgaaatatgccactgactatcgggctacagatgaaattctggtgacagtcggggccactgaagcactggcaaccgccattacgacgatcagtgatccgggtgatgccatgctggttccgtcaccaatttatccgggctacattccgcttctgacgctgaatcacgttacgccgctttatatggatacgagtaaaaccgactttgtcttgacccccgaactcattgaggccaccatcactgcaaatcctgacgctaaaatcaaaggcattatccttaactatccaagtaatcccaccggtgtcacgtatcgggcggcagaagttaaagccattgcggacatcgccgctaaacataacctctacattatctgtgacgaaatttattctgaactgacttatggtgagccgcatgtttccatgggacaatttgcctacgatcgtacatttattgtcaacggtctgtctaaatcacatgcaatgaccggctggcgaatcggctttttgatgggtccccagcagttaatcgcgcaagccaaaaaggtgcaccaatatcttgtgactgccgcaacgaccattgcccagcgcgctggtattgaagctctgacgaacggtgcagacgatgctcaggtgatgaaagcagcttacgttaaacgccgtgattttgtttatgccgccctcatcgacatgggctttagcgtggctcgtcctgatggtgccttttatctttttgcaaaaattccgacccaactgcatctaagctcacgcgaatttacgcacgccttggcacatgaacagaagttagctctgatttcaggtaccgcttttggccccggcggcgaaggttatatccgaatcagttacgcggcatcaatgaccgatcttcaagaagccgttaagcgattgcgcgcgttcatggccagccacatcggc

SEQ ID NO: 6 - DNA, 891 bp, Lactobacillus rhamnosus
gtggcgcgcctcatgcttgatcccggcgatggcctagttgttgaagcgccaacgtatctcggtgccttagccgcctttaatgcttatcagccaacttattatgaaatcccgatgcaagacgatgggatggacattaatgccttacagcgcgttttaatgagtcacaaagtcaagttcatctatacggtacctgattttcaaaatccaaccggcgtcgtaatgtctgtggctaagcgtcaggcgctgattcgactcgccaaccaatatgacgttatgatcctcgaagacaacccctaccgcgatcttcgctatgatggtaaaccgctgccaaccattaagtcatttgacacgcaaggccgcgtcgtttatctcggcagcttcagcaagatcctctcaccaagtctacggatgggctggctcgttgctgcaccggaccttttgcaggaattactagcgttaaaaggcggaagcgacttggaatccagcaacctgaccatgcacggcattgatgcctacatggcggaaaatgacttagacgcccacatcaccgaaatccagaattgttgccgcgaaaagaagaatgccatggtcgcagcgatgaatcgttaccttcctgatgaagcgcacttcaccaaccctgatggcggcttcttcctgtggctcaccatgccagccggcttcgacatgggtgccttcatgaagcaacatctgttaccggaaagcaacatttcctatgtgccttccgccaacctatatgcaacttcggctcaggtcaacggcgcacggctaaacttcaccggtccgacacttgaacagatcgacactggtatcaaagcattaggcgatgcgctcaaaaccgcgctgcagcatcacctagtagccgaacaagct

SEQ ID NO: 7 - DNA, 1170 bp, Lactobacillus rhamnosus
atgaaattgacaatttatgactttgatcatgttatcgatcgccggggtacgtttagcactcaatgggattatattgctgataggtttggccgtaacgatatcctgcccttttcgatctccgatacagattttccagtaccagttgaagtgcaagatgcgctaaaagaacggttaacacatccaatttatggctatacacgatggaatcatgctacttacaaagacagtattgttcactggttcgagcgtgatggtcatacaaagataaacccggattggattgtttatagccctagcgttgtttttacgattgctacactcattcgaatgaagagcgatcccggggacggagtggctgtgtttacgcctatgtatgatgccttctatggtacgattaaacagaacgatcgagtgttgatcccgattcgattagcagctgcagatgaaggctatgtgattgattgggatagtttggcaacggtacttgctgaaaagcagacaaaaatattcttactaacaaatccgcataacccgacaggacatgtttttacaaaatcggaattagcacgcctttatgacttgtgtcaggcagcccatgttttcttgatctctgatgatattcaccgcgatattgtttatccgggtcattcgtacgaaccaatgacaaatgtcggcacaagtgatgttgcactctgctgctcagggtcaaagacatttaacacaccaggcctgattggctcatatgccttcttaccagatcatgatgtaagggcacaatttttgacggaattaaagcagaaaaatgctctgtcttctgtaagcatctttggcatgctggcgcaaattgcggcttataacggttcagaggattacgtggaacaactgactgcctatacaaaaaataatatggagttggttgctagttatttagaggaaaatttgccggaattgcagttttcgttaccggatgccacgtacttagcctggataaatgtgtctaaactgagattaacgtcagaggaacttcaacatcggttagtaaacggcggccatgttggcattatggcgggcaaaacttatggtgataccagatatctaaggatgaatattgcctgtccaaagaagaagttagtgatggggctagaacgtttaaagaagggaattagggga

SEQ ID NO: 8 - DNA, 1158 bp, Lactobacillus rhamnosus
  misc_feature (1)...(1158): n = A,T,C or G
atgatttattttgataatagtgcaacgacgaagatttcgcctgatgcgttggcgacttataacaaggtcagcacggatttttttgggaatcccagcagtttgcatgctctaggaactaaagcaaatgaggttttgcagagttcgcgagcccagattgctaaattaatcggtgctaagccggacgagatttattttacgagcgggggaactgaaagggataactgggtgannttaaagggcactgcatggcttaacgcgaatttggcccgcattctgattacgaccagtatcgagcctccggctgtgatcaatacgatgaaacagctagagaaactgggatttgaagtgacttatttgccggttgatcggcgcggttttattcatattgacgatttgaaagcggctattcgcaaagatacgattttggtgtcgattatggcggttaataatgaaattggcagtatgcagccgattgttcaggccgcgcgggtgttggataattatccgaatattcattttcatgtcgatgctgtacaagccgttggtaaggggttggatgcagcgttgcaggatccgcggattgattttctcagtttttccggccataagtttcatgctccccgcggtaccggctttatctatgccaaagagggtcgcatgcttgatccgttgctaaccggtggcggtcaggaacatgattggcgctcaggcacggaaaatgttccggcgattgcggcgatggccaagtcgctgcgcttacttttggctaatgaagatgctaatgtggcccggcagcaagcagttcgcaagcggatttttgaacatgtcagccaaaagcccaaggtgacgatgtttagtcagttaacaccggattttgccccacatgttttatgttttgccattgctggtgtccgcggcgaaacgattgttcatgcgtttgaggatcatcagatttacatttccacaaccagtgcctgctcgagtaagaaaggcacggaaagcagtaccttggccgccatgcatacggacccgaaaattgctacttccgcgattcgggtgagtttggatgaagctaatactttggatgaagcggatgcgtttaatgcagcgtttgatacgatttatgcaaagtttgccaagcttgataaagcgaccgtc

SEQ ID NO: 9 - DNA, 786 bp, Lactobacillus rhamnosus
atgccaactaaaataggccttcactacaacaaaattggagttgggaaaaccatctactttttacatggcatggggttagacggacacagcatggcagctttttatgagccacgttttaccagcgaagagcggcattttgcccgcctctatccggatttaccgggaatgggaaattcaccggccacgtcagcattgcaatctgctgatgatgtgttggcacaggttcatgctttcattcaggcgaccagcgaagggccatgttatcttgtcggccattcttacggtggctatctagcattaggcctgcttgcacgatttcctgacgagttttccggtgcttttttaaccgctccggttgtactcgcggagaaaacagcacgtacggttgcaacactaaagcatcttattagtgcgccagtgaccagtcagtcaccggaatttaccgactatcaacacatgaatgttgttatcaatccttcaacctggcgacaatatcaggaacttatcctgccagggcttaaaacttttaaccgcgatttctgggttgccatgaagaaccgccatgcttatcgtctgtcgattgaatcacgcttaaccagcctgattaagtcaccagttacgcttgtgttaggtgaaaatgacaatgaagttggctatcaggatcaagtggtctttgcccataaaggcgcacacatgaccacaaccgtaatcccaaacgccggtcataatctgatgatcgatgcgcctgaggctgtcatgaccgcgtttcatcagtttctacacaaa

SEQ ID NO: 10 - DNA, 927 bp, Lactobacillus rhamnosus
atggtaacagcagcagataatattacaggtttaattggcaatacgccgctactcaagctcaatcgcgttgtacctgaaggcgccgcggatgtttatgtcaagctggaattctttaatcccggcggctcagtcaaggaccggattgccttggcgatgattgaagacgctgaatataaaggggtcttgaagccaggcggcaccattgttgagccaacgtccggcaacaccggcattggactggcactggttgcggcggcaaaaggttatcacctcatcatcaccatgccggaaacgatgagtgttgagcggcgtgctttgatgcgtggttacggagccgaactcattttgacgccgggtgccgatggaatgccgggagcaattaaaaaagcagaagcattaagcaaggaaaatggctacttcttgccaatgcaattccagaaccccgccaatccagacgtccacgagcgcacgaccggacaagaaatcatccgttcatttgatggtggcaccccagatgcctttgtagccggcgtcggcacaggcggaacactcaccggggttggtcgggctctgcgtaagatcaatccagatgtacaaatctatgcgttggaagcagcggagtcgccaatgctaaaagaaggccatggcggcaagcacaagattcaagggatctcagccggttttattccagacgtcttagatacgaacctctatcaagacatcattgaagtcaccagcgatcaagctatcgacatggctcgccacgtcagccatgaagaaggcttcctaccaggcatttccgctggcgctaacatttttggcgcgattgaaatcgccaagaaactcggcaaaggcaagagtgtcgccactgtagcaccggataatggtgaacggtatttgtcgacggatttgtttaagtttgatgat

SEQ ID NO: 11 - DNA, 810 bp, Lactobacillus rhamnosus
atgttaaagaaaaagttgtggttcctgttgccgcttgtggccttggtaaccttcacgctcaccgcttgcaccagcgcatcatctgacacgtcaaaaaacagcgacgtcaccgccgaactcatcaacaaaaatgagcttaccatcggccttgaaggtacttatgcgccattttcttatcgcaaagatggcaaacttgaaggcttcgaagtggaactggggaaagccttagccaagaaaatcggggttaaggcaaaattcgtgcccacccaatgggattcgctgattgcaggattaggcagccagaaatttgatctcgtactgaatgatattagtgaaacgcccgcacgcaaaaaggtctacaacttcaccactccgtacatgtactcgcgttatgccttaataacccgcagcgataacaccaccatcaaatcgcttgccgatattaaaggcaaaacatttgtcgaaggcaccggtacacccaatgccgctttagccaaaaaatacggcgctaagatcaccccgtctggcgactttaccgtatcgcttagccttgtgaaagaaaaacgcgcagacggaaccatcaacgcctcggctgcatggtatgcctttgccaagaataactcaaccgcgggcttaaagagtcaaaccctcaaagatagtgtcgttaaacccgatgaagtagctggcatggtcagcaaaaaatcgcctaaactacaagccgcactttcaaagggcattcaagaactacgcaaagacggcacgttgaaaaaactgtcgcaaaaatattttggcaccgatttaaccaccaag

SEQ ID NO: 12 - DNA, 1425 bp, Lactobacillus rhamnosus; identical to SEQ ID NO: 2

SEQ ID NO: 13 - DNA, 768 bp, Lactobacillus rhamnosus
agttatgcgccaaccatcacactagaacaagccaaagaagatattaaaaatgccaccgcactcatgggtcaagattaccaggcacagatgatgcaggccttttcagagcggtggatcgattttcctgctaatcaaggcaaggattccggagcttacactgctggaccgtatggtgtgcacccttatgtcgaaatgacttggagtaatacgctgcctgctgtttacaccttgattcacgaattaggccataccgctcagatggttcgttcacaagaagcccacaatgtgttggatgcggactttaatgcctatttggtcgaaagtccttccaccttcaacgaactgttgctgactcactatctcgaagaaaacgctaaagatccgcgaatgaagcgctttgccttgtcacggttattaaatgatacctatttccacaactttgtcacccatctgctcgaagctgcgtttcaacgggaagtctataacttgatcgataacggcgaaacttttgatgctgctcggttgaacgccattacgcgtaaagtcctgaccgatttttggggatcggcagtagaacttgagccaggtgccgagctaacttggatgcggcaaagccattactacatgggcttatattcgtactcctattcggccggcttgacggttgctacccaagcgtttcaggccatcgaacaacaaggtcaaccagccgttgatcggtggttgcgttatctcagcctaggcgactcacttgatccagtcgaagca

SEQ ID NO: 14 - DNA, 1923 bp, Lactobacillus rhamnosus
ttgttaggtcagtttggtgttgatctcactgaacaggcacgcaaaggtcaaattgatccagtcatcggtcgtgataaggaaatttcacgcgtgattgaaattctgaatcgccggaccaagaataatccagttttaatcggtgaagccggggtcggcaaaaccgcggttgttgagggactggccttaaagattgctaatggcgacgttccagccaagttgcaggatcgccatgtgattcgccttgatgtcgtttcactcgttcaaggcacaggcattcgcggtcaattcgagcagcgcatgcaacaattgattgacgaactgaagcaaaataagaatattatcctatttattgatgaaatccacgaaattgtgggtgccggcaatgctgaaggcgggatggacgccggcaacgttttgaaacccgcattggctcgtggcgaattacaactagtcggcgcaacgaccagcaacgaataccgtcagattgaaaaggattctgccctcgctcggcgccttcagccggtgatggttgaagagcctagcgttgatgaaaccattaaaattctgaaaggtctgcaaccgcgctaccaagacttccaccatgtgaaatacacggaaggcgcaattgaagctgcggcaaccctcagcaatcgttacattcaggatcgtttcctccctgataaagccattgacttgttagacgaagccggttcacgcaagaacctaacgattgccaccgtggatcctgaaaccattaaagctaagattgctgatgccgaaaagcaaaagcaagccgcactcaagcaggaagactatgaaaaagccgccttctatcgtgatcaggtcaccaagttagaagacatggccaaaaagcaatccaacctgccagataatgaaatcccaacagttaccgaaaaagacatggaaaaaattgttgaagaaaagacaaacattccagtcggtgaactcaaagctcaggaacaggcgcaactgaagaatctcgccagtgaccttgagcaacacgtcattggtcaaaatgaagcagttgataaagttgctcgggcaattcggcgcaatcgtatcggcttcaataaaaccgggcggccaattggctcattcctctttgtcggaccaaccggtgtcggcaaaacggaactggcaaaacagctcgctaaagaactattcggttctgaagatgccatgattcggtttgacatgtcggaatacatggagaagttcagcgtctctaagctcatcgggtcaccgccaggctatgtcggctatgaagaagccggccagctaactgaaaaagttcggcgcaatccatacagtttgattttgcttgatgaaattgaaaaagcccacccggatgtcatgaacatgttcctgcagattctggatgacggccgcctaaccgattcacaaggtcgaactgtttccttcaaagatactatcatcatcatgacttctaacgccggatcaactgatgcggaagctaacgtaggctttggtgcaacgttaagcggtaaaacccacagtgtgctggatcagctgggtaactacttcaaaccagaattcctgaatcgctttgatgacattgttgaattcaagccgctttctaaagacgaccttttgaagattgtgtcactgatgattaatgacactaacaacaatctcaagagtcagggattaacgattcacgtcaccgatcccgtcaaagaaaagcttgtcactctgggttacaatccatccatgggggcacggccattgcgtcgggttatccaggaacagattgaagaccgtgtggctgacttttacctcgaccatcctaatgccaaggaacttgaagcaaggatcagcaacggagaaatcacagttggcgaaccagccaaggcagaagcctcttcaaaaacagccaagaaa

SEQ ID NO: 15 - DNA, 1443 bp, Lactobacillus rhamnosus
accaagtcagttgtcggtgtagcgccagaatcacaattattagcgatgaaggtgtttaccaattcagacacatcagcgacgactggttcgtcaacgcttgtttctgcgattgaagattcagccaaactgggcgcggatgttttgaatatgtcccttgggtctgtttccggcaatcaaacactggaagatcctgaaattgcagccgttcaaaacgccaatgaatccgggaccgcagcggttatttcagcaggaaattcgggtacatccgggtcaggtacagaaggggtcaacaaagattattatggcctgcaggataatgagacggttggcacaccggggacatcacgcggggcaacaactgtcgcatcggccgagaatacagatgtcatcaaccaagctgttacgattactgacggcagtgggttaaaactcgggcctgaaaccgtacagctttcaagcaatgactttgttgacagttttgatcagaagaaattctacgttgtcaaagacgcaagcggtaagttaagtacaggtgatgctggcgactatacggcggatgccaaagggaaaattgcgattgtcaaacgtggcagtctgactttcactgacaagcagaaatatgccgaggccgctggtgcagcaggcttaatcattgttaataatgatggcacatccacacctttgacttcgatttctctaacggctacttttcctacttttggtctttccaatacgactggccaaaaactggttgattgggtaactgcgcatccaaatgacagtctgggcgtaaaaattgccttggcattgttgccaaatcaaaattataaagcagatcggatgtcaagtttcacttcatatggccctgtatctgatctttcctttaagcctgatattacagcgccgggtggtaatatttggtcaacgcaaaacaacaatggttatacgaatatgtcggggacctcaatggcatctccatttatcgccggctcccaggcacttctaaaacaagcgctaaataataaagacaatgagttctatgccgattacaagcaacttaaaggcacagcattaaccgattttctcaaaacagttgaaatgaatactgcaaagccgattaatgatattaactatgataatgtcatcgtctctccacgccgacagggagcagggttggtggatgtcaaagccgcgattgatgctttggagaagaatccatcaacggttgtttcggaaaacggctatcctgctgttgaattgaaagatttcacaagcactaccaaaacgtttaagttgacgtttaccaaccgcaccaaacatcagctgacgtatcaaatgactagcaatgaagataccaatgcggtttatacttcggctaccgatctagaatcgtttatacaaagcagtaaaatggctaaactaattcatgaaaggggcgcggca

SEQ ID NO: 16 - DNA, 993 bp, Lactobacillus rhamnosus
atgaccattaattggcagcaagaagttgaaaaattggaaccccaacttctctcagatctaaccacacttttgaagatcaattcggaacgtgatactgatcatcaaaccgatgagtatccactcggaccgggaccggccaaagcgcttgaagcatttttggcgattgcacagcgggacggtttcaaaacattaaatgtcgaccatgttgccggccgcatcgaattaggcgatggcgatgaaatctttgggctttttggccatgttgatgtcgtgcccgcgggaccaggctggcaaaccgatccatttgaccccgttattcgcgatggcaagatttatggccgcggaacaagcgatgacaaaggcccaagtatcgctgcttactatgccttaaagcttattcgcgatctcaagttgccgattaataaaaagattcacttcattcttggtaccgatgaagagtctgactgggtcggtattcaccgctatctcgaaactgaacctgctcccgacttcggattttcaccagacgcggaatttcctatcatcaatggcgaaaaagggattgctagttttgaaatcgttcaaaaaccaatcgccgctgcaaccgctgatctaacgttgaatcatttttccgccggtattcggccaaacatggtgccacaagaagcaaaggctgtcctcagcgggccattaccggaagcatttgtgacgcaagccgagaaatgggcagcggagcaagaagtcaccctcactctgacactaggcaacccgacaacgattgaattgattggaaaaggcgcccatgcccaagaaccaaaagatggcaaaaacgccgcaacctatttagcaacgcttttggccgacttaccatttgatccagccgggaaagcctatctgaccatgattgccaaccaccttcatctggactcacgtggtcaccatttagggattaattataccgataaactaatgggtgacctgaccgcaagtccg

SEQ ID NO: 17 - DNA, 1032 bp, Lactobacillus rhamnosus
ggcaaaatgagtctgtatgctggcgggcctgacgagcggttgacgcctttgattgatggtcggcgtcatgtgacggactttgcattgacaccagatcatcgtggggtggttttcactgagagtacgatgaccattccgagtcggctggtttattttgatttggcatcggaagaagagcaggttttgtatgacccgaatcgtcaggtaacacgtcacttgggcttagttacccctcaaacgtttaattttcaacgagacggttttgagattgagggctggtattttccaccgcaacaggcgtcatcatcgcatccggcaattttgtatgtccatggcggcccagcagtcggatatggctatacctttttccatgaaatgcagtatctggcagcaaaaggctatggcgtgatttgtcgaaatccgcgtggagggttaggttaccgcgaggcatttacgggcgctgtcattaaacattaaccggcaggcgattatgaagattgcttggcttcgggtgaagaagcgctaaagctcgatacaacaattgatccgcaacgtctatttgtcactggcggttcttatggcgggtttatgactaactggattgtgacccatacgcatcgttttaaagcagcggtaacccagcgttcgatttccaattggctgagtatgtatggtaccagtgacatcggttattactttacaccgtgggaactagaaggaaagtggactggcgatttgtcagatgtgcaagggctttgggatttttcaccattagctcacattgatcatgccagaacaccgacgcttgtgatgcacagtgaaaatgatgaacgctgccccatcggcccaagtagaaaagttgatcatcggtctcaaactgcatggtgttgaaaccaagttcatgcgtttcccaaagtcaaatcatgatttgtccccgcagcggggttgccgaatttgcgagtggcacgattgcaggcaattgtggattggtttgacgcccatcaagcacaaccgcagatggctaaaggagaa

SEQ ID NO: 18 - DNA, 1674 bp, Lactobacillus rhamnosus
catttaatcggtgcgacgacgctggacgaatatcgcgaaaatattgaaaaagataaagcattagagcgccgattccaacgggtgctggtacaagagccaactgtggaagatacgatcagtattttacgtggcttgaaggaacggtttgaaatttttcacaaagtgcgcattcatgattcggcgttggtggctgccgcgacattatccaatcgctatatcacggatcggtttttaccggataaggcgattgatttagtcgatgaagcctgtgccacgattaatgttgaaatgaactcgcgcccaactgaactggacgtggccgagcgtaagcagatgcagcttgaaatcgagcagcaggcgttaaagaatgaaagtgatcccgcaagtaagaaacgcctggaaaatgcaaacgccgaattggcaaatttaaaagaaaaaaccaataaactcaaagcacagtgggaagctgaaaagaaggacattcgccagcttaacgagaagaagtcagcgatcgacaaagctaaacacgaactggaagatgcccagagccgttacgatttggaaacggctgctcgtctgcaacacggaacgattccacaacttgaaaaagaattgcaggcaatggagcacagtgatcggccgcagtcttggctggtccaagaaagtgtcacggctaatgagattgctgctgttatttcacgagaaaccggtattcccgtggcaaaactggttgaaggcgatcgtcaaaaattgctgcatcttgccggtaatctgcatcagcgtgtcattggtcaggatgaagccgtcacggcagtttcagatgcggtattgcgttcgcgcgccggactgcaagacccaagccggccattagggagttttcttttccttggtccgaccggggtggggaagaccgaactcgcaaaagcgctggccgaggatctgtttgattctgaaaaacacatggttcgaattgatatgtccgaatatatggagaaggcgagtgtttcacggttggtcggtgcggctccgggttatgtcggttatgaacaaggcggtcaattaaccgaagcagttcgccgcaatccgtatacgattgtcctattagacgaaatcgaaaaagccaatccggatgtcttcaacattcttttgcaagtgttagacgatgggcggttaacggacgggcaaggacgcaccgttgactttaagaacacgatcatcattatgacctctaacctcggctctgaatatttgctggatggcgtgcaaaaagacggaactgtcagtcagcaagctaaggatcaggttcgtcagttgatcggtaaagcttttaaaccggaatttctcaatcgcattgacgatatcatcatgttccatccactttcactagatgatgttaagaagattgccgttaaggatctgcatgagctgggaacacgcttggcagaccagcaaattagtttggacatcacgccagaagctcagacctggttggcggataagggctatgacccggcatttggtgcgcgtccgttgcagcgtctaattaccagtgccgttgaaacgccgttagccaaagagctcattcgcggaacgattcagcccggtcaggaagtggtcataaccgttgcggatgatcagctgcaattcaaagcaaaacaagtagtagcgaaggca

SEQ ID NO: 19 - DNA, 876 bp, Lactobacillus rhamnosus
atttcggcgatcatcgtgattgtagaggagaataatgtggcagcaagagaattaattttagcattcgaaagcagctgcgatgaaaccagcgtggccgttgtcgaaaatgggaccaaaatcctatcgaacatcatcgccacgcaaatcaagagtcatcagcggtttggcggcgttgtaccggaagttgccagtcgtcaccatgtggaacagattacgttggtgaccgatgcggcattaaaagaggcaggtgtgacttatactgacctgaccgcagttgccgtgacgtatggaccgggactggtaggtgccttgttgatcggggtacgggctgccaaacccattgcgtatgcccatcacttaccacttattccggtcaatcatatggcaggccatatttatgcagctagatttgttaagccattggtctatccattgttggcattagcggtttccggcgggcacacggaactggtctatatgcgcgctgccggtgaatttgaaatcatcggtgatacccgtgacgatgcggccggtgaagcgtatgacaaagtcggccggatattgggtatcccttacccagccggaaaagaagtcgatagattggcgcatcttggtcatgatacctttcattttccgcgagccatggataaagaagacaatctcgattttagtttcagcggtttgaagtcagctgtcattaatacggtacatcatgccgatcaaattggggaatcactcagccgtgaagatctgtctgcgagttctcaagcgtcagtggtgcacgtgatggttctcaaatcccaatcagcgatagccgaatatccggttatacaggtggtgatcgccgggggcgttgccgataatcaagggctgaaa

SEQ ID NO: 20 - DNA, 732 bp, Lactobacillus rhamnosus
atgattttccgcaaaccacaaccattcgaatatgaaggtaccgatactggcgtggtattgttacatgcatacacgggtagccccaatgatatgaattttatggcgcgggccttgcagcgatccggttatggggtttatgttccgcttttttccgggcatgggacagtggagccgttagatattttgacaaaaggcaacccggatatttggtgggcagaaagtagtgccgcggttgcgcatatgaccgcaaaatacgccaaggtgtttgtttttggcttatcactgggaggtatttttgcgatgaaggcgctagaaaccttgccagggattacagcaggcggtgttttttcatccccgattttgccgggcaaacatcacttagtaccgggttttttaaagtatgccgagtatatgaatcggttagcaggcaaatcagatgaaagcacacagattctggcatatttgccgggacagttggccgcaatcgatcagtttgccacgacggttgctgctgatttaaatttagtcaaacagccgacttttattggacaagccggtcaggatgaattagttgatggtcgattagcgtatcaattacgcgatgccttaatcaatgctgcacgcgttgattttcattggtatgatgatgccaagcatgtcattaccgttaactcggcccatcacgcattagaagaagacgtaatcgcatttatgcaacaagaaaacgaggga

SEQ ID NO: 21 - DNA, 1299 bp, Lactobacillus rhamnosus
ctgggtatatttttctttaaacgcttcaggaagttgcacttgttcgatcctttaaattatccggaagagacttttcaaagtttcgacagcgcttttaacaacggagctgattacgttgagcttgacgtacatgaaagtgcagatggtgtgattgtgattcaacatgacaccacgattcagcgaacgactggtgccaacttggcgatcgcgaaaacaaacttcgcacaacttcagcaatatcataccaaaaatggcgaaccgattcacagcctagaggaactcttcgcccatgagcaacaaacaaagcataaattcctgattgaaactaaaattgtaaaaggtgaaccgcatccgcatctagaagacaaagttgcggccctgatcaagcaatatcacatggaaaatcgcgtgatgtttcattcattctcagcagctagcctcaaacgcttgcaagcagctctgccaaatattccgcgaattttgatcgttggctcgcttaagcggatcaactttgacgtcttgacgtacgtggacggtattaatctaagttccgatttagtgacgccgcaacttgtcacccaactgcatgatctaggtaagaaagtttatgtttgggatgaaatgaacgaggatcgggcgaaatggacttggctcgtcaatctcaacattgatggcgtcgtcactaattacaccagcctcggccacgaatttcaaacgcttaaggcagctgctgtcaccaccagcatcaatgatcttggcgcaaactcaagccttgctgcactgccagtttatgaaaatccttatcagccattgttgcgctctgaacggctggcaccgcaaaccccgatcatgatttccagcatggtttccctcgctggcagcacgtactaccaaattggcgataatgcatttgtccctgccgaaaccattaaccttgcccctgaagccggttgggcaagtctttttctccatcagcgcatcgtcatcaccagccggcactttaaggtacccgtgcacgctgatcccttacatcagcaagccattaccggccatgttggcaatcacaaatgctaccgggtattagcagcccgttatcaaagcggccagctgtatttaaaaacaaaaatcggttggttaaacgccaaagatttacaggtgctgccaaccgccgagaatatgcgcatctggctcacgctctatcgcagcatccccgaaaaccaaaaaccgctccttcactgggcacttggcgacacggccttcgatacaccgcttctcaatgccagtgtcctgaacatcggt

SEQ ID NO: 22 - DNA, 1344 bp, Lactobacillus rhamnosus
atggaattagcgaaactcgctgttgacgaaaccggccgtggtgtttgggaagataaagccattaaaaatatgtttgccactgaagaaatctggcattcgattaagaacaacaagaccgttggcgttatcaatgaagataaacaacgcggcttggtatccatcgccgaaccaatcggggttattgccggggtaacgccggtgaccaacccgacatcaaccacgatctttaaatccgaaatttccatcaagacccgtaatccgattatctttgctttccacccgggtgcacaaaagtcttcagcgcgtgcgttggaggtcattcgggaggaagctgaaaaggccggattgccaaaaggggccttgcagtatattccggttccaagcatggaagcaactaagacactgatggatcatcccggcattgccacgatcttggcaaccggtggccctggcatggttaagtcagcttattcatccggcaaaccggccttgggtgttggcgcagggaatgcaccggcatacatcgaagcaagtgccaatattaagcaagccgttaatgatttggtcttgtccaagagttttgataacggtatgatctgtgcttccgaacaaggggccatcgttgattccagcatttacgatgccgcgaagaaagaatttgaagcccaaggtgcctattttgtcaaacctaaggacatgaagaagttcgagagcacggttattaaccttgagaagcaaagtgttaatcctcgaattgttggccaaagtcctaagcaaattgctgaatgggcagggattcgaattcctgatgacacgaccatcctgattgccgaactaaaagacgttggcaagaaatatccgctttctcgggaaaaactgagcccggttttggcgatggttaaagccgatggtcatgaagatgccttcaagaaatgtgaaaccatgttggatatcggcggcttgggacacaccgcggtgattcacacagctgacgacgaattggcattgaaatttgctgataccatgcaggcttgccgaatcctgatcaatacaccttcttctgttggcggtatcggggatctctacaacgaaatgattcctagtttgacgctgggctgcggctcctatggcggcaactcgatttcgcacaatgtggggacggttgacttgttgaatatcaagaccatggcaaaacggcgcaacaacatgcaatggatgaaattgccgccaaagatttatttcgaaaaaaactcggttcgctatctggaacacatggaaagcatcaagcgcgccttcatcgttgctgatcgttcaatggaaaaagctgggtttcgtcaagatcat

SEQ ID NO: 23 - DNA, 474 bp, Lactobacillus rhamnosus
gtgttggtcaataatgccggcattaccgacgatatgttggcgatgcgcatgaaacccgcttcttttgccaaggtcgttcaggttaacttggatggcaccttttatgttacccaaccggccttcaagaaaatgctaaaggcccgcgctggcgtcatcatcaatctggccagtgtggtcggtttgaccggtaatatcggccaagccaattatgcggcaagtaaagcaggcatcatcgggctgactaagacgctagctcgtgaaggggctatgcgtggcgtgcgcgttaatgccattgccccagggatgatcgccaccgatatgaccgctgccttgagccaatccagtcaggaccagattctggcggaaattccgttgaagcggttcggtcaacctgaagaaattgcccacacggcccgttttctggtcgaaaatgcctacataaccggtcagacagtgactgtcgccggcggatta

SEQ ID NO: 24 - DNA, 1002 bp, Lactobacillus rhamnosus
atgtatcatgcagcagctgatcgttatgagaaaatgccggttcgccatgctggtaagacagggttgatgttgccggttatttcgttgggattgtggcagcattatggcaacttggatccatttggcccgcgacgctcggtgattttggatgcgtttgatcgtggcgtttttcattttgatgtcgctaatcattatggtaatggtgatcgtgaaccgggatttggctctagtgaaaggttactcgggcagattctggccacggatttaaaaccgtatcgagacgaattggtgattagtaccaaggtgggttatgagattcaccctggtccatacggtgtcgggacgtcgcgtaaagcagttattcaaggcttgaatgattcactcaagcgcttgcagttggattatgtcgatatttactatgcccaccgatttgacgataccgtggccttggaagagacggttaatgcgctggatcaaacggtgcgtgacggtaaggcgttgtatattggtatttccaactatgatacgaagcagaccaaagaagcaattgcgatgtttaaagatctgcacacgccttttgtactgaatcaatacagttacaacatgtttaatcgcaccgctgaaacgtccggcttgatcgatgcattaaaagctgatggtgccgggttgattgcatacggaccgttatcagaaggcttgttatcagatcgctacctaaagggaattccggatactttcaaaatccatccaaccaacaaggccacttttgctaagggcaaagaggctgtggttaagcaactaaatgcgcttaatgaaattgcgcatgatcgtgaccaaaccctgagtcaaatggccttggcgtggttgttacgggatccggttgtcacaagtgtgatcattgggacgacctcagttgaacaccttcaggataaccttaaagcaacggaacatctgacctttactgctgaagagattcaacaaattgatgatattttaaatgct

SEQ ID NO: 25 - DNA, 1239 bp, Lactobacillus rhamnosus
gccgttgcattaccgttacttggcgttttggctatcgccgcaacgcatgctgaaggtgtttatgatattggccgtccacttggccgcttcttcgccttagcgttcatggtgctcattcatgccacgatcgggccaatgttcggcacgccgcggactgccaccgtttccttcaccaccggcgtcttaccgatgttaccaaaagcctggcaacaaggcggcttgcttgtcttttcggctttattctttggggctgcttttttcctgtcatacaaggaacgcaaaattaccacagctgttggtaaagtcttaaatcccgtcttcttgctattgttattcttcgtcttcttcattggtttcttacacccaatggggaatccggctgctcaaacagtaacggctgcgtacaaaaatggcggcagtttcatgagcggcttcctgcaaggttacaataccatggacgcgcttgctgccttagcctttggtgtgactgtcgtaacggcggttcgcggtttaggcttgaaaaacgatgatcatgtcgccaaagcaacggctaaagctggggtcatggctacgagttggatcgcgttaatctacgttgccttgatcgtcttaggaagtatgtccctggcccactttaagcttagtgctgaagggggaactgcttttaatcaagtgggtacgttctactttggtactgttggccaccctgcttggcaaccttgcttaaccctgacctgtttgaacaccccggttggttttgtcagggcattcccgcacgacttccaccggcatttccctaaagtcagctatcaggtctggcttggattgacaagttttctgtcattcttaaccgccaacttcggacttgaacaaatcattgcatggtccgttccaatgctgatgttcctatatcctttctcaatggttctcatcttactatcggtctttggcaaagcgttccatcacgatccactagtttaccgaatcgttgtagcattcaccatcgttccggctgtgctcgacatgtttgcagctttccccgccgttgttagtcaaagttcgttaggcttggcattgcatagcttccagcttcattttctaccattttccgcaatgggtctcggctggctcgtgccggctggtgtgggtctcgtccttggcctcgtcgcacatgccgtcaaagttcgcaaagcagtcgcagcaactcatctcgaagctgaacaaactcagctagtacac

SEQ ID NO: 26 - DNA, 1881 bp, Lactobacillus rhamnosus
atggcagacaatcacaaagcccaaacgaccaagcagccatctggcccacggatgggtcctggccgtggtggtctcgttgaaaagccgaagaacttctggggcacaacagcgcggttatttggttacatgcgcaaccgtctgattggtattattgcggtgctagtcttggccattgcttcgaccgtctttcaaattcgcacgccaaaaatcctaggggaagctacaaccgagatttttaaaggggttatgaaaggccaagcggagcaaaaggccggtatcgctgttggcaactatccaattgattttgataagatcaaacagattattttaatcgtcttggtgctgtatttgggtagcgcactgtttagtttcttgcagcagttcatcatgactcggatctcgcagaatacggtttaccagttgcgtaaagatctcaagcacaagatgaagactgtgccgatcaagtattatgacacgcatagtaatggcgatattatgtcacgggcgattaacgatatggataacatcgcctcgacactgcaacaaagtctgacgcagatggtgaccagtgcggttatgttcgtgggaaccatttggatgatgcttacgatttcctggaagttaacgctgattgcgctggtaacgattccgctgggcttaattgttgtcgggattgtcgcgccaaaatcgcaacggttctttgccgcccagcaaaaagctttaggtctcttgaataatcaagttgaagaaacttatggcggtcaggtgatcatcaagagctttaatcgtgaagatgatgaagttgaggcatttgaaggccagaaccaggcattttatgatgcagcgtggaaagcgcagtttgtttccgggatcatcatgccgcttatgattttcctaaacaacattggttacgtgtttgttgcgattatgggtggcattgaagtttccaatggcacgatcacccttgggaatgttcaggcgttcctccagtatatgcagcaattttcccagccgatttcccagcttgctaacctagccaatacgattcaatccactattgccagtgccgagcggatctttgcggtgttggacgaagaagatatgcaggatgagccgtctggtgtgccggcagtggccaatgatcctaacaaactggtcatggatcatgttcagtttggttataccccggatgccttgttgctcaaggactataacctgcaagtcaaaccgggtgagatggtcgcgattgtcgggccgaccggtgcagggaaaacaacgatcatcaacctgctagagcgtttttatgatattagcggcggatcgattcgcttgaatggtaccgatacccgcgatatgaagcgagaagatgttcgcgcgcattttgcgatggtgcttcaggatacttggctgtttaccggcacgatttgggataacttgaaatatggccgcgaagacgcaactgacgacgaagtattggccgcagccaaagcagcccatgttgacaactttgtgcggcagttgccggatggctacaacacgattctgaacgaagaagcctcgaatatttcgcaaggtcagcgacagttgttgacgatcgctcgggccttcgtggcagatccggaaattcttattctggacgaggccaccagctcggttgatacgcggacggaaattcatattcaacacgccatgaaccgtttgctgaccgatcgtacgagcttcgtagtcgcccaccggctgtcaacgattcgtgatgccgacaagattatcgtgatgaatcacggctccattgttgaaaccgggaatcatgacgaactaatggctaaaaacggcttttatgctgatctgtacaacagtcagttcagtggcaatgtcgcgatt

SEQ ID NO: 27 - DNA, 606 bp, Lactobacillus rhamnosus
accaccaggctgtcaagtttgatcaccgaatacttagacagccaactagctgaacgtcgcagcatgcatggcgtcttggttgatatttacggtctgggcgtgctcattactggcgattccggggtgggaaaatccgaaactgccttggaattggttcagcgtggtcatcggcttattgctgatgatcgggtggatgtttatcagcaagatgaacaaacggttgtcggagctgcaccgccgattttatcccacttgttggagattcgcggattgggtatcattgatgtcatgaatctctttggtgccggggcggttcgtgaagacaccaccatttcgctgattgtgcacttggagaattggacaccagacaaaaccttcgatcgcttgggctctggcgaacagacgcaaatgatctttgacgtgccggttcccaaaattacgattccggtcaaggtcggtcgtaacttagccattatcatcgaagtggccgcgatgaatttccgcgccaaatcgatgggctatgatgccactaaaacatttgaaaagaatttaaatcatctgatcgaacataacgaagcgaacgaccagaagagttcggaggaaaaa

SEQ ID NO: 28 - DNA, 1023 bp, Lactobacillus rhamnosus
  misc_feature (1)...(1023): n = A,T,C or G
atgtctatctccacgcgtgcaaataaactcgacggcgtcgagcaggcatntgtggcgatggcgaccgaaatgaataaaggcgtgctgaagaatttaggactgctgacgccggagctggagcaggcgaaaaacggcgacctgatgattgtcatcaatggtaaatcgggtgcggacaacgagcagttactggtggagattgaagaactgttcaacaccaaagcgcaaagcggctcgcacgaggcgcgttacgccactattggcagcgccaaaaagcatatcccggaaagtaacctggcggtgatttcggtcaacggtctgtttgccgctcgcgaagcgcgtcaggcgctgcaaaacgatctcaacgtgatgctgttttccgataacgtctcagttgaagatgaactggcgctcaagcaactggcccacgaaaaagggctgctgatgatggggccagactgtggcacggcgattatcaacggcgcggcgctctgttttggtaacgccgtgcgtcgcggcaacatcggtattgttggcgcatccggcaccggcagtcaggagttgagcgtccgcattcatgaatttggcggcggcgtttcgcaactgattggcaccggcgggcgcgacctgagcgagaaaatcggcggcctgatgatgctcgacgccatcgggatgctggaaaacgatccgcaaactgaaatcattgcgcttatctccaaaccgcctgcgcctgcggtggcccgcaaagtgctggaacgtgcgcgcgcctgccgcaagccggtggtcgtctgcttcctcgatcgtggcgaaacgccagtggatgagcaggggctacagtttgcccgcggcaccaaagaggcagcgctaaaagcggtgatgctctccggcgtgaaacaggaaaatctcgacctgcatacgcttaaccagccgttgattgcggatgtgcgtgcgcgtctgcaaccgcagcagaaatacattcgtggcctttctgcggcggcacgctgtgcgacgaaaccatgttcgcgg

SEQ ID NO: 29 - DNA, 1227 bp, Lactobacillus rhamnosus
cagatcctgaataacccatttttaaataaagggactgcttttacgcaggaggaacggaaccaatatggcttgaatggtttgctgccaccagctgtacaaacacttgatcagcaggttaagcaagcttatgcccagttgcagaccaagccaactgatttggctaagcgtcaatttttgatgaccttgttcaatgagaatcatgttttgttctataagcttttctccgagcatatcaacgaattcatgccaattgtttacgatccgactattgccgacacgattgaaaactacagtgcgctttttgttaatccacaaaatgccacgtatctttcaatcgatgatccggaccatatcgaaagcgcactgaagcatagcgcagatggccgcgatattcggttgctggttgtaagcgatgctgaaggcattcttggcattggcgattggggcacacaaggtgttgacatctcagtcggtaaattaatggtttatacggctgctgccggcattgatccgagccaggtcttgccagtggtcttggatgtcgggactaacaatgaaggtttgttgaacgacgacctttatttaggcaatcgtcacaagcgcgtatacggtgaaaagtatcaccactttgtcgataaatttgtcgccgcagcagaaaagctgttcccgaacctgtatttgcattttgaagactttggacgcagtaatgctgcagatattttgaatcaatataaagacaagatcactactttcaatgatgacattcaaggcaccgggatcattgtcttggctgggctattaggcgccatgaatatttccaagcaaaaattgaccgaccaagtttatttgagctttggtgccggaactgccggtgctggcattgcttcgcgagtttacgaggcctttgttgaagaaggattgagcccggaagaagccaagaagcatttctacttggtggacaaacaaggcttgctctttgatgacatgacggatttgacgccagaacaaaagccgtttgcccgttctcgcagcgagtttgctaatgcagacgagctgacaacgcttgaagctgtcgttaaggcagttcatccaacagtcttggttgggacgtcaaccgttccgggtacgtttacagagagtatcgtcaaggaaatggctgcccacaccgatcgaccgattatcttcccattgtccaatccgacgaagctggctgaagctaaagcagat

SEQ ID NO: 30 - DNA, 1158 bp, Lactobacillus rhamnosus
  misc_feature (1)...(1158): n = A,T,C or G
atgatcaaacccgaaaagacaatcaatggaaccaaatggattgaaacgattcaaatcaatgccgaagaacgggcaaccctcgaagatcagtatggcgtcgatgaagatattattgagtacgtcactgataatgatgaaagtactaattatgtttatgatatcaatgaggacgaccaattattcatctttctggcgccgtatgccctcgacaaagatgcgctgagatacattacccagccatttggcatgttgctccataagggcgttttattcacgtttaatcaaagccacatacctgaagtcaacacggcactttactcggcattggataatcccgaggttaagagcgtcgatgcatttattctggaaacactgtttacagttgttgacagctttatcccaatttctcgcggcattaccaagaaacgcaactatttggataaaatgttgaaccggaagacgaagaacagtgacttggtttcactttcatatcttcaacagacgttgacctttttgtccagcgcggtccaaaccaatctcagtgaactcgatctcaacggcagtgacgcccttcagcagattatcgaattgctcaatcagcatcccctcgactntgcgccagatgaaaaaggtgcctattccaatagtaactactatctcctaggacacattattacgcaggttgcgaatatgccgctgagtgattttctcaaccaacacttctttgaaccattggcaatgacgaaaactcaactgggtacgcaacatgctgatgccaatagttacgatgatttggactttactaacggcaaaccagtcgcccttggccgcggccactaccaaggtggagatggcgcggtggtgagttcactcgcagacttggccatctgggcgcgagccgttttacagcgccgcattttgccggaatccgcgtgggatgaggcactgacgctgacccacgacttttacggcatgggttggatgaaatcccgaacacagcactggttaagtcacaatggtcatattttcggttactgggcgttttttgatgtttcatttgaaaagcaattagcacagattacgctgaccaacatgtcgcctggtgttgagacactcaaaaaatggcaagaggagatggctaactggcgcgcatcgtta

SEQ ID NO: 31 - DNA, 1071 bp, Lactobacillus rhamnosus
ttggacaatcaagatgccgattttaagcccacaatccaaattctggatgaagttggcaaagtcgtcaatcctgatattatgcccgatttgagtgatgatcagctggtcgatttgatgtcaaaaatggtttggcaacgcgtgctggatcaaagggcgacagcgttgaatcggcagggacgattaggcttttatgccccaagtgcaggcgaagaggccagcatgatcggtagtcacgctgctatgaagtcatcagattggctgcttccagcttaccgtgatttaccgcaattgattcaacacgggttaccgcttgacaaagcttttctctggtcgcgcggtcacgttgccggcaatgagtatccggaagattttcacgcattaccgccgcaaatcattattggtgcgcagtatgttcaaactgcgggtgttgcgctcggtttgaagaaaaatggcagtgatgaggtggccttcacctatacgggtgatggcggtacttcacaaggtgacttttatgaaggcgttaactttgctgggcatttcaaagcgccggcactgtttattgttcaggacaacggctttgctatttccgtgccgcgggcgagtcaaacggcagccaaaacgcttgcgcaaaaggcggttgcagccggtgttcccggcgtgcaggttgacgggatggacgctttggcagtctatgaagtcaccaaggaagcgcgtgcatgggcggctgctggcaatggaccggttttaatcgagacgttgacatatcggtatggcccacacacgctatcaggtgatgacccaactcgttatcgctccaaagagaccgatgagttatggcaaaaacgagatccgttaattcgaatgcgcaactatttgaccgataaaggcttgtggagcaaagacaaagaagatgccttgattgaaaaggtcaaagatgaaattaaagatgctatcaataaggccgataaagcgccgcagcagacggtatcgcgcttcttgaaagacacctatgaagttgccccgcaaaatgttgctgaacaattggcagaatttcaaggaaaggagtcgaag

SEQ ID NO: 32 - DNA, 1310 bp, Lactobacillus rhamnosus
tctgtactgaacatcaatggcgggaatttgaccctgactgatgatggcgtatctgccggtactctgactggaggtggcttcctgaatatcagcggcggcgtcctcgatattacaggaggcaaccacacttttgctgtcagcaccataatagcaaaagatgcaactgtccgaatgaacgatgtttccggactgggtactggtaacatcagtaacgctgggacattatctcttactcatgcctcaggcttgctaagtaataatctgagtggttctggtacagtatctttgatcaatagtgatacccagatttcaggaaataacagtaactactcagggctgtttgtagtagataccagctctcagttgactgccactggagcgcagaatcttgggattgcttccgtgagtaaccgtggaatcttgcagctgaacaacacaacagactggcaacttataaataatgttactggaaccggtaatgtccgtaaaaccggttccggttcactgactgtccgaagcaatgctgcctggagcggacagacagatattgatgacggctctctgattcttgggcaatctgatgcacctgtcatgctcgccagtagcctggtcaatattgcaaagaacggtaaacttaccggatttggtggcgtagtagggaacgtaaccaatagtggaagccttgacctgcgatcggcggctccgggaaatattctgacgattggaggtaactacaccggtaataatggcacgctgctcattaacacagtgctggatgatagctcttctgcaaccgacaaactggtaattaaaggcgatgcgtccggtaagacccgagtggctgttacgaatgttggtggttcaggcgctaatacgctgaatagtattgaagttattcatgttgatggtaatgcagctaacgctgaatttattcaggccggacgtatagcggccggcgcttatgactacactcttggacgtgggccgggaagcaactatggaaactggtatctgagcagtagtaaaaatactccagaaccaaggcctgatcctgaacccactccggaagggcatgataacaacctgcgcccggaagccagctcctataccgcgaatatagctgcggcaaacaccatgtttgtgacccgccttcatgaacgtctggggcagacgcaatacgtcgatgcaattaccggagaaccgaaagcaaccagtatgtggatgcgccatgaaggaggacataaccgctggcgcgacggttctggtcaactgaaaactcaaagtaatcgttatgtgattcaactgggtgg

SEQ ID NO: 33 - DNA, 645 bp, Lactobacillus rhamnosus
atgaagattttgattaccggcgcacaaggccaactaggcaccgaactacgccacttattggatgcacgcggcattacttatcgggcaactgatgccaaagacttagatattaccgatgaagccgccgttaatcagtactttgcggactatcagccagacgtggtgtatcactgtgctgcctatacagccgttgataaagccgaagacgaagcaaaagcgctcaatcaattggtgaacgttgacggtacgcgtaacttggctaaagcagcggccaaagttgatgcaaccttggtttacatcagcaccgattacgtgtttgatggcgatagtaaggagatttacaccgttgacgatcagccggcgccacgcaatgaatatgggcgggctaaatacgaaggcgaacagcaggtgcaaaagtaccttaagaagtactacatcattcggacttcttgggtctttggtgaatatggtcacaactttgtctacacgatgttgaacctcgccaaaacgcataaggaactgaccgtggtggacgatcatcaagaatctttttccgtctcatcatcacggacatttgtgaaatatcaacacgaacacctgatttattcccgacctgtgccatatcggccccaccttcctggcata

SEQ ID NO: 34 - DNA, 1920 bp, Lactobacillus rhamnosus
  misc_feature (1)...(1920): n = A,T,C or G
atgcttggaggaaaacagatgcctgaagtaaagaaatttgaagccggcacttatgatgtcatcgttgtcggtgctggtcacgctggnnttgtgaagccggccttggctgcagcacgcatgggcgaaaagacattattactgacgatcagcctggaaatgttggcatttatgccatgtaatcccagcttaggcggtccggccaaaggaattgtggtccgtgaaattgatgccctcggcggtgaaatggggaagaatattgatcggacctacatccagatgcgcatgctcaacacgggtaaaggtccggcagtgcgcgcattacgtgcccaagcagataaagcggcctatcaccgcagtatgaaacacgtcattgaagatacgccgcatttggacttacggcagggactcgctaccgaagtgctggtcgaagatggtaaggcagtcggcatcgtggctgctaccggggccatttatcgcgccaagagtattgtgctaacagctggcaccagttcccgcggtaaaattattatcggcgaactcatgtatagttccggccctaacaatagtctgccaagcattaagctctctgaaaatctggagcagctgggctttaagctgcgccgcttcaaaaccggaactccgccgcgtgttaatggcaatacgattgacttttccaaaaccgaagaacaaccaggcgataaaacaccgaatcattttagttttacaacgccggattcggtttatctcaaagatcagttgagttgttggatgacgtacaccaacgcgacgactcatcagattattcgggagaatctggatcgcgcgccgatgttttccggcgtgattaagggagtcgggccgcgttactgtccatccattgaagataaaattgttcgctttgctgataagccgcggcatcaattattcttggaacctgaaggccgcgacacctcggaatattatgtgggggacttttcaacgtccatgcctgaggaaatccagttgaaaatgctgcacagtgtcgcgggattggaacatgccgaactcatgcgcgccggttatgccatcgagtatgacgtcatcgagccatggcagttgaaagcaacgttggaaactaaggttgtggagaatttgtataccgccggacaaatgaacggtaccagtggttatgaagaagctgccggtcagggaattgtggccgggattaatgctgcccgccgcgctcaaggcaaaggacccttcacgttgaagcgttcagatgcttatattggcgtgatgatagatgatctcgtgacaaaaggaacgaatgaaccgtatcgtttgttaaccagccgcgccgagtatcggttgttactgcgtcatgacaatgcggatctgcgcttaacgccaatgggacatgaacttggcctgattagtgatcaacgctatgctgtctttttggctaagcgtcaagccattaccgatgaattagcgcgccttgagcacacccgcctgaagcccaaggatgtcaacccgtggcttgaagctcatcattttgcctcccttaaagatggggtcttagccagtgacttcttgaagcgtccggaaatcaattatcagacgctagaacagttcttaccggaaaacccaaccttggatcatcgggtgattgaacaggttgagatccaaatcaaatacgccggctacattgccaaagaagaagnccagtgtgccaagttgaagcggcttgaaggcaaaaagattccggcacgtatcaactacgaagcaatcaatggcttagcaaccgaggcacggcaaaagctggttaagattcaaccggaaaccattgcccaagcaagccgaatcagtggcgttaatccggctgatgtcgctattttgtccgtgtatattgaacagggacgaattagtaaggtggcacag

SEQ ID NO: 35 - DNA, 762 bp, Lactobacillus rhamnosus
cccctgtcaaccatgatgttggctgggattcgcgatatcttggtcatttcaacgccgagagatattgatcgttttcaggatctgttaaaagacggtaaacaactgggactcaatattagttacaaaatacaggaaaagccaaatggcctggcggaagctttcattgtcggggctgacttcattggcgatgattctgtgtgcttgattctcggcgacaatatcttttatggcagcggcttgtccaagctggtgcagcgctcggcggctaaaacaaccggggcaacggtgttcggctatcaagtcaatgaccctgagcgttttggcgtagtagcctttgacgagcagcatcatgtgcaatcgattgtcgaaaagccagagcatccggagagtaactttgcggttaccggcatgtatttctatgacaaccaagtggtggacattgctaagaaccttaaaccgtcaccacgaggcgaactagagattacggatgtgaacaaagcgtatctcgaacgtggccaacttgatgttgagctgttgggtcgaggatttgcttggttagatactggcacccatgaatccttacatgaggcggctagttttattgagaccgttcagaagcggcagaatcttaaaattgcctgtcttgaagaagtagcctaccggatgggttacattgatcgcgatcaattacgcaaactggcgcagccgcttaagaagaatgattacggtcagtacattttgcgcttagcagacgaagaagac

SEQ ID NO: 36 - DNA, 936 bp, Lactobacillus rhamnosus
atggcaattaacctagttgggattaatgacgcgaatttaacgttaattgaagaaggcctgaacgtccggatttcgccgtttggggacgaattacgcatcagcggcgaaaccgaagcggtcagcttgacactacagctgcttgaggcggcaactaagttattagcacaaggcatcaaactgtcacctcaggatattgctagtgcggttgcaatggcaaaacggggtacactcgaatattttgcggatatgtatagtgagaccttgttacgcgacgccaaggggcaaccgattcggattaagaattttggccagcgtcaatatgttgatgccatcaaacacaacgatattacttttggcattggcccagccggtaccgggaaaacttttctcgccgtggtgatggcagttgcagccatgaaggccggccaagtcgagcggattattttgacgcgtccggccgtggaagcaggcgaaagtcttggctttctccctggtgatctcaaggaaaaggttgatccttatttgcgtccggtttatgacgctttatatgccgttttggggaaagaacacaccgatcgcctaatggatcgcggcgtcatcgaaattgcgccattagcgtatatgcgtggtcgtacgttggacaatgcgtttgcgattttggatgaagcccagaatacgactcaggcccagatgaaaatgtttctgacgcgcctgggctttggctcgaaaatgattgtcaatggtgatgtgacgcaaattgacttgccgcataatgccaaaagtggcttattgcaagcggaacagttattaaaagggattagtcatattgccttcacgcaattttccgcacaggatgttgtgcgccacccagttgtcgccaagattatcgaagcttatggcaaacatgatttacagctgcaaaagcaaacgaaggag

SEQ ID NO: 37 - DNA, 840 bp, Lactobacillus rhamnosus
atgaagaagttcgacaagatgatggactggttagcagatgtctatgtcaatgccctgaatgttattcactacatgcacgacaagtactactatgaagctgcgcagttggcattgaaggatacacgtctgaatcggacgtttgctaccgggatttccggtttgtcacatgcggttgattccatcagtgccatcaagtatggccacgtgaaggcaattcgggatgaaaatggcgttgcaatcgactttgttgctgacaatgatgactatccgcgttatggtaacaatgatgaccgggcagacaacattgctaagtggttggttaagaccttctacaacaagatgaatacacatcatctgtatcgcggtgctaagctcagtaccagtgttctgaccattacatcaaatgtggtttatggtaagaataccgggacaacaccaaacggccgtcagaagggcgaaccattctcacctggagccaacccagcgtatggcgctgaaaagaacggtgctttagcttccttgatgtcaaccgccaagatcccatatcactatgcaacagacgggatcagcaatacctttggggtaacaccgaacaccttaggccatgacgatgaaactcgtaaggacaccttggttcacatggttgacggctacatggaaaatagcggcatgcaccttaacatcaacgtcttcaataaagaaacgttgattgatgcccagaagcacccagaagaatatccaactttgactgttcgagtttccggttactgcgtctactttgcagacctgaccaaagaacagcaagacgatgttatcgctcggaccttcttcgacgaaatg

SEQ ID NO: 38 - DNA, 1341 bp, Lactobacillus rhamnosus
atggcattttcaaaagaaacccgcacccagacgattgatcaattgaagcagaccgaactcgacttactcattgttggcggtggtattaccggtgccggcgtagcaattcaggcagcagcaagcggtttaaaaaccggcttgattgaaatgcaggatttcgcggaaggaaccagttcccgctcgaccaaattggttcatggcggcattcgttacctcaagacgtttgatgtcggcgttgtcgccgataccgttaaagaacgtgccgtagttcaaggaattgcgcctcacattccgcgaccattcccgatgttgttgccgatttatcaggaagccggcagtacttttgacatgttcagtatcaaaatcgccatggatctctatgatcgtctggcaaacgttgaaggttcccaatacgccaactacaccgtcaccaaagatgaaattctgcaacgtgaaccgcatttagcctctgatggcctccaaggcggcggcgtgtacctcgattttgtcaacaacgatgcccggcttgttattgaaaacatcaaagaagcagcagaattaggcggactgatggctagtcgggttcaagccattggcgttttgcatgatgatgcaggtcaggttaatggcttacaggttaaggatcttttggatggcagcgtttttgacattcatgccaaactcgtgatcaatacgaccggaccttggtctgacaagttcaaggcgttggatcaagccgaagatcaaacgccaacattgcggccaacgaaaggggttcacttggttgtcgatggttctcgactgccggtaccacagccaacgtatatggatactggcttgaacgacggccggatgttcttcgtggtgccacgggaaggcaagacttacttcggcaccaccgataccgattaccatggcgatttcaaccatccgcaagtcgaacaagccgatgtcgattatctcttgaaagtcatcaacaagcgctatccgcaaagccatatcacgcttgacgatatcgaagcgagctgggcaggattgcgaccgctgattgccaacaacggcagctccgattacaacggcggcggtgcgaacaccggtaaagtttcggatgattcctttgaagcgttaatccgtgtcgttgatgattacgaagacaaccaggctacccgcgctgacgttgaacatgcgatctccaaactagaaacagcccacgccgaagctgctttgagcccatcacaggtttcccgcggcagctcacttcgccaagccgatgatggcatgatcaccttgtccggcgggaaaatcacggattatcggaaaatggcagcgggcgcgcttgct

SEQ ID NO: 39 - DNA, 726 bp, Lactobacillus rhamnosus
gatctcttctgcccagatatcacggcagatattctgactcgtaaagacgatcttggtagtgacaagccgatcgttgatgtgattttggaccgcgctggcaacaaagggaccggcaaatggtcttcacaatctgctcttgagctaggtgttccgcaaagtgtgattaccgaatccgtctatgcgcgttacattagtgcgatgaagcaggagcgggttgcggcaagtaaagttctgccaaagccggtcggaaatgtcacgattgacaaaaaagaagctatcgagatgattcgtaaggcgttatacttcagcaagctgatgtcctatgctcaaggctttgaacaaatgcgcgttgcatcggataactacgactggaacctgcagtacggtgaattggccaagatttggcgtgcaggttgcatcattcgcgcacgtttcttgcaaaatatcaccgatgcctacgataagaagccagatttacagaacttgttgttagacgattacttcctgaatattgctaagaactatcaggaaagtgttcgtgacttggtcggcttggcagttaaagccggtgttccggtgccgggcttctcagcggcgatcagttactacgactcttatcgcgcccctgttctgccggccaacctgactcaggctcaacgcgactactttggtgcccacacatatgaacgtactgatcgtgatggcattttccattacacctggtac

SEQ ID NO: 40 - DNA, 972 bp, Lactobacillus rhamnosus
gaagatttctttatacagataagcgctacccagcatcactgaatccccgactgctgtgatcagattccgaccggtgacttttcattcttcgataacactttggacgttgcaaatcttttaaacattgtacccaagcgctaccaagacctgaacttatcaccgcttgacacctactttgcccaagcgcgtggctatcaaggagaggccggcgatgttaaagctctggcgatgaaaaagtggttcaacaccaactatcattacttggtacctgaattcgatcgcgataccaagatccaagtaacggattggcagcttttcgtgcaatttgaagaagctaaagcgctaggcattaacggacgtccgactttgattggaccgtatacgttactgaaattgtctcgcttcattgatgttgtgcctgatgactttgtagccgacctgatttctgcgtacacgaccatcattgatcgcttgcatgacgccggagcagactgggtacaacttgacgaaccggcgctggtttatgatcaaaccgatgccgacctcgccttattcgagcggctttataccccgattttgacccaaaaaaaagctgccaaaatcctggttcagacttattttggtgatttaaccgattcgtttgaccgtattcaaaagttgccatttgacggcttcgggctggattttgtcgaaggatatgccaatcttgatctgctcaaacaacacggcttcccagcgcacgctaccttatttgccggaatcgtgaatggtaagaacatttggcggacacattatgccgatgccttggcaacgatcaaacaactggcaaccattacggacaagctggtattaagcacctcgacctcactcttgcatgtcccatatacacttcgcaatgaaacccatctgaaacctgaagaaaagcaatatttggcctttgccgaagaaaaactcaacgaattgcatgagtta

SEQ ID NO: 41 - DNA, 888 bp, Lactobacillus rhamnosus
gggccggcgattttcggctttattccgatgcaggatggctcgcccgcgccggggctgagtaatatcacggcagaaggctggttcccgcacggtggcttaccgattttgatgactatggtggcagtgaactttgctttttcgggtaccgagcttatcggcattgccgccggtgaaacggaaaacccgcgcaaagttatcccggtagcgattcgtactaccatcgcgcgactgattattttctttatcggcaccgtgtttgtgctggcagcgctgatcccgatgcagcaggtgggcgtggagaaaagcccgtttgtgctggtatttgagaaagtagggatcccgtacgccgctgatatttttaacttcgtgatcctgacggctattctttgtgcagcgaactccgggttatatgcctccgggcgcatgctgtggtcgttgtcgaatgaacgtacgctaccggcctgttttgcgcgagtaacgaaaaacggcgtgccactgacggcgctgtcggtcagtatgctcggtggtgtgctggcgctgttttccagcgtggtggccccgaacacggtatttgttgcgctgtcggcaatctccgggtttgcggtggtagcggtgtggctgagtatctgcgcctcgcattttgtttttcgtcgccgtcatctgcaacaaggtaaggcattgagtgaattacattatcgcgcgccgtggtatccgctggtgccagtattaggttttgtgctgtgcctggtggcctgtgttgggctggcattcgatccagcgcagagaattgcgttgtggtgcgggttaccgtttgttgcgttgtgctatggtgcttatttccttactcaaccccgaaacgcaaaacaggagccagaacatgtcgcagaa

SEQ ID NO: 42 - DNA, 1422 bp, Lactobacillus rhamnosus
atgcgtaaacaattacccaaggactttgtaatcggtggcgcaactgctgcttaccaagttgaaggggcaaccaaagaagacggaaaaggtcgagttctttgggatgattttctggaaaaacaagggcggtttagtcctgaccccgccgctgatttttatcatcgctatgatgaggatttggcgttagcagaagcatatggtcatcaagtaatacggctttcaattgcctggtcgcgaattttttcggatggtgccggggcggtggaatctcgtggcgttgctttctatcatcggctctttgctgcctgtgccaagcatcatcttatcccgtttgtaacgttgcatcattttgatacaccagaacggttacacgagattggtgactggctgagtcaagaaatgctggaagattttgtcgagtacgcgcggttttgcttcgaagaatttccggaaatcaaacactggattacgatcaatgaaccaacgtccatggcagtgcaacaatatacgagcggtacttttccaccagcggaaaccggtcattttgataaaacatttcaagccgaacataatcaaatcgttgcccatgcgcgtattgttaatttgtacaagtcaatggggctagacggtgaaatcggtatcgtgcatgccttgcagacaccttatccatatagtgattcgtcggaagatcagcatgccgctgatttacaggatgcgttggaaaatcggctgtatttagatggcacactggcaggagattatgcccctaagaccttggctttgatcaaagaaattctggcagccaatcaacaaccgatgtttaagtacactgatgaagagatggcggctattaagaaggcggcgcaccagcttgattttgttggcgttaataattacttcagcaaatggctgcgcgcttatcacggcaagtcggaaacgattcataatggtgatggctcaaagggatcgtcagttgcccgccttcacggtatcggcgaggagaagaagccagccgggattgagacaacggattgggactggtccatctatccgcgtggtatgtatgacatgttgatgcggattcaccaagattatccgttagtaccagccatctatgtcaccgaaaacggtattggattgaaagaatccttaccagcagaagtgacgccaaatacggtcatcgcggatcccaaacgcattgattatttgaaaaaatatttaagtgccattgccgatgcgattcaggctggcgcgaatgtaaaaggctactttgtctggtcactgcaggatcagttttcctggacaaatggttatagcaaacggtacggattgtttttcgtcgactttccgacgcaaaaacgttatgtcaagcaaagtgccgaatggttaaaacaggttagccaaacgcatgtgattcccgaa

SEQ ID NO: 43 - DNA, 774 bp, Lactobacillus rhamnosus
atgacgaccttgaaatcattccgggtgatcaataaggttgacttgcctagtgcccagcctgatgttgttaaagaagagattgaagagatgatcggcttagacgcttctgacgccattttggccagtggtaaaaccggcttgggagttcctgaaattcttgagcgcatcgtctcagacattccggctccttctggcgatgttaacgcgccgttgcaagcgttgatctttgattccgtttatgatgattatcgcggtgttgtccttgatgttcgggttaaagaaggacaagttaaggtcggcgatacgatccagctgatgagcaatggcaagcagtttcaggttactgaagtcggcgtgatgtcacctaaagcagtgaaacgcgattttctaatggtcggggatgtcggttatatcacggcctcaattaagacgattcaggatacgcgcgtgggggacacggtgacgctggcagaccgcccggcagcagccccgctgaaagggtatcgtaaaatcacgccaatggtctattcaggcctgtttccagtagataacgcgaaattcaatgacttgcgcgaggcattggaaaagttgcaactcaacgatgctgcgcttgaatttgagccggaaacgtcccaggcactcgggtttgggttccgttgtgggttcttgggcctcttgcatatggatgtggttcaggaacggctggaacgcgattacggtctcgatttgatcatgaccgcaccgagcgtggactatcaggtagcgttaacagat

SEQ ID NO: 44 - DNA, 1254 bp, Lactobacillus rhamnosus
atggatgtcaccacgattgatctggaacaaatgggccgagcagcaaaggccgcggcgactgtcttgagccagttgacgaccgcacagaaaaatgccgggttgttggccatggttacggctcttgaaacgcatactgaaacaattttgggagctaatcatgaagatctaaaagcggcagcaagcttgccggctaagttcacggatcgattggtactgacagccgagcggattgctgacatggcagcaggggttcgccaagttgccgccttacctgatccaaccgcccagacggataaggcctgggtgaatcacgcaggactgaatattgcgcaaaaacgggtacctttaggggtggtcgggatgatttatgaggcccggccaaatgtaaccgttgatgctgctgcgttaacttttaaaagtggcaatgcggtcattctccgtggcggtaaagaggcgctgcacagcaatttggccttggcgaccgttttacaggctgcattgaccgcacaaggattgccaaaagacgcgattcaattaatcacggacccgaagcgagaagtcgcgaatcagatgatgcacctgaatggctacattgatgtgctgattccgcgtggtggccgagggttgattaaagcagtcgttgaacaggctaccgtaccggtcattgaaaccggggcgggcaattgtcacatttatgttgatgcgtatgcgcaagcccagatggcaatcgacattgttgtcaacgccaaagttcagcggccgtctgtttgcaatgcggccgagaaacttttaatccacgctgatgttgcaaacgcgcagctgcctttaattgctgcggcactgcaagcgcatggtgtcgaattgcgcggtgatgaacgggcgcgggcaattgtgccgaacatgcagatcgccacggaagaagactgggataccgaatataatgacttaattatggcggtcaaggtggtggattccgaggaagaagcgattgcgcatatcaacgcacacaacacgaagcacagcgaggccatcattacggataactaccaaaatagtcagcaattcctccaacaggtagatgcggctgttgtctatgtgaatgcctcaactcggtttacagacggcttcgagttcggtttcggcgcagagatcggtattagtacgcaaaaattacacgcacgcggaccgatggggttagcggcgttgacgacgattaagtatcaggtgctgggtaacggacaggtacgcgaaggt

SEQ ID NO: 45 - DNA, 489 bp, Lactobacillus rhamnosus
atgaccgcatttttatgggcacaggatcgcgatggcttaattggcaaagatggtcatttgccatggcatttaccggatgatttacattatttccgggcgcagacagttggtaagatcatggtcgttggccggcgcacctatgaaagttttcctaaacgtcctttacctgagcgaaccaatgttgttttgacccatcaggaagactatcaagcgccaggtgccgtggtcgtgcatgatgttgcggcggtttttgcttatgctaagcagcatcccgatcaggaactggtcattgctggcggtgcgcaggtctttacagcgtttaaagatgatgtcgatacgttattggtgacacggttggctggcagttttgaaggcgacacgaaaatgattccattaaactgggatgattttaccaaagtctccagccgcaccgttgaagataccaatccggcgctgacgcacacttatgaggtttggcaaaagaaggct

SEQ ID NO: 46 - DNA, 285 bp, Lactobacillus rhamnosus
caagggtgcaacctttacggtatcgcaactgctttggcacggatttccaaagcaattcttaacgatgaaaatgcggtactcccattgtccgtttacatggacggccaatatggcttgaacgacatctacattggtacacctgctgtgatcaaccgcaatggtattcagaacattctggaaatcccattgaccgaccacgaagaagaatccatgcagaagtcggcttcacaattgaagaaagttctgaccgatgcgtttgctaagaacgacatcgaaacacgtcag

SEQ ID NO: 47 - DNA, 969 bp, Lactobacillus rhamnosus
atgttgacgaaacggcaattgctcgtcttaaaagaaatcatccgcctgtttactgaaagtgggcagccggtcggttccaagacgttaatgcaggaactgccggttcatgtcagttccgccacgatccgcaatgatatggcatcgctggaagacgccggtttgatcaccaagactcatagcagttccggtcgagtaccctcgactcaaggctatcgctactatcttgatcatctggtcgagccagtgcgtgtttcccaccgtgaactagccacgatcaagcaagcattcggtcaacgctacaataagatggatgaaattgtggcgcaaagtgcgcagattttatccaatctgaccagttacacggcgatcagcttagggccagaagtgaataacattaaattgaccggatttcgccttgtaccgttgggcaatcaccaggttatggcgattttagtgacgaacaacggcaatgttgaaaatcaggttttcaccgttcctgaaagcatctcatctgacgagctggaaaaggcgattcgcattgtcaatgatcagctggtcggtttgccgcttattcaagttgctcagcggctaaagactgatgttccgtcgatgctgatgcagtatctgaccagtcctgaaggcttcctggatatcttcggtaatgtcttaaagtccgccgcttcagagcgtttctatgtgggtggccgcttaaatttaatggactatctcggcgactcggatattcacgagttgaaaaagattatgtccttgattgatgctgatcatggtgatcttactgaactgcttggcggaccggttcgccaaacgccggttacggttcgtctaggcccggagttaaagccaattgatctggccaatctcaagctgattaccgccagttatgatgtcggtgaccacggcacgggaatgattgccctattagggccaacccaaatgccgttttccaag

SEQ ID NO: 48 - DNA, 1336 bp, Lactobacillus rhamnosus
ctaattcaccgtatacagcggcgaataaactgaagatgacggtggctaagacgctggacaagtttcagctggatgtgaccacaggcaacgggcttaaggcgattaacattttcatgaatgacagcacgaaggaaaatgttgaacaatatgaatattggattaacaatttcattgaacgcggcgtgcttgagccaaaataagtctctttgaaaatcacggtaactctcctttcgaacaagcggagattaccgtgcttttttgttgtatgaatcacggattaaagcgttacgactggtaaagcatgagctacatcacggatgcgtggtttaagtgaggcaaatgtcgtgaccgccaaatcgagcggccaaaagtggtaagctggaaacattgaaattttgatatgaaggagggcttgtatgtttgaacatgggtttattgaggtacacgatgccaaccagaacaacttgcaacatgtgaatgtgaagatacctaaggatgccattacggtttttgtgggtcggtctgggtcaggcaaatcatcgttagtgttcgatacgattgctgcggagtcacggcgggagttgaacgaaacttttccgagctttacccagcaatatttaccaaagtatggccagcctgatgtcggctcgattgatcacttgccggttgccattgtggtggagcaaaaacgtatcgggaaaaacgctcgctcaactttagcaacttacacgggcatttactcactgttacggctgttgttttcgcgtgccggcaagccattcatcggctattcggacacattttcgtttaatttacctcaagggatgtgccccacctgccaaggcttaggttacgtagatgatattgacgtcagtaagttgattgatcccaataaatcgcttaaccaagaggcgatcacctttgtcagttttggaccggatacttggcgttggcggcgttatgcttacagcgggttgtttgataatgacaaacccttacgtgactatacgcccgaagaaatgaaactgttactttatgcaccgcaacagacactgaagcatgcaccggctaaatggccaagaacagcgctatatgaaggtgtcgtgcctcgcattaaacgatccattattggtaaaaaagaagcagaacatcataaggccgcactggcagaaatcgtaacgcgcaagccttgtccggattgccaagggacacgcctacgtccggaagtgttaacctgtttgattaatcaaaccaatattgcccaagtgctgcaaatggacttggtaaacgtacggcattttctaaaaaacattcaagtgccgctggttcaggatgt

SEQ ID NO: 49 - DNA, 760 bp, Lactobacillus rhamnosus
aatgacacaatcggccgatccccacgcgccgccattgcttgcaaagtggcggcaatgggatactgaccgccataagtcatcgccatttgaacatcccaatccggaagtgcctggcgcaagcgatcgtttattaactgaagagattgcgggcattttcattttaggaacaaatggcgagtcttacgtcttagcggaagatgaaaagttggcatttgtagaacatgttatcgactatgtccatggtcgaaccaaagtactggtaggaacaggtttgaacggaacagcggaaaccattcgcttcagtcaaaaggtagcgtctttaaaaccagacgcaattaccttagttgctccttcatttgttgctccctcgcaacaggaacttgttgatcacgttgctgccataattcacgcggacgatatacctgttctactgtacaatatgccagcaaaaacgggcattaacattgagccagcttcattaaaacagttgtcaaaatatgagaacttaatcggtataaaagatagctcgggaaagtgggagaattttgacggttatctagccaatcgccccgaacgaccattctcagttattatgggctctgacggtcgcattcttgaaagctttcagcacggtggtaatgcggctattgcaagtacagccaatctcctgacggctaacaatgtagcgttgtatcaagcatttgttaatgacaatattgaaaaagcccagaaatttcaggatcggattcagccccttaga

SEQ ID NO: 50 - DNA, 1056 bp, Lactobacillus rhamnosus
aagacagttgccaaaaccccaatcacgagactgacaagaacggctaatacagcagccagcgtacccgtagttggccaataaggcgcaatccccgtttcaatcagaatgaccaccgccgcggcgacaatgccacaaaccgcgtaataaacctcaacctcaaaatgttttagtaaataacccattagtttggcaatggcaatgagcccgatcaacgcacctaccataaaaggtaccagtaaggcgacagatcaaggtgtcgactggagccggtaccaaggagataacggtgtctttggttactccactgacaagttcggcatctctcaaatcggtggctatagcggctacggcacgtacgagcaaaccacgtataagacacaggttgcatcgttgattgccgctggcaagcgagcacacacctatatctggtggcagaatatcgacaacaccaattttgccaagcaagtactagatcatttcttgccagagattcaaacaccaaaagggtcgattgttgcgcttgattacgaggccggttcaacaaatacggcaactttgctgtgggcactcgactatatccgtgatgctggttacacgccaatgctgtacggctataagagcttcttgatgagtcacattgacttgtcacagattgccagtcgctaccagctatggcttgcggaatatcctgattacaatgtcactactgttccgaattatggctacttcccgagttttgataatgtaggtatcttccagttcacttccacctatcgcgctggcggccttgatggcaacgttgatcggtctccgcgaacgctcacaaaacctcgctgttgtcaatgaagctatcgagaacacagtttcccgtccagcttacgtaacctttgacgacaataagaaggaaggttcccttgttcgcttaccagaacgtggcgaactcgaaccagaagttgacgaatcactggttgttgaatactataaccagaaactttaatacttcacagctacagccaactctttgcggagttggctgtttttatttggaatcagatc

SEQ ID NO: 51 - DNA, 1310 bp, Lactobacillus rhamnosus
cgccggtgaacgcgtttcagaccagagcgtgaacaggagcgcttagaaatcggagtgtaagtggcctcagacgtgatgacccgggctttggccattgcgttccaggtctttacacgcagacccatgcgccggtgaacgcgttttagaacgtgaaccggcgcgcacacgtaaccccccctaaaaaaccaccaccccaaacctaacgctccttagattcaaacctccaaacacctcacttggcactccccatttacagcattagaatccgccctgtttttaaggtatactaaggcgtactagaccaaaatttaaggtgggacaagcacttgaaaaagttcgtcaatcgggtcaagacgcttgggactcgattttgtcgctggttcacgcaattcgtgacccggcatcccgattccaaatcagataccaatcaacaactaacgggtaaagctgccgttgtctattatggcaacgtcacacttcaaagcatcaaaacaactgtgttactatttattaggcgttttaggcattgctgtcgtgtttggccttggcttgtttggcggctattttgtgtcgattattgacgcaacgccaattccaactgaaaccgctatgaaagcaacgttgtccaataccagccgcacttccagcatgtattttgcccacaacgtcaagcttagcgatgttaaaagcgacctgtactcaaccaaagtcaacctcaatgagatgtcgccatggctcaccaaggcgattatcgccactgaagatgaagacttctatcgccacaacggcatcgttcctaaggcggttatccgtgcgttcttctccgacttaaccggtatggggagtcagacgggggggtcaactttaacgcagcaagtggtcaaaatgatgtttttaaattcggagacaacctttaaacgtaaggctgctgaaattatgctggcccggcgcttgaacaatcacttcagtaaaaacaccattctggcaacttatctcaatgttgctacgcttggtcgtaataacaaaggccaaaatattgccggagttgaagcagcggctcaaggactgtttggggtttcggctaaggaagttaacctgccggaagcggctttcatcgctggcctgccgcaaagtccatttgtctataccccttacacggccgatggtaaattgaaaaccagtctcaaagccggtatcaatcgccaacaaaccgtcctgttccgcatgtatcgggctggcgttatcagccatcgtcaatatgttgctgccaaatcatttgatccactagtgtcgacctgcaggcgcgcgag

SEQ ID NO: 52 - DNA, 1859 bp, Lactobacillus rhamnosus
ccttgcagcgatccggttatggggtttatgttccgcttttttccgggcatgggacagtggagccgttagatattttgacaaaaggcaacccggatatttggtgggcagaaagtagtgccgcggttgcgcatatgaccgcaaaatacgccaaggtgtttgtttttggcttatcactgggaggtatttttgcgatgaaggcgctagaaaccttgccagggattacagcaggcggtgttttttcatccccgattttgccgggcaaacatcacttagtaccgggttttttaaagtatgccgagtatatgaatcggttagcaggcaaatcagatgaaagcacacagattctggcatatttgccgggacagttggccgcaatcgatcagtttgccacgacggttgctgctgatttaaatttagtcaaacagccgacttttattggacaagccggtcaggatgaattagttgatggtcgattagcgtatcaattacgcgatgccttaatcaatgctgcacgcgttgattttcattggtatgatgatgccaagcatgtcattaccgttaactcggcccatcacgcattagaagaagacgtaatcgcatttatgcaacaagaaaacgagggatagcatgaccacagttggccatattcgtaatgaactattagcaacatttcgtaagaatccgaacattgattattcggttcaaacactcagtcgcgcacttaagttaagtgaaggcggcgattttaaagtactcgtccaggcgttaaacggtatggaaaatgataacctgattcacgccaatcacgaaggacgttatgcattgggcggcgcgcctaaagtcttgaccggcactttccgcggcaacgaaaaaggctttggcttcgtggcagttgagggcttggacaatgatgtttatgtaccggcgatgaacaccgattttgcgcttgatggcgataccgttgaagtgcggatcgttcgtgaagcccgtcccaatgatagtcgcggacctgaaggcgaaatcactaagattgtgcagcgcagtttaaccacactggttggtgaattcaaaccattttccgataaagatcgggctaagtctggatttatcggaatggtggttagtcatgaaaagaaactgaagaattttccggtttatgttaaagataccggtaatattccgcaactcggcgatatgaccgtgacggagattactgaatttccaaccgaatatcatcccaagttgatgtatgggatcgttgtcgagacgttaggcaacaagaatgatcctggcgttgatattatgtcgctggtcatgcaaaaccatatcaaaacggaatttccggatgaagtgatggatcagaccaatgccattcccgatcacgttacgccagaagaacgagttggtcgcaaagatattaccgatcaggctgttgtcacgattgacggggatgacagtaaagactttgacgatgccgtggtggtttggaaattaccaaatggaaacttccatcttggggttcacattgccgatgtttcgcattatgtgacggaaggctctgcgttggatcaagaagcatttgatcgtggtaccagtacgtacttggttgatcgcgtcattcaatgctgccatttcgactatccaatggcatttgttcgttaaatccaggtgtagatcgattggcaatgtcatgtgatatggaaatcgatcatgatggtcatgtcgtaaaccacgagatttatcaaagtgtgatcaagagtcatgcccggatgacctacaacaatgtgaataaaatcgtgaccgatcacgacccggaagtcatggccgaatatcaagaactggtacccatgtttgaagac

SEQ ID NO: 53 - DNA, 887 bp, Lactobacillus rhamnosus
cttatgaaaatgaaattatcatcacgattcgagctggacgcaagaaccatcccttgttgctctcagctaatccacagtatgcgcgggtgcaaattacccacattccatttacaaatccagacgttcctgcaaccttcacgatgacgttgcggaagtattttaacgcggctacgttaacagagattcaccaagtgcaaaacgatcgggtactacactttgaattctccacgcgggatgaattgggggatgaactggggctgcgcttgatcattgaaatgatgggtcggcacagtaacatctttttagtcagcaagcgcaccggcaaaattattgatctcattcgccacgtttctgcggatcaaaatcgctatcgtccgttgatgcccggtgccccgtatgtcgagccgcctaagcaagataaagtggatccgtttcatgattcggagcggatttatcacgaacttgaacgtcaggtaacaccttcattgagtcgcgccgccttgctccagcaacattaccaaggacttgccaaggattctgcagctgaattggccctgcgactcaatcaaggcgatgccggctgggatagcttttttgcagcgctggcaacccctgaaccgactattacaacccaagggaaaaaagccgtttttaccgcgatcccgtatcagtctctgaccggcgagcagcaacattttccaaccttaagcgcgatgctggatgcctattatgcgcaaaaagcggaacatgatcgggttttgcaacaaggcgggaacctgattcatgtgatcaaaaatgtgattgataaagatcgcaaaaagcagcgcaaattaaagcgaacgctggaagaaaccgaaaaagccgatgattatcgaattcgattcaag

SEQ ID NO: 54 - DNA, 999 bp, Lactobacillus rhamnosus
gcctatttgggattgacagaaaaacaaaccggcagcctgtatcacctgagcgaaggccagaaaaaaatggtgcagctgatcgcaatgttaagtctggagcgaacttttttgctgttggatgaaccgtttagcggcttagatgaacgtgcctgtgcattctttgccgcgtggatcaaggaaaaagcagctaagcaggcgtttttgattgtgacgcaccgactggctccgctagctggcatcagtcaccagcatgtggcacttgccgatcaccggttacacattctacaggagtgacatgatgccagtaaaaaagaccaatgcagtgaatctgagtttatttattctactgctgacacttgaaatatctttcagccatgcggtgagtcttaatgtagcgttgatcggactggcaagcggttttttaatatggcggcgggcgtttaaaagtctcgtcgttttggccttgttaccgttgatcccggctgccagcacgtactgggcaattaccctgcatggtacggatacgacttacgctttgctgctgtgggttcgcacctatgctttcaccgcgttgggattggtgtttcttatcggagttgacttagagaccttgctgttatggctggagcagcataaattatcccctaattttgtttacggattactggttgtgatccatgccttgccgcaaatcatgcatgaagtggctgctattcgtgaagcgagtctgttacgtggccaaaagttgcacgcgtggtcgccgatgatttatgtgaaggtgatttttgttgccatgtcgtggcaggaccagtacgtcaaagctatgtatgcccatggttataccgaaggagcagcgcgaacggttcaccaaaccatccgtagttcatggcgcggcttgatcgccatggtggggggatttgtccttttaaatctaattgaccgttagtttgaggatcaaaaagaggtggtcgcgcggtgtttaagcgattgtgcagggac

SEQ ID NO: 55 - DNA, 846 bp, Lactobacillus rhamnosus
tttgctttacaaaaaccagcatagcgatcattccttaattaccagcataatcaatccttaattaatcctaaattgtgaaaaggtgcattaatagcttacactatcattggaggaaaggagtgacgatgatggcgaagatattaattgttgaagatcataggatatccaggcacttattgaaagatgtactaaccccgacttatacggtcactcaggcttatgacggcattcaggctttaacggcctttcatcgagaacaaccggacttgatcattctcgatttaatgctgcccaacgtgactggtgaaagtgttttaacaaccattcgtaaaacatcccaagttcccgtgctggtgttaacggcgattcaggaaaaagccaaaaccgttgccctgctgcagcaaggcgcaaacgattatttgaccaaaccgtttgacattgacgaattactagcacgcattcaagtccaactgcgccaagtcagcggccaaccaataacgacgaacgatcaactaaaagtcggcgaaattcaattagaccctaagcgtcatgtggtgaccgttaatcagcaaaccctaacgctgcctaaaaaagaatatgacatgttggcgttaatgatgcgtgatccccatcaagtctttgataaaagccaactttatgaacatgtatggggggagccgtttttaaatgccgataataccctaaacgttcacatcagcaacttacggacaaaaatcaatgaacttgcccatgatcccaaatacatcatttcaatctggggcatcggtgtacgtttgatttagcaaggagaaatcattatgcttggcttccttattttacttatcg

SEQ ID NO: 56 - DNA, 780 bp, Lactobacillus rhamnosus
tacgatttgcttcagttcctcaagatccggataacttggcaggaattgattccaaccgcattgccaagtatcaagaagcgttcgcaaaagcctacaaacgactcatggaagcaatcagttccatgagcattagctggacgattatcggtgctgcaagtccgcgctgggctcaaaaagttttccctgatgccgccacccctgaagaagcaactgagctactctgggaggcaattttcaaaactacccgaatcgatcaacccgatccggaagctgcctggaaagcccacgatcaaaagctgcgggaaaaagcggcctggttaaacaacgaacaatttgatcagctgcattacatggctccgggaacggatttggtagtcggtttaccaaagaaccacatttgggaaggcgccggcgcttttaacccgcgtggtgaggaatttatggctaacatgccaaccgaggaagtcttcaccgcccctgattttcggcgcatcgacgggaccgttgcttccaccaaaccgcttagttatggcggcaacatcctcgaagatatgcactttactttcaaagacgggcaaatcgtggaagcccacgccaaacaaggcgatgacgtcttacaaaacctgctaaaaacgccgggtgctcgttcattaggcgaagtgtcgttggttccggatccttcttccatctcacaatcgggccttattttcttcaatacgctggttgacgaaaatgcttccgatcatatggcactcggtcaagcctatccgttctcagtca

SEQ ID NO: 57 - DNA, 1569 bp, Lactobacillus rhamnosus
ttgcatcccgctctgctgcctagttttcccggacggcagggcattaaagatgcgtttgattatggcgtcaaagtgaccggcgtgacggttcattacgtagatgccggaatcgacaccggcgaaatcatcgcacaggatccggtccgcgtaagcccggggatgacgctggcacaattagaagcagccattcaccatcaggaacatcaaacttttcccgcaactgtcaagcaactcattgaagaaggagcgatttaagtgaagcgtgcattattaagtgtctctgataaaactggtttggtaccttttgcaaaaggtctggttgaacgcggttttgaactgatttcgaccggtggtacccatcgcgcactggctgaagctggggttgcggtgaccggcgtagaagcagtaaccggttttccagaaatgctcgatggtcgagttaagacactgcatcctaagatccatgccggcatcttggcgcggcgggatgatcctgcccatatgcaggcattggcagatcatgatattcagccaattgatgtggtttgtgtcaatctttatccgtttgccgcgaccattcagcgtgcgggtgtgacgcgtgccgaggcgattgaacaaattgatattggtggcccgtctgcgttacgtgcggcagctaaaaatagtgacagcgtctgggccgtggttgatccggcagattatgcggatgttttagccggactcgatcaaaatgatgctgatttacggcaacggttggcagccaaagtcttttccgccactgccgcttacgatgctcaaatcgcccattacttagatccagagcctttcccagagcagttcacgccaacttatcacaaacggcaggacttgcgttacggggaaaacagccaccaacaagctgccttttatgttgagccgaatcctgatccaaccagtcttgccgctgctaagcaattacacggtaaagagctttcttataacaatatcaaggatgcggatgcagcattggcaatgttgcgggaattcaaacaaccggcagccgtggccgttaaacatatgaacccatgcggtattggcttgggcgatacgcttgaagccgcttgggacaaggcatatgccgccgatccgatgtccatttttggcggcatcattgctttgaatcggcgggttgatcttgccactgccgagaaaatgcataaacttttccttgagatcatcatggcaccggcatttgatgatgatgcttatgagattctggcgaagaagaaaaatgtgcgcttattgacgattaataccgccgatacgccagaagaattgggaacagaaacaacttcaatttatggtgggttgttgattcaaacacgtgacgacaaagctgagacaccagccgatatgacggtggtgacggaggtcaagccgactgaagctcaactcaaggcattggcatttgctcagacggtggtcaaacatgttaagagtaatgcgattgtcgtggctcaggctgatcagacgttagggattggcgccggtcaaatgaatcggattggctcggttgaattggcgttaacc

SEQ ID NO: 58 - DNA, 1112 bp, Lactobacillus rhamnosus
gatcgtgtcagtggctttagtggcaggttgtgttggcctgcatcgattcgttgctgaacttgttcttccgataacccttgatcaggtgaaatcttttgctctgccatcgcatgccctccagtacttttcatccattttaccaatccagacaacaagatgcccgaatatcacacttgtttttacaaaactttgaagttgttggcgtggttgggttatggttagggagtcatgaaaccgtcataatgagatgaaagttatagaaagaagtgctctcatggtaaaacgaaacccaaatggaacccgatttatcacattacctaatggctaccacttgtggacccagacattagcagcggccgattcattactgacgttgcacggcggaccaggcggcacgaatgaagtgtttgaaaatttcgctactgaactggcatcttttggtgtccgtgtctcacggtacgaccaactgggttcatttttctctgatcaaccggacttttccgatccggctaatcaaaagcgttttctcaatatcgcctattatgttgacgaagtggaaaatgttcggcaacagctgggccttgatcatttttacctattaggtcagtcctggggcggtgtgttggcgattgaatatggcttgaaatattcgcagcatcttaagggacttattttgagctcgatgattgataatttggatgagtatttggttaacattaacaagattcgggagaccatgttttctagtgatgacgtggcatacatgcaacggattgaagcccagcacgcgtttacggatgccaaataccagcaattggtgcgtgagttaggggagcaatatcttcatcatgccaaagatccccagccgcgccatttaattagcacgttggcgacccccgtgtatcatcattttcaaggtgataatgaatttgtgatggtaggcgcacttagggactgggatcgacgggctgatatacaccgtctgacaatgccgacttatctgacattcggtggacatgaaaccatgccgttatcagctgccaagcgaatggctcggacaataccaaatgccactttgcatgtcacacctaatgccggtcatggtcagatgt

SEQ ID NO: 59 - DNA, 1570 bp, Lactobacillus rhamnosus
ataggggatgagtaggcgatataggcagtttgatgctaaccgcgccggctcgcgctcacgaggaggattaaatttgatttcactaggaatttacgaaaaagcactgcccaggaccgaatcttgggttgaacggttgaaaatggttcgggatttgggtttcaactttttggagttatcggttgatgaaagtgacgaacggttggcccggttggattggacggcagcgaaacgggcgaaggtgcgtgatgcttgttggcaaacaggggtgcggatccatacattgatgttaagcggccatcgtcggtttccattaggctcggcagacccggcgatccgtgaaaaaagtctgacgatgttatgtaaggcgattgatttggctagtgatctgggcgttcgcaatgttcaactagccggttatgacgtttactatgagccgaaaaccttagcttcacgagaatatttcatcgagaatttgaagcgcggtgtggcctatgctgcggctaaagaagtgatgctggcaattgagacgatggacgatccgtttttgaattcactttccaagatcaagacgattaaagatgagattccgagtccgtggttgcaagcatatccggatctgggcaacttgtcagcctggccggaaaacaatgtcggtcgcgagcttgaactgggaattgcaaacatcgtctcggttcatttgaaggatacccaagcggtgacggtaaaaagcaaagggcaattccgcgatgtcccgtttggcgctggcgtggtggacttttccggctgcctgcgtacgctcaaacgccttgactacagcggtgcctttacgatcgagatgtggacggaaaaggccgctgatccaatccaagaagtgaagcaggccaaggactttttcgatccgctgtttgtgcaggccggttttgttcaggagccagtggcaaaaaccaatgtcccatcatgaagcgcctgctgacctgcacacgaaaaaagtcgccaattgcttgccggctttagacgattcatttaactataggcgaatggcagtggggctgatctggaagtaatcgattaaatagttagcaaccccgtcatcatcatttgagcccacggtaatgtccgcaatttgtttgatttcaggaatcgcgttacccatggccaccccgatgccggcatactccagcatgtctttgtcattttcctcatcgccaaatgcggccagttgttcgcgcgggatgcggtaaaagttgagtgcggcttttaaaccgttcatcttattgacatttggcggcaggatttcaagcagtgtgcgccgggatcgcgtgacggtcagctgcttgagtgactgggcagcgtctgatgcggcatcgatctaagtgcaacgcgattggcaaatcagtatgatttgccgcatcaataacttgtgccacaaaactgtcgcggagaaattctaattctgtgggatgaatcgccataataaatggtgcctgcagttcttccgccttttccaacgtggcagtgaagagttgtcctgagcctgcgttgaaagcaggaatggcaaaatggtgttcct

SEQ ID NO: 60 - DNA, 948 bp, Lactobacillus rhamnosus
aactagtggatcaaacatgacagatcccattgcgtttttgcaaaaactaatccaaattgactctgcaaatggaaacgaacttgcagtagcccgcgttttgcaagctgaactcgaagcggccgatattccaaccaaattgatcccatacaaagatgatcgggtcaatttagtcgcccagctcaatcacggtgaccgcgtattaggcttcaccggccatgaagacgtggtgagtcccggcgatgagaacgcctggacctatccgcctttttccggaaagatcgtgaacaacaccatgtacggtcgcggcaccgatgatatgaaaagtgggctagcagccatgaccttggcactgatccaccttaagcaaagcggctttgcccatccgctgcgtttcatggccacggtcggtgaagagtttggtgcaatgggagcgcgccaactcaccgaacaaggttatgccgatgacttaaccggactcgtggtaggcgaaccaacgaacaaattgctaaaatacgcacacggcggaacggttaactacgaaattgacagcgaaggcgtttccgtccacagctcccggccggaaaaaggcgttaacgcaattgaagggttggtggcattttctactcccgaaccgcacgcctttgatcaggcccctgatgatcctgaccttggtccattccgccactccattaccgtgatcaaaggcggcgatcaagtcaacaccattcctgcccatgcatacttacgcggcaacttgcgcccgactcctgcagcaaatatcgaattagtcgtcgggttattggaaaaattagtcgatcaggcaaataaagccaccgccgctaacttgacgttgaacgttttacatcgttttttgccggtacactctgacaaaaacgggcatctcgtgacaaccgctaacgaagccattgccgctgtgactggtaagcc

SEQ ID NO: 61 - DNA, 1188 bp, Lactobacillus rhamnosus
ggttagaaggaattggcgagtttgcgcagtgaatgcttttgtattccaatatcatcgccaaagcctgattaataaatcgattttgtatcttaaataataaatatgtacacgttttcatgaatatgagaacgtgttttttatttaaaaaagataaagcgcttgcataagagataaatggcttttatatttaacttgttcacagaggtaccctgtgatacacggttgttctatgatgttcgtaaactaatagaaagatggcgggaaaatgaaaattgatattgacaaaacgtctatgattccagtttacgaacaaattgcaaatagtttgcgagacatgatgtatggcggaagtctacaggatggagaccgtttagactctgagcagaagatgtgtcgcaaccttaatgtcagccgtggaactgttagaaaagctattgatattctactgaaggagggtatggtcaaaaagattcatgggaaaggaacctttgtcagtaacccaaacgttgagtactcgttgaatgatcagttaatgtcattcgctgagtcgctcgataatcaacacttaagttacacaacacaagttattcagcaggaactacgacccgcgaccgcgaaaattgctgatatgctcaagatccctattgatagtcagtacttgtatttagaacgattgcgatcagttgccgatgataagttaatgttaatcgaaaatcgcattaatattacgctctgtccgggaattgagaaggtcaattttaacaacattagcctttttaatgagattgaagaactagcaaaaagaaagattagctttgcccgcagtacctatgaagcacttacgattggcacggaacgtgggaaacttttagagcttccttcttccacgccggctttaaaaatgcaacagacagtgtatctttctgaaaaagaaccagtcgaatacggttccgtctggttaaaaggaaataagtactttctcacaacaactttgcaaagacgataggaggaattgaaccaatgccattagtaaatggattcgatttgatcaaaattattaaggatcggcacgttgtcgcaggagctttcaacacaactaatctggagacaaccatgggtatacttcgagcggttgaaaaaagcggcataccatctttcattcaaattgccccgacaaacatccccgtt

SEQ ID NO: 62 - DNA, 1637 bp, Lactobacillus rhamnosus
ctagtggatcccttcttatttacaccgcaactgaccatcgaagaagtcaaaaaagccggctgggcctacccggtattcggttatcttgatcacgaggacccgtttgcaaaactggcgagtcatattaaaaccgtcaaccctaatccgacaaaatgggcaatcgaaaaagataatctcgccgttttcaaatttgaagcgattatgaagcagttcccggacgctaccttcccgattgatgcttctcgttttattgaaaaacagcgcctgatcaaaaccgcttcagagatcaaacagatggaagccgctggtgctcaagccgatcgggcatttcaggcaggattcaatgccattaaagccggagcaaccgaacaagaagtcgccgctgaaatcgattatgccatgatgaaagaaggcgtcatgcacatgagcttcggcaccattgtccaagctggtgtcgatgctgccaacccgcatggcgaaccgatgggaacaaaactcgcacctaacgaattggttttgttcgatctgggcaccgacaatcatgggtatatgtccgatgcgacccgcacagttgcttttggtcaagtcactggtaagccacgggaaatttttgacatctgtcttgaggctaatttaaccgcgatggacgctgttaagccaggacttaaagcatccgaactggacaaaattgcgcgtgatatcattactaaagcgggctatggcgagtacttcaatcatcggctcggccacggcatcggtatgtcgacgcacgaattcccgtccatcatggaaggcaatgatatgatcgtgggcgaagattttgggatgcgtgtcagtgtccttgccagcagcagctctggtaacgcaacctatattgaaacgcctggtcacaaggtgttagtggatgccggtttatcaggcaagaaaattgaagcgctgatgaagagcatcggcagagatctaaccgatgttgacagtgtttttatcacgcatgaacatagcgatcatgtgcgtggtgtaggcgtgttggcgcgacgttatccgcagctcaacgtttatgcgaatgccaagacgtttgcagctttaccaaaaagtgtgggcaaaattcctgaagcacagctgcggttgtttgatatggggacaactttgacgttaggtgatttagatgtggaaagttttggcgtttcgcatgatgctgccgcgcctcagttttaccaatttcatcatgacggcaaggccttcactatcctaaccgacacgggctacgtttcagatcgggttgccgggacgattcgcgatgccgatgcgtatgtgatggaatgcaatcacgaccttgaaatgttgcggacaggtccgtatccgtggccgttgaagcaacggattttaagcgatcaagggcacctgtccaatgaggatggtgcggatgctttgatggatgtcatcggtttgcggacgaagcggatttatttggggcatttatcaccgcataacaacaataaagccactggcgcatttaaccgtggcgtcgttgttggcacaacaaggtctggcggtggatcatgactttcatatttatgacactgacccggcagttgccgacccattgtttgttgtgtgaagcggtttgaaagcagttt

SEQ ID NO: 63 - protein, 224 aa, Lactobacillus rhamnosus
LRGLCIGIVACEFFEIPLTPSESADNGIQKRNDVHQWLVIFRRDLLADLKHFDNGDRGSKGCVFDQADETIQWRDGRSCLRNNDFAQHQSPRQSNCISRFPLPGINRQQRGAGRFGTIRPRVKECYNSRGRGILDAMRENTRDDEAGAEENDELHQQRRAKEPNVKNGDSFCDSDQNTFTNRYARQCGYKCDDQADRKHECNWNGVFNAGCYHLRNCIGNNFPH

SEQ ID NO: 64 - protein, 475 aa, Lactobacillus rhamnosus
LICKGRSLKPFGHFIDAITVNREHVLTTAAEALIASAGDALNASHATFNVLNNSDLQFGFVENEDGETVQLSNGLYGQLIRSTNRKLRKEAFEALLRAYESLKNTFAQTLSGQVKAHNFNATAHHYKNARAAAMASNHIPESVYTTLIDQVNTHLPLLHRYVALRKKVLAVDQLHMYDIYTPLTGQPPLTYTLEQAKAEALKALAPLGDDYLEHVREIFDNRYIDVVENKGKRSGAYSGGAYDTNPFILLNWHDAVDELYTLVHETGHSVHSWYTRHNQPYVYGDYPIFVAEIASTTNENLLTDYFLTHSDDPKVRAYILNYYLDGFKGTVFRQTQFAEFEHWIHQQDQQGEPLTATSMSQYYADLNARYYGPEVARDPEIAFEWARIPHFYYNYYVYQYATGFAAASTLAAGISSGEPDAAAHYLDYLKSGSSKYAIDTMKTAGVDMTKPDYLEAAFSVFEQRLTELEKILQKG

SEQ ID NO: 65 - protein, 345 aa, Lactobacillus rhamnosus
EELGLPQLVRMSANENPFGTSVKVQQAVTNWNFTQSRDYPDGYASQLRTAVAKHLDVAAEQLVFGNGLDEVIALIARTFLSPGDEVIEPWPTFSEYRLHAQIEGATVIDVPVTETGNFDLSAMAQALTAKTKLIWVCNPNNPTGTLLSIATLTEWLRQIPKDVLVLMDEAYIEFTDDYPATSAISLLSKFPNLVVLRTFSKIYGLANFRVGFGVFPKQLVNYLQTVRLPYNLSSIAQVSAQAALADQDFVAMTRKRVQQARDSWERFLTQTGLPHTRSQTNFQFFQAPKMQASALKKRLLQQGFLVRDGLKPGWLRVTFGTEVQNTAVQRIIETFQAELTGPNAL

SEQ ID NO: 66 - protein, 369 aa, Lactobacillus rhamnosus
VHLAKRILNVAPSATLALSNQTKDLKAKGADVIDLSIGQPDFSTPKAIDDAAIAAIQAGNASFYTAATGIPELKQAISDRIFAQDGIRYDHRQIVATTGAKFALYALFQVFLNPGDEVLIPVPYWVSYEEQIKLASGVPHLVMPAVGHKVSVDDLEAARTDKTRALIINSPQNPSGVVYDRTELTLIGNWALKHHILVVTDDIYRDLIYNGTTYTSMISIDPDIAANTVLISGVSKSYAMTGWRIGYAAGPEKLIQAMATFISHTTSNPAAVSEYAAVAALTGDQQVVEKMRRAFEERLNLFYDLLADIPGFDMGDKPQGAFYLFPNIKRAAQLSHYGTVDDFISALLTETGVAIVPGRAFGHAGSCAD

SEQ ID NO: 67 - protein, 390 aa, Lactobacillus rhamnosus
MTLQPLNEQLPAIEVSEIRQFDESVSDIPGILKLTLGEPDFNTPEHVKQAGIKAIQENYSHYTGMVGDPELREAAQHFFKTKYATDYRATDEILVTVGATEALATAITTISDPGDAMLVPSPIYPGYIPLLTLNHVTPLYMDTSKTDFVLTPELIEATITANPDAKIKGIILNYPSNPTGVTYRAAEVKAIADIAAKHNLYIICDEIYSELTYGEPHVSMGQFAYDRTFIVNGLSKSHAMTGWRIGFLMGPQQLIAQAKKVHQYLVTAATTIAQRAGIEALTNGADDAQVMKAAYVKRRDFVYAALIDMGFSVARPDGAFYLFAKIPTQLHLSSREFTHALAHEQKLALISGTAFGPGGEGYIRISYAASMTDLQEAVKRLRAFMASHIG

SEQ ID NO: 68 - protein, 297 aa, Lactobacillus rhamnosus
VARLMLDPGDGLVVEAPTYLGALAAFNAYQPTYYEIPMQDDGMDINALQRVLMSHKVKFIYTVPDFQNPTGVVMSVAKRQALIRLANQYDVMILEDNPYRDLRYDGKPLPTIKSFDTQGRVVYLGSFSKILSPSLRMGWLVAAPDLLQELLALKGGSDLESSNLTMHGIDAYMAENDLDAHITEIQNCCREKKNAMVAAMNRYLPDEAHFTNPDGGFFLWLTMPAGFDMGAFMKQHLLPESNISYVPSANLYATSAQVNGARLNFTGPTLEQIDTGIKALGDALKTALQHHLVAEQA

SEQ ID NO: 69 - protein, 390 aa, Lactobacillus rhamnosus
MKLTIYDFDHVIDRRGTFSTQWDYIADRFGRNDILPFSISDTDFPVPVEVQDALKERLTHPIYGYTRWNHATYKDSIVHWFERDGHTKINPDWIVYSPSVVFTIATLIRMKSDPGDGVAVFTPMYDAFYGTIKQNDRVLIPIRLAAADEGYVIDWDSLATVLAEKQTKIFLLTNPHNPTGHVFTKSELARLYDLCQAAHVFLISDDIHRDIVYPGHSYEPMTNVGTSDVALCCSGSKTFNTPGLIGSYAFLPDHDVRAQFLTELKQKNALSSVSIFGMLAQIAAYNGSEDYVEQLTAYTKNNMELVASYLEENLPELQFSLPDATYLAWINVSKLRLTSEELQHRLVNGGHVGIMAGKTYGDTRYLRMNIACPKKKLVMGLERLKKGIRG

SEQ ID NO: 70 - protein, 386 aa, Lactobacillus rhamnosus
  VARIANT (1)...(386): Xaa = Any Amino Acid
MIYFDNSATTKISPDALATYNKVSTDFFGNPSSLHALGTKANEVLQSSRAQIAKLIGAKPDEIYFTSGGTERDNWVXLKGTAWLNANLARILITTSIEPPAVINTMKQLEKLGFEVTYLPVDRRGFIHIDDLKAAIRKDTILVSIMAVNNEIGSMQPIVQAARVLDNYPNIHFHVDAVQAVGKGLDAALQDPRIDFLSFSGHKFHAPRGTGFIYAKEGRMLDPLLTGGGQEHDWRSGTENVPAIAAMAKSLRLLLANEDANVARQQAVRKRIFEHVSQKPKVTMFSQLTPDFAPHVLCFAIAGVRGETIVHAFEDHQIYISTTSACSSKKGTESSTLAAMHTDPKIATSAIRVSLDEANTLDEADAFNAAFDTIYAKFAKLDKATV

SEQ ID NO: 71 - protein, 262 aa, Lactobacillus rhamnosus
MPTKIGLHYNKIGVGKTIYFLHGMGLDGHSMAAFYEPRFTSEERHFARLYPDLPGMGNSPATSALQSADDVLAQVHAFIQATSEGPCYLVGHSYGGYLALGLLARFPDEFSGAFLTAPVVLAEKTARTVATLKHLISAPVTSQSPEFTDYQHMNVVINPSTWRQYQELILPGLKTFNRDFWVAMKNRHAYRLSIESRLTSLIKSPVTLVLGENDNEVGYQDQVVFAHKGAHMTTTVIPNAGHNLMIDAPEAVMTAFHQFLHK

SEQ ID NO: 72 - protein, 309 aa, Lactobacillus rhamnosus
MVTAADNITGLIGNTPLLKLNRVVPEGAADVYVKLEFFNPGGSVKDRIALAMIEDAEYKGVLKPGGTIVEPTSGNTGIGLALVAAAKGYHLIITMPETMSVERRALMRGYGAELILTPGADGMPGAIKKAEALSKENGYFLPMQFQNPANPDVHERTTGQEIIRSFDGGTPDAFVAGVGTGGTLTGVGRALRKINPDVQIYALEAAESPMLKEGHGGKHKIQGISAGFIPDVLDTNLYQDIIEVTSDQAIDMARHVSHEEGFLPGISAGANIFGAIEIAKKLGKGKSVATVAPDNGERYLSTDLFKFDD

SEQ ID NO: 73 - protein, 270 aa, Lactobacillus rhamnosus
MLKKKLWFLLPLVALVTFTLTACTSASSDTSKNSDVTAELINKNELTIGLEGTYAPFSYRKDGKLEGFEVELGKALAKKIGVKAKFVPTQWDSLIAGLGSQKFDLVLNDISETPARKKVYNFTTPYMYSRYALITRSDNTTIKSLADIKGKTFVEGTGTPNAALAKKYGAKITPSGDFTVSLSLVKEKRADGTINASAAWYAFAKNNSTAGLKSQTLKDSVVKPDEVAGMVSKKSPKLQAALSKGIQELRKDGTLKKLSQKYFGTDLTTK

SEQ ID NO: 74 - protein, 474 aa, Lactobacillus rhamnosus
ICKGRSLKPFGHFIDAITVNREHVLTTAAEALIASAGDALNASHATFNVLNNSDLQFGFVENEDGETVQLSNGLYGQLIRSTNRKLRKEAFEALLRAYESLKNTFAQTLSGQVKAHNFNATAHHYKNARAAAMASNHIPESVYTTLIDQVNTHLPLLHRYVALRKKVLAVDQLHMYDIYTPLTGQPPLTYTLEQAKAEALKALAPLGDDYLEHVREIFDNRYIDVVENKGKRSGAYSGGAYDTNPFILLNWHDAVDELYTLVHETGHSVHSWYTRHNQPYVYGDYPIFVAEIASTTNENLLTDYFLTHSDDPKVRAYILNYYLDGFKGTVFRQTQFAEFEHWIHQQDQQGEPLTATSMSQYYADLNARYYGPEVARDPEIAFEWARIPHFYYNYYVYQYATGFAAASTLAAGISSGEPDAAAHYLDYLKSGSSKYAIDTMKTAGVDMTKPDYLEAAFSVFEQRLTELEKILQKG

SEQ ID NO: 75 - protein, 256 aa, Lactobacillus rhamnosus
SYAPTITLEQAKEDIKNATALMGQDYQAQMMQAFSERWIDFPANQGKDSGAYTAGPYGVHPYVEMTWSNTLPAVYTLIHELGHTAQMVRSQEAHNVLDADFNAYLVESPSTFNELLLTHYLEENAKDPRMKRFALSRLLNDTYFHNFVTHLLEAAFQREVYNLIDNGETFDAARLNAITRKVLTDFWGSAVELEPGAELTWMRQSHYYMGLYSYSYSAGLTVATQAFQAIEQQGQPAVDRWLRYLSLGDSLDPVEA

SEQ ID NO: 76 - protein, 641 aa, Lactobacillus rhamnosus
LLGQFGVDLTEQARKGQIDPVIGRDKEISRVIEILNRRTKNNPVLIGEAGVGKTAVVEGLALKIANGDVPAKLQDRHVIRLDVVSLVQGTGIRGQFEQRMQQLIDELKQNKNIILFIDEIHEIVGAGNAEGGMDAGNVLKPALARGELQLVGATTSNEYRQIEKDSALARRLQPVMVEEPSVDETIKILKGLQPRYQDFHHVKYTEGAIEAAATLSNRYIQDRFLPDKAIDLLDEAGSRKNLTIATVDPETIKAKIADAEKQKQAALKQEDYEKAAFYRDQVTKLEDMAKKQSNLPDNEIPTVTEKDMEKIVEEKTNIPVGELKAQEQAQLKNLASDLEQHVIGQNEAVDKVARAIRRNRIGFNKTGRPIGSFLFVGPTGVGKTELAKQLAKELFGSEDAMIRFDMSEYMEKFSVSKLIGSPPGYVGYEEAGQLTEKVRRNPYSLILLDEIEKAHPDVMNMFLQILDDGRLTDSQGRTVSFKDTIIIMTSNAGSTDAEANVGFGATLSGKTHSVLDQLGNYFKPEFLNRFDDIVEFKPLSKDDLLKIVSLMINDTNNNLKSQGLTIHVTDPVKEKLVTLGYNPSMGARPLRRVIQEQIEDRVADFYLDHPNAKELEARISNGEITVGEPAKAEASSKTAKK

SEQ ID NO: 77 - protein, 481 aa, Lactobacillus rhamnosus
TKSVVGVAPESQLLAMKVFTNSDTSATTGSSTLVSAIEDSAKLGADVLNMSLGSVSGNQTLEDPEIAAVQNANESGTAAVISAGNSGTSGSGTEGVNKDYYGLQDNETVGTPGTSRGATTVASAENTDVINQAVTITDGSGLKLGPETVQLSSNDFVDSFDQKKFYVVKDASGKLSTGDAGDYTADAKGKIAIVKRGSLTFTDKQKYAEAAGAAGLIIVNNDGTSTPLTSISLTATFPTFGLSNTTGQKLVDWVTAHPNDSLGVKIALALLPNQNYKADRMSSFTSYGPVSDLSFKPDITAPGGNIWSTQNNNGYTNMSGTSMASPFIAGSQALLKQALNNKDNEFYADYKQLKGTALTDFLKTVEMNTAKPINDINYDNVIVSPRRQGAGLVDVKAAIDALEKNPSTVVSENGYPAVELKDFTSTTKTFKLTFTNRTKHQLTYQMTSNEDTNAVYTSATDLESFIQSSKMAKLIHERGAA

SEQ ID NO: 78 - protein, 331 aa, Lactobacillus rhamnosus
MTINWQQEVEKLEPQLLSDLTTLLKINSERDTDHQTDEYPLGPGPAKALEAFLAIAQRDGFKTLNVDHVAGRIELGDGDEIFGLFGHVDVVPAGPGWQTDPFDPVIRDGKIYGRGTSDDKGPSIAAYYALKLIRDLKLPINKKIHFILGTDEESDWVGIHRYLETEPAPDFGFSPDAEFPIINGEKGIASFEIVQKPIAAATADLTLNHFSAGIRPNMVPQEAKAVLSGPLPEAFVTQAEKWAAEQEVTLTLTLGNPTTIELIGKGAHAQEPKDGKNAATYLATLLADLPFDPAGKAYLTMIANHLHLDSRGHHLGINYTDKLMGDLTASP

SEQ ID NO: 79 - protein, 344 aa, Lactobacillus rhamnosus
  VARIANT (1)...(344): Xaa = Any Amino Acid
GKMSLYAGGPDERLTPLIDGRRHVTDFALTPDHRGVVFTESTMTIPSRLVYFDLASEEEQVLYDPNRQVTRHLGLVTPQTFNFQRDGFEIEGWYFPPQQASSSHPAILYVHGGPAVGYGYTFFHEMQYLAAKGYGVICRNPRGGLGYREAFTGAVIKHXPAGDYEDCLASGEEALKLDTTIDPQRLFVTGGSYGGFMTNWIVTHTHRFKAAVTQRSISNWLSMYGTSDIGYYFTPWELEGKWTGDLSDVQGLWDFSPLAHIDHARTPTLVMHSENDERCPIGPSRKVDHRSQTAWCXNQVHAFPKVKSXFVPAAGLPNLRVARLQAIVDWFDAHQAQPQMAKGE

SEQ ID NO: 80 - protein, 558 aa, Lactobacillus rhamnosus
HLIGATTLDEYRENIEKDKALERRFQRVLVQEPTVEDTISILRGLKERFEIFHKVRIHDSALVAAATLSNRYITDRFLPDKAIDLVDEACATINVEMNSRPTELDVAERKQMQLEIEQQALKNESDPASKKRLENANAELANLKEKTNKLKAQWEAEKKDIRQLNEKKSAIDKAKHELEDAQSRYDLETAARLQHGTIPQLEKELQAMEHSDRPQSWLVQESVTANEIAAVISRETGIPVAKLVEGDRQKLLHLAGNLHQRVIGQDEAVTAVSDAVLRSRAGLQDPSRPLGSFLFLGPTGVGKTELAKALAEDLFDSEKHMVRIDMSEYMEKASVSRLVGAAPGYVGYEQGGQLTEAVRRNPYTIVLLDEIEKANPDVFNILLQVLDDGRLTDGQGRTVDFKNTIIIMTSNLGSEYLLDGVQKDGTVSQQAKDQVRQLIGKAFKPEFLNRIDDIIMFHPLSLDDVKKIAVKDLHELGTRLADQQISLDITPEAQTWLADKGYDPAFGARPLQRLITSAVETPLAKELIRGTIQPGQEVVITVADDQLQFKAKQVVAKA

SEQ ID NO: 81 - protein, 292 aa, Lactobacillus rhamnosus
ISAIIVIVEENNVAARELILAFESSCDETSVAVVENGTKILSNIIATQIKSHQRFGGVVPEVASRHHVEQITLVTDAALKEAGVTYTDLTAVAVTYGPGLVGALLIGVRAAKPIAYAHHLPLIPVNHMAGHIYAARFVKPLVYPLLALAVSGGHTELVYMRAAGEFEIIGDTRDDAAGEAYDKVGRILGIPYPAGKEVDRLAHLGHDTFHFPRAMDKEDNLDFSFSGLKSAVINTVHHADQIGESLSREDLSASSQASVVHVMVLKSQSAIAEYPVIQVVIAGGVADNQGLK

SEQ ID NO: 82 - protein, 244 aa, Lactobacillus rhamnosus
MIFRKPQPFEYEGTDTGVVLLHAYTGSPNDMNFMARALQRSGYGVYVPLFSGHGTVEPLDILTKGNPDIWWAESSAAVAHMTAKYAKVFVFGLSLGGIFAMKALETLPGITAGGVFSSPILPGKHHLVPGFLKYAEYMNRLAGKSDESTQILAYLPGQLAAIDQFATTVAADLNLVKQPTFIGQAGQDELVDGRLAYQLRDALINAARVDFHWYDDAKHVITVNSAHHALEEDVIAFMQQENEG

SEQ ID NO: 83 - protein, 433 aa, Lactobacillus rhamnosus
LGIFFFKRFRKLHLFDPLNYPEETFQSFDSAFNNGADYVELDVHESADGVIVIQHDTTIQRTTGANLAIAKTNFAQLQQYHTKNGEPIHSLEELFAHEQQTKHKFLIETKIVKGEPHPHLEDKVAALIKQYHMENRVMFHSFSAASLKRLQAALPNIPRILIVGSLKRINFDVLTYVDGINLSSDLVTPQLVTQLHDLGKKVYVWDEMNEDRAKWTWLVNLNIDGVVTNYTSLGHEFQTLKAAAVTTSINDLGANSSLAALPVYENPYQPLLRSERLAPQTPIMISSMVSLAGSTYYQIGDNAFVPAETINLAPEAGWASLFLHQRIVITSRHFKVPVHADPLHQQAITGHVGNHKCYRVLAARYQSGQLYLKTKIGWLNAKDLQVLPTAENMRIWLTLYRSIPENQKPLLHWALGDTAFDTPLLNASVLNIG

SEQ ID NO: 84 - protein, 448 aa, Lactobacillus rhamnosus
MELAKLAVDETGRGVWEDKAIKNMFATEEIWHSIKNNKTVGVINEDKQRGLVSIAEPIGVIAGVTPVTNPTSTTIFKSEISIKTRNPIIFAFHPGAQKSSARALEVIREEAEKAGLPKGALQYIPVPSMEATKTLMDHPGIATILATGGPGMVKSAYSSGKPALGVGAGNAPAYIEASANIKQAVNDLVLSKSFDNGMICASEQGAIVDSSIYDAAKKEFEAQGAYFVKPKDMKKFESTVINLEKQSVNPRIVGQSPKQIAEWAGIRIPDDTTILIAELKDVGKKYPLSREKLSPVLAMVKADGHEDAFKKCETMLDIGGLGHTAVIHTADDELALKFADTMQACRILINTPSSVGGIGDLYNEMIPSLTLGCGSYGGNSISHNVGTVDLLNIKTMAKRRNNMQWMKLPPKIYFEKNSVRYLEHMESIKRAFIVADRSMEKAGFRQDH

SEQ ID NO: 85 - protein, 158 aa, Lactobacillus rhamnosus
VLVNNAGITDDMLAMRMKPASFAKVVQVNLDGTFYVTQPAFKKMLKARAGVIINLASVVGLTGNIGQANYAASKAGIIGLTKTLAREGAMRGVRVNAIAPGMIATDMTAALSQSSQDQILAEIPLKRFGQPEEIAHTARFLVENAYITGQTVTVAGGL

SEQ ID NO: 86 - protein, 334 aa, Lactobacillus rhamnosus
MYHAAADRYEKMPVRHAGKTGLMLPVISLGLWQHYGNLDPFGPRRSVILDAFDRGVFHFDVANHYGNGDREPGFGSSERLLGQILATDLKPYRDELVISTKVGYEIHPGPYGVGTSRKAVIQGLNDSLKRLQLDYVDIYYAHRFDDTVALEETVNALDQTVRDGKALYIGISNYDTKQTKEAIAMFKDLHTPFVLNQYSYNMFNRTAETSGLIDALKADGAGLIAYGPLSEGLLSDRYLKGIPDTFKIHPTNKATFAKGKEAVVKQLNALNEIAHDRDQTLSQMALAWLLRDPVVTSVIIGTTSVEHLQDNLKATEHLTFTAEEIQQIDDILNA

SEQ ID NO: 87 - protein, 413 aa, Lactobacillus rhamnosus
AVALPLLGVLAIAATHAEGVYDIGRPLGRFFALAFMVLIHATIGPMFGTPRTATVSFTTGVLPMLPKAWQQGGLLVFSALFFGAAFFLSYKERKITTAVGKVLNPVFLLLLFFVFFIGFLHPMGNPAAQTVTAAYKNGGSFMSGFLQGYNTMDALAALAFGVTVVTAVRGLGLKNDDHVAKATAKAGVMATSWIALIYVALIVLGSMSLAHFKLSAEGGTAFNQVGTFYFGTVGHPAWQPCLTLTCLNTPVGFVRAFPHDFHRHFPKVSYQVWLGLTSFLSFLTANFGLEQIIAWSVPMLMFLYPFSMVLILLSVFGKAFHHDPLVYRIVVAFTIVPAVLDMFAAFPAVVSQSSLGLALHSFQLHFLPFSAMGLGWLVPAGVGLVLGLVAHAVKVRKAVAATHLEAEQTQLVH

SEQ ID NO: 88 - protein, 627 aa, Lactobacillus rhamnosus
MADNHKAQTTKQPSGPRMGPGRGGLVEKPKNFWGTTARLFGYMRNRLIGIIAVLVLAIASTVFQIRTPKILGEATTEIFKGVMKGQAEQKAGIAVGNYPIDFDKIKQIILIVLVLYLGSALFSFLQQFIMTRISQNTVYQLRKDLKHKMKTVPIKYYDTHSNGDIMSRAINDMDNIASTLQQSLTQMVTSAVMFVGTIWMMLTISWKLTLIALVTIPLGLIVVGIVAPKSQRFFAAQQKALGLLNNQVEETYGGQVIIKSFNREDDEVEAFEGQNQAFYDAAWKAQFVSGIIMPLMIFLNNIGYVFVAIMGGIEVSNGTITLGNVQAFLQYMQQFSQPISQLANLANTIQSTIASAERIFAVLDEEDMQDEPSGVPAVANDPNKLVMDHVQFGYTPDALLLKDYNLQVKPGEMVAIVGPTGAGKTTIINLLERFYDISGGSIRLNGTDTRDMKREDVRAHFAMVLQDTWLFTGTIWDNLKYGREDATDDEVLAAAKAAHVDNFVRQLPDGYNTILNEEASNISQGQRQLLTIARAFVADPEILILDEATSSVDTRTEIHIQHAMNRLLTDRTSFVVAHRLSTIRDADKIIVMNHGSIVETGNHDELMAKNGFYADLYNSQFSGNVAI

SEQ ID NO: 89 - protein, 202 aa, Lactobacillus rhamnosus
TTRLSSLITEYLDSQLAERRSMHGVLVDIYGLGVLITGDSGVGKSETALELVQRGHRLIADDRVDVYQQDEQTVVGAAPPILSHLLEIRGLGIIDVMNLFGAGAVREDTTISLIVHLENWTPDKTFDRLGSGEQTQMIFDVPVPKITIPVKVGRNLAIIIEVAAMNFRAKSMGYDATKTFEKNLNHLIEHNEANDQKSSEEK

SEQ ID NO: 90 - protein, 341 aa, Lactobacillus rhamnosus
  VARIANT (1)...(341): Xaa = Any Amino Acid
MSISTRANKLDGVEQAXVAMATEMNKGVLKNLGLLTPELEQAKNGDLMIVINGKSGADNEQLLVEIEELFNTKAQSGSHEARYATIGSAKKHIPESNLAVISVNGLFAAREARQALQNDLNVMLFSDNVSVEDELALKQLAHEKGLLMMGPDCGTAIINGAALCFGNAVRRGNIGIVGASGTGSQELSVRIHEFGGGVSQLIGTGGRDLSEKIGGLMMLDAIGMLENDPQTEIIALISKPPAPAVARKVLERARACRKPVVVCFLDRGETPVDEQGLQFARGTKEAALKAVMLSGVKQENLDLHTLNQPLIADVRARLQPQQKYIRGLSAAARCATKPCSR

SEQ ID NO: 91 - protein, 409 aa, Lactobacillus rhamnosus
QILNNPFLNKGTAFTQEERNQYGLNGLLPPAVQTLDQQVKQAYAQLQTKPTDLAKRQFLMTLFNENHVLFYKLFSEHINEFMPIVYDPTIADTIENYSALFVNPQNATYLSIDDPDHIESALKHSADGRDIRLLVVSDAEGILGIGDWGTQGVDISVGKLMVYTAAAGIDPSQVLPVVLDVGTNNEGLLNDDLYLGNRHKRVYGEKYHHFVDKFVAAAEKLFPNLYLHFEDFGRSNAADILNQYKDKITTFNDDIQGTGIIVLAGLLGAMNISKQKLTDQVYLSFGAGTAGAGIASRVYEAFVEEGLSPEEAKKHFYLVDKQGLLFDDMTDLTPEQKPFARSRSEFANADELTTLEAVVKAVHPTVLVGTSTVPGTFTESIVKEMAAHTDRPIIFPLSNPTKLAEAKAD

SEQ ID NO: 92 - protein, 386 aa, Lactobacillus rhamnosus
  VARIANT (1)...(386): Xaa = Any Amino Acid
MIKPEKTINGTKWIETIQINAEERATLEDQYGVDEDIIEYVTDNDESTNYVYDINEDDQLFIFLAPYALDKDALRYITQPFGMLLHKGVLFTFNQSHIPEVNTALYSALDNPEVKSVDAFILETLFTVVDSFIPISRGITKKRNYLDKMLNRKTKNSDLVSLSYLQQTLTFLSSAVQTNLSELDLNGSDALQQIIELLNQHPLDXAPDEKGAYSNSNYYLLGHIITQVANMPLSDFLNQHFFEPLAMTKTQLGTQHADANSYDDLDFTNGKPVALGRGHYQGGDGAVVSSLADLAIWARAVLQRRILPESAWDEALTLTHDFYGMGWMKSRTQHWLSHNGHIFGYWAFFDVSFEKQLAQITLTNMSPGVETLKKWQEEMANWRASL

SEQ ID NO: 93 - protein, 357 aa, Lactobacillus rhamnosus
LDNQDADFKPTIQILDEVGKVVNPDIMPDLSDDQLVDLMSKMVWQRVLDQRATALNRQGRLGFYAPSAGEEASMIGSHAAMKSSDWLLPAYRDLPQLIQHGLPLDKAFLWSRGHVAGNEYPEDFHALPPQIIIGAQYVQTAGVALGLKKNGSDEVAFTYTGDGGTSQGDFYEGVNFAGHFKAPALFIVQDNGFAISVPRASQTAAKTLAQKAVAAGVPGVQVDGMDALAVYEVTKEARAWAAAGNGPVLIETLTYRYGPHTLSGDDPTRYRSKETDELWQKRDPLIRMRNYLTDKGLWSKDKEDALIEKVKDEIKDAINKADKAPQQTVSRFLKDTYEVAPQNVAEQLAEFQGKESK

SEQ ID NO: 94 - protein, 436 aa, Lactobacillus rhamnosus
SVLNINGGNLTLTDDGVSAGTLTGGGFLNISGGVLDITGGNHTFAVSTIIAKDATVRMNDVSGLGTGNISNAGTLSLTHASGLLSNNLSGSGTVSLINSDTQISGNNSNYSGLFVVDTSSQLTATGAQNLGIASVSNRGILQLNNTTDWQLINNVTGTGNVRKTGSGSLTVRSNAAWSGQTDIDDGSLILGQSDAPVMLASSLVNIAKNGKLTGFGGVVGNVTNSGSLDLRSAAPGNILTIGGNYTGNNGTLLINTVLDDSSSATDKLVIKGDASGKTRVAVTNVGGSGANTLNSIEVIHVDGNAANAEFIQAGRIAAGAYDYTLGRGPGSNYGNWYLSSSKNTPEPRPDPEPTPEGHDNNLRPEASSYTANIAAANTMFVTRLHERLGQTQYVDAITGEPKATSMWMRHEGGHNRWRDGSGQLKTQSNRYVIQLG

SEQ ID NO: 95 - protein, 215 aa, Lactobacillus rhamnosus
MKILITGAQGQLGTELRHLLDARGITYRATDAKDLDITDEAAVNQYFADYQPDVVYHCAAYTAVDKAEDEAKALNQLVNVDGTRNLAKAAAKVDATLVYISTDYVFDGDSKEIYTVDDQPAPRNEYGRAKYEGEQQVQKYLKKYYIIRTSWVFGEYGHNFVYTMLNLAKTHKELTVVDDHQESFSVSSSRTFVKYQHEHLIYSRPVPYRPHLPGI

SEQ ID NO: 96 - protein, 640 aa, Lactobacillus rhamnosus
  VARIANT (1)...(640): Xaa = Any Amino Acid
MLGGKQMPEVKKFEAGTYDVIVVGAGHAGXVKPALAAARMGEKTLLLTISLEMLAFMPCNPSLGGPAKGIVVREIDALGGEMGKNIDRTYIQMRMLNTGKGPAVRALRAQADKAAYHRSMKHVIEDTPHLDLRQGLATEVLVEDGKAVGIVAATGAIYRAKSIVLTAGTSSRGKIIIGELMYSSGPNNSLPSIKLSENLEQLGFKLRRFKTGTPPRVNGNTIDFSKTEEQPGDKTPNHFSFTTPDSVYLKDQLSCWMTYTNATTHQIIRENLDRAPMFSGVIKGVGPRYCPSIEDKIVRFADKPRHQLFLEPEGRDTSEYYVGDFSTSMPEEIQLKMLHSVAGLEHAELMRAGYAIEYDVIEPWQLKATLETKVVENLYTAGQMNGTSGYEEAAGQGIVAGINAARRAQGKGPFTLKRSDAYIGVMIDDLVTKGTNEPYRLLTSRAEYRLLLRHDNADLRLTPMGHELGLISDQRYAVFLAKRQAITDELARLEHTRLKPKDVNPWLEAHHFASLKDGVLASDFLKRPEINYQTLEQFLPENPTLDHRVIEQVEIQIKYAGYIAKEEXQCAKLKRLEGKKIPARINYEAINGLATEARQKLVKIQPETIAQASRISGVNPADVAILSVYIEQGRISKVAQ

SEQ ID NO: 97 - protein, 254 aa, Lactobacillus rhamnosus
PLSTMMLAGIRDILVISTPRDIDRFQDLLKDGKQLGLNISYKIQEKPNGLAEAFIVGADFIGDDSVCLILGDNIFYGSGLSKLVQRSAAKTTGATVFGYQVNDPERFGVVAFDEQHHVQSIVEKPEHPESNFAVTGMYFYDNQVVDIAKNLKPSPRGELEITDVNKAYLERGQLDVELLGRGFAWLDTGTHESLHEAASFIETVQKRQNLKIACLEEVAYRMGYIDRDQLRKLAQPLKKNDYGQYILRLADEED

SEQ ID NO: 98 - protein, 312 aa, Lactobacillus rhamnosus
MAINLVGINDANLTLIEEGLNVRISPFGDELRISGETEAVSLTLQLLEAATKLLAQGIKLSPQDIASAVAMAKRGTLEYFADMYSETLLRDAKGQPIRIKNFGQRQYVDAIKHNDITFGIGPAGTGKTFLAVVMAVAAMKAGQVERIILTRPAVEAGESLGFLPGDLKEKVDPYLRPVYDALYAVLGKEHTDRLMDRGVIEIAPLAYMRGRTLDNAFAILDEAQNTTQAQMKMFLTRLGFGSKMIVNGDVTQIDLPHNAKSGLLQAEQLLKGISHIAFTQFSAQDVVRHPVVAKIIEAYGKHDLQLQKQTKE

SEQ ID NO: 99 - protein, 280 aa, Lactobacillus rhamnosus
MKKFDKMMDWLADVYVNALNVIHYMHDKYYYEAAQLALKDTRLNRTFATGISGLSHAVDSISAIKYGHVKAIRDENGVAIDFVADNDDYPRYGNNDDRADNIAKWLVKTFYNKMNTHHLYRGAKLSTSVLTITSNVVYGKNTGTTPNGRQKGEPFSPGANPAYGAEKNGALASLMSTAKIPYHYATDGISNTFGVTPNTLGHDDETRKDTLVHMVDGYMENSGMHLNINVFNKETLIDAQKHPEEYPTLTVRVSGYCVYFADLTKEQQDDVIARTFFDEM

SEQ ID NO: 100 - protein, 447 aa, Lactobacillus rhamnosus
MAFSKETRTQTIDQLKQTELDLLIVGGGITGAGVAIQAAASGLKTGLIEMQDFAEGTSSRSTKLVHGGIRYLKTFDVGVVADTVKERAVVQGIAPHIPRPFPMLLPIYQEAGSTFDMFSIKIAMDLYDRLANVEGSQYANYTVTKDEILQREPHLASDGLQGGGVYLDFVNNDARLVIENIKEAAELGGLMASRVQAIGVLHDDAGQVNGLQVKDLLDGSVFDIHAKLVINTTGPWSDKFKALDQAEDQTPTLRPTKGVHLVVDGSRLPVPQPTYMDTGLNDGRMFFVVPREGKTYFGTTDTDYHGDFNHPQVEQADVDYLLKVINKRYPQSHITLDDIEASWAGLRPLIANNGSSDYNGGGANTGKVSDDSFEALIRVVDDYEDNQATRADVEHAISKLETAHAEAALSPSQVSRGSSLRQADDGMITLSGGKITDYRKMAAGALA

SEQ ID NO: 101 - protein, 242 aa, Lactobacillus rhamnosus
DLFCPDITADILTRKDDLGSDKPIVDVILDRAGNKGTGKWSSQSALELGVPQSVITESVYARYISAMKQERVAASKVLPKPVGNVTIDKKEAIEMIRKALYFSKLMSYAQGFEQMRVASDNYDWNLQYGELAKIWRAGCIIRARFLQNITDAYDKKPDLQNLLLDDYFLNIAKNYQESVRDLVGLAVKAGVPVPGFSAAISYYDSYRAPVLPANLTQAQRDYFGAHTYERTDRDGIFHYTWY

SEQ ID NO: 102 - protein, 323 aa, Lactobacillus rhamnosus
EDFFIQISATQHHIPDCCDQIPTGDFSFFDNTLDVANLLNIVPKRYQDLNLSPLDTYFAQARGYQGEAGDVKALAMKKWFNTNYHYLVPEFDRDTKIQVTDWQLFVQFEEAKALGINGRPTLIGPYTLLKLSRFIDVVPDDFVADLISAYTTIIDRLHDAGADWVQLDEPALVYDQTDADLALFERLYTPILTQKKAAKILVQTYFGDLTDSFDRIQKLPFDGFGLDFVEGYANLDLLKQHGFPAHATLFAGIVNGKNIWRTHYADALATIKQLATITDKLVLSTSTSLLHVPYTLRNETHLKPEEKQYLAFAEEKLNELHEL

SEQ ID NO: 103 - protein, 296 aa, Lactobacillus rhamnosus
GPAIFGFIPMQDGSPAPGLSNITAEGWFPHGGLPILMTMVAVNFAFSGTELIGIAAGETENPRKVIPVAIRTTIARLIIFFIGTVFVLAALIPMQQVGVEKSPFVLVFEKVGIPYAADIFNFVILTAILCAANSGLYASGRMLWSLSNERTLPACFARVTKNGVPLTALSVSMLGGVLALFSSVVAPNTVFVALSAISGFAVVAVWLSICASHFVFRRRHLQQGKALSELHYRAPWYPLVPVLGFVLCLVACVGLAFDPAQRIALWCGLPFVALCYGAYFLTQPRNAKQEPEHVAE

SEQ ID NO: 104 - protein, 474 aa, Lactobacillus rhamnosus
MRKQLPKDFVIGGATAAYQVEGATKEDGKGRVLWDDFLEKQGRFSPDPAADFYHRYDEDLALAEAYGHQVIRLSIAWSRIFSDGAGAVESRGVAFYHRLFAACAKHHLIPFVTLHHFDTPERLHEIGDWLSQEMLEDFVEYARFCFEEFPEIKHWITINEPTSMAVQQYTSGTFPPAETGHFDKTFQAEHNQIVAHARIVNLYKSMGLDGEIGIVHALQTPYPYSDSSEDQHAADLQDALENRLYLDGTLAGDYAPKTLALIKEILAANQQPMFKYTDEEMAAIKKAAHQLDFVGVNNYFSKWLRAYHGKSETIHNGDGSKGSSVARLHGIGEEKKPAGIETTDWDWSIYPRGMYDMLMRIHQDYPLVPAIYVTENGIGLKESLPAEVTPNTVIADPKRIDYLKKYLSAIADAIQAGANVKGYFVWSLQDQFSWTNGYSKRYGLFFVDFPTQKRYVKQSAEWLKQVSQTHVIPE

SEQ ID NO: 105 - protein, 258 aa, Lactobacillus rhamnosus
MTTLKSFRVINKVDLPSAQPDVVKEEIEEMIGLDASDAILASGKTGLGVPEILERIVSDIPAPSGDVNAPLQALIFDSVYDDYRGVVLDVRVKEGQVKVGDTIQLMSNGKQFQVTEVGVMSPKAVKRDFLMVGDVGYITASIKTIQDTRVGDTVTLADRPAAAPLKGYRKITPMVYSGLFPVDNAKFNDLREALEKLQLNDAALEFEPETSQALGFGFRCGFLGLLHMDVVQERLERDYGLDLIMTAPSVDYQVALTD

SEQ ID NO: 106 - protein, 418 aa, Lactobacillus rhamnosus
MDVTTIDLEQMGRAAKAAATVLSQLTTAQKNAGLLAMVTALETHTETILGANHEDLKAAASLPAKFTDRLVLTAERIADMAAGVRQVAALPDPTAQTDKAWVNHAGLNIAQKRVPLGVVGMIYEARPNVTVDAAALTFKSGNAVILRGGKEALHSNLALATVLQAALTAQGLPKDAIQLITDPKREVANQMMHLNGYIDVLIPRGGRGLIKAVVEQATVPVIETGAGNCHIYVDAYAQAQMAIDIVVNAKVQRPSVCNAAEKLLIHADVANAQLPLIAAALQAHGVELRGDERARAIVPNMQIATEEDWDTEYNDLIMAVKVVDSEEEAIAHINAHNTKHSEAIITDNYQNSQQFLQQVDAAVVYVNASTRFTDGFEFGFGAEIGISTQKLHARGPMGLAALTTIKYQVLGNGQVREG

SEQ ID NO: 107 - protein, 163 aa, Lactobacillus rhamnosus
MTAFLWAQDRDGLIGKDGHLPWHLPDDLHYFRAQTVGKIMVVGRRTYESFPKRPLPERTNVVLTHQEDYQAPGAVVVHDVAAVFAYAKQHPDQELVIAGGAQVFTAFKDDVDTLLVTRLAGSFEGDTKMIPLNWDDFTKVSSRTVEDTNPALTHTYEVWQKKA

SEQ ID NO: 108 - protein, 95 aa, Lactobacillus rhamnosus
QGCNLYGIATALARISKAILNDENAVLPLSVYMDGQYGLNDIYIGTPAVINRNGIQNILEIPLTDHEEESMQKSASQLKKVLTDAFAKNDIETRQ

SEQ ID NO: 109 - protein, 323 aa, Lactobacillus rhamnosus
MLTKRQLLVLKEIIRLFTESGQPVGSKTLMQELPVHVSSATIRNDMASLEDAGLITKTHSSSGRVPSTQGYRYYLDHLVEPVRVSHRELATIKQAFGQRYNKMDEIVAQSAQILSNLTSYTAISLGPEVNNIKLTGFRLVPLGNHQVMAILVTNNGNVENQVFTVPESISSDELEKAIRIVNDQLVGLPLIQVAQRLKTDVPSMLMQYLTSPEGFLDIFGNVLKSAASERFYVGGRLNLMDYLGDSDIHELKKIMSLIDADHGDLTELLGGPVRQTPVTVRLGPELKPIDLANLKLITASYDVGDHGTGMIALLGPTQMPFSK

SEQ ID NO: 110 - protein, 306 aa, Lactobacillus rhamnosus
MFEHGFIEVHDANQNNLQHVNVKIPKDAITVFVGRSGSGKSSLVFDTIAAESRRELNETFPSFTQQYLPKYGQPDVGSIDHLPVAIVVEQKRIGKNARSTLATYTGIYSLLRLLFSRAGKPFIGYSDTFSFNLPQGMCPTCQGLGYVDDIDVSKLIDPNKSLNQEAITFVSFGPDTWRWRRYAYSGLFDNDKPLRDYTPEEMKLLLYAPQQTLKHAPAKWPRTALYEGVVPRIKRSIIGKKEAEHHKAALAEIVTRKPCPDCQGTRLRPEVLTCLINQTNIAQVLQMDLVNVRHFLKNIQVPLVQD

SEQ ID NO: 111 - protein, 253 aa, Lactobacillus rhamnosus
MTQSADPHAPPLLAKWRQWDTDRHKSSPFEHPNPEVPGASDRLLTEEIAGIFILGTNGESYVLAEDEKLAFVEHVIDYVHGRTKVLVGTGLNGTAETIRFSQKVASLKPDAITLVAPSFVAPSQQELVDHVAAIIHADDIPVLLYNMPAKTGINIEPASLKQLSKYENLIGIKDSSGKWENFDGYLANRPERPFSVIMGSDGRILESFQHGGNAAIASTANLLTANNVALYQAFVNDNIEKAQKFQDRIQPLR

SEQ ID NO: 112 - protein, 217 aa, Lactobacillus rhamnosus
MAMSPINAPTIKGTSKATDQGVDWSRYQGDNGVFGYSTDKFGISQIGGYSGYGTYEQTTYKTQVASLIAAGKRAHTYIWWQNIDNTNFAKQVLDHFLPEIQTPKGSIVALDYEAGSTNTATLLWALDYIRDAGYTPMLYGYKSFLMSHIDLSQIASRYQLWLAEYPDYNVTTVPNYGYFPSFDNVGIFQFTSTYRAGGLDGNVDRSPRTLTKPRCCQ

SEQ ID NO: 113 - protein, 282 aa, Lactobacillus rhamnosus
MATSHFKASKQLCYYLLGVLGIAVVFGLGLFGGYFVSIIDATPIPTETAMKATLSNTSRTSSMYFAHNVKLSDVKSDLYSTKVNLNEMSPWLTKAIIATEDEDFYRHNGIVPKAVIRAFFSDLTGMGSQTGGSTLTQQVVKMMFLNSETTFKRKAAEIMLARRLNNHFSKNTILATYLNVATLGRNNKGQNIAGVEAAAQGLFGVSAKEVNLPEAAFIAGLPQSPFVYTPYTADGKLKTSLKAGINRQQTVLFRMYRAGVISHRQYVAAKSFDPLVSTCRRA

SEQ ID NO: 114 - protein, 418 aa, Lactobacillus rhamnosus
MTTVGHIRNELLATFRKNPNIDYSVQTLSRALKLSEGGDFKVLVQALNGMENDNLIHANHEGRYALGGAPKVLTGTFRGNEKGFGFVAVEGLDNDVYVPAMNTDFALDGDTVEVRIVREARPNDSRGPEGEITKIVQRSLTTLVGEFKPFSDKDRAKSGFIGMVVSHEKKLKNFPVYVKDTGNIPQLGDMTVTEITEFPTEYHPKLMYGIVVETLGNKNDPGVDIMSLVMQNHIKTEFPDEVMDQTNAIPDHVTPEERVGRKDITDQAVVTIDGDDSKDFDDAVVVWKLPNGNFHLGVHIADVSHYVTEGSALDQEAFDRGTSTYLVDRVIPMLPFRLSNGICSLNPGVDRLAMSCDMEIDHDGHVVNHEIYQSVIKSHARMTYNNVNKIVTDPDPEVMAEYQELVPMFEDMVELHQI

SEQ ID NO: 115 - protein, 295 aa, Lactobacillus rhamnosus
YENEIIITIRAGRKNHPLLLSANPQYARVQITHIPFTNPDVPATFTMTLRKYFNAATLTEIHQVQNDRVLHFEFSTRDELGDELGLRLIIEMMGRHSNIFLVSKRTGKIIDLIRHVSADQNRYRPLMPGAPYVEPPKQDKVDPFHDSERIYHELERQVTPSLSRAALLQQHYQGLAKDSAAELALRLNQGDAGWDSFFAALATPEPTITTQGKKAVFTAIPYQSLTGEQQHFPTLSAMLDAYYAQKAEHDRVLQQGGNLIHVIKNVIDKDRKKQRKLKRTLEETEKADDYRIRFK

SEQ ID NO: 116 - protein, 231 aa, Lactobacillus rhamnosus
MWHLPITGYTFYRSDMMPVKKTNAVNLSLFILLLTLEISFSHAVSLNVALIGLASGFLIWRRAFKSLVVLALLPLIPAASTYWAITLHGTDTTYALLLWVRTYAFTALGLVFLIGVDLETLLLWLEQHKLSPNFVYGLLVVIHALPQIMHEVAAIREASLLRGQKLHAWSPMIYVKVIFVAMSWQDQYVKAMYAHGYTEGAARTVHQTIRSSWRGLIAMVGGFVLLNLIDR

SEQ ID NO: 117 - protein, 225 aa, Lactobacillus rhamnosus
MMAKILIVEDHRISRHLLKDVLTPTYTVTQAYDGIQALTAFHREQPDLIILDLMLPNVTGESVLTTIRKTSQVPVLVLTAIQEKAKTVALLQQGANDYLTKPFDIDELLARIQVQLRQVSGQPITTNDQLKVGEIQLDPKRHVVTVNQQTLTLPKKEYDMLALMMRDPHQVFDKSQLYEHVWGEPFLNADNTLNVHISNLRTKINELAHDPKYIISIWGIGVRLI

SEQ ID NO: 118 - protein, 259 aa, Lactobacillus rhamnosus
RFASVPQDPDNLAGIDSNRIAKYQEAFAKAYKRLMEAISSMSISWTIIGAASPRWAQKVFPDAATPEEATELLWEAIFKTTRIDQPDPEAAWKAHDQKLREKAAWLNNEQFDQLHYMAPGTDLVVGLPKNHIWEGAGAFNPRGEEFMANMPTEEVFTAPDFRRIDGTVASTKPLSYGGNILEDMHFTFKDGQIVEAHAKQGDDVLQNLLKTPGARSLGEVSLVPDPSSISQSGLIFFNTLVDENASDHMALGQAYPFSV

SEQ ID NO: 119 - protein, 438 aa, Lactobacillus rhamnosus
VKRALLSVSDKTGLVPFAKGLVERGFELISTGGTHRALAEAGVAVTGVEAVTGFPEMLDGRVKTLHPKIHAGILARRDDPAHMQALADHDIQPIDVVCVNLYPFAATIQRAGVTRAEAIEQIDIGGPSALRAAAKNSDSVWAVVDPADYADVLAGLDQNDADLRQRLAAKVFSATAAYDAQIAHYLDPEPFPEQFTPTYHKRQDLRYGENSHQQAAFYVEPNPDPTSLAAAKQLHGKELSYNNIKDADAALAMLREFKQPAAVAVKHMNPCGIGLGDTLEAAWDKAYAADPMSIFGGIIALNRRVDLATAEKMHKLFLEIIMAPAFDDDAYEILAKKKNVRLLTINTADTPEELGTETTSIYGGLLIQTRDDKAETPADMTVVTEVKPTEAQLKALAFAQTVVKHVKSNAIVVAQADQTLGIGAGQMNRIGSVELALT

SEQ ID NO: 120 - protein, 279 aa, Lactobacillus rhamnosus
MVKRNPNGTRFITLPNGYHLWTQTLAAADSLLTLHGGPGGTNEVFENFATELASFGVRVSRYDQLGSFFSDQPDFSDPANQKRFLNIAYYVDEVENVRQQLGLDHFYLLGQSWGGVLAIEYGLKYSQHLKGLILSSMIDNLDEYLVNINKIRETMFSSDDVAYMQRIEAQHAFTDAKYQQLVRELGEQYLHHAKDPQPRHLISTLATPVYHHFQGDNEFVMVGALRDWDRRADIHRLTMPTYLTFGGHETMPLSAAKRMARTIPNATLHVTPNAGHGQM

SEQ ID NO: 121 - protein, 317 aa, Lactobacillus rhamnosus
MSRRYRQFDANRAGSRSRGGLNLISLGIYEKALPRTESWVERLKMVRDLGFNFLELSVDESDERLARLDWTAAKRAKVRDACWQTGVRIHTLMLSGHRRFPLGSADPAIREKSLTMLCKAIDLASDLGVRNVQLAGYDVYYEPKTLASREYFIENLKRGVAYAAAKEVMLAIETMDDPFLNSLSKIKTIKDEIPSPWLQAYPDLGNLSAWPENNVGRELELGIANIVSVHLKDTQAVTVKSKGQFRDVPFGAGVVDFSGCLRTLKRLDYSGAFTIEMWTEKAADPIQEVKQAKDFFDPLFVQAGFVQEPVAKTNVPS

SEQ ID NO: 122 - protein, 310 aa, Lactobacillus rhamnosus
MTDPIAFLQKLIQIDSANGNELAVARVLQAELEAADIPTKLIPYKDDRVNLVAQLNHGDRVLGFTGHEDVVSPGDENAWTYPPFSGKIVNNTMYGRGTDDMKSGLAAMTLALIHLKQSGFAHPLRFMATVGEEFGAMGARQLTEQGYADDLTGLVVGEPTNKLLKYAHGGTVNYEIDSEGVSVHSSRPEKGVNAIEGLVAFSTPEPHAFDQAPDDPDLGPFRHSITVIKGGDQVNTIPAHAYLRGNLRPTPAANIELVVGLLEKLVDQANKATAANLTLNVLHRFLPVHSDKNGHLVTTANEAIAAVTGK

SEQ ID NO: 123 - protein, 246 aa, Lactobacillus rhamnosus
MKIDIDKTSMIPVYEQIANSLRDMMYGGSLQDGDRLDSEQKMCRNLNVSRGTVRKAIDILLKEGMVKKIHGKGTFVSNPNVEYSLNDQLMSFAESLDNQHLSYTTQVIQQELRPATAKIADMLKIPIDSQYLYLERLRSVADDKLMLIENRINITLCPGIEKVNFNNISLFNEIEELAKRKISFARSTYEALTIGTERGKLLELPSSTPALKMQQTVYLSEKEPVEYGSVWLKGNKYFLTTTLQRR

SEQ ID NO: 124 - protein, 520 aa, Lactobacillus rhamnosus
LVDPFLFTPQLTIEEVKKAGWAYPVFGYLDHEDPFAKLASHIKTVNPNPTKWAIEKDNLAVFKFEAIMKQFPDATFPIDASRFIEKQRLIKTASEIKQMEAAGAQADRAFQAGFNAIKAGATEQEVAAEIDYAMMKEGVMHMSFGTIVQAGVDAANPHGEPMGTKLAPNELVLFDLGTDNHGYMSDATRTVAFGQVTGKPREIFDICLEANLTAMDAVKPGLKASELDKIARDIITKAGYGEYFNHRLGHGIGMSTHEFPSIMEGNDMIVGEDFGMRVSVLASSSSGNATYIETPGHKVLVDAGLSGKKIEALMKSIGRDLTDVDSVFITHEHSDHVRGVGVLARRYPQLNVYANAKTFAALPKSVGKIPEAQLRLFDMGTTLTLGDLDVESFGVSHDAAAPQFYQFHHDGKAFTILTDTGYVSDRVAGTIRDADAYVMECNHDLEMLRTGPYPWPLKQRILSDQGHLSNEDGADALMDVIGLRTKRIYLGHLSPHNNNKATGAFNRGVVVGTTRSGGGS